(19) United States
(12) Patent Application Publication
(10) Pub. No.: US 2025/0029260 A1
(43) Pub. Date: Jan. 23, 2025

(54) NATURAL AND ARTIFICIAL INTELLIGENCE FOR ROBUST AUTOMATIC ANATOMY SEGMENTATION

(71) Applicant: The Trustees of the University of Pennsylvania, Philadelphia, PA (US)

(72) Inventors: Jayaram K. Udupa, Philadelphia, PA (US); You Hao, Philadelphia, PA (US); Chao Jin, Philadelphia, PA (US); Yubing Tong, Philadelphia, PA (US); Dewey Odhner, Horsham, PA (US); Drew A. Torigian, Philadelphia, PA (US); Tiange Liu, Philadelphia, PA (US)

(21) Appl. No.: 18/771,929

(22) Filed: Jul. 12, 2024

Related U.S. Application Data
(60) Provisional application No. 63/513,726, filed on Jul. 14, 2023.

(51) Int. Cl. G06T 7/12; G06T 7/13; G06V 10/25; G16H 30/40

(52) U.S. Cl. CPC G06T 7/12; G06T 7/13; G06V 10/25; G16H 30/40; G06T 2207/20084; G06V 2201/07

(57) ABSTRACT

Methods and systems are described for recognizing and delineating an object of interest in imaging data. Artificial intelligence and natural intelligence may be combined through a plurality of models configured to locate a body region, trim imaging data, perform fuzzy object recognition, detect boundary areas, modify the fuzzy object models using the boundary areas, and delineate the objects.

### CROSS-REFERENCE TO RELATED APPLICATIONS

This application is related to and claims the benefit of U.S. Provisional Patent Application No. 63/513,726 filed Jul. 14, 2023, which is hereby incorporated by reference for any and all purposes.

### GOVERNMENT SUPPORT

This invention was made with government support under R42CA199735 and R01CA255748 awarded by the National Institutes of Health. The government has certain rights in the invention.

### BACKGROUND

Automatic segmentation of 3D objects in computed tomography (CT) is challenging. Current methods, based mainly on artificial intelligence (AI) and end-to-end deep learning (DL) networks, are weak in garnering high-level anatomic information, which leads to compromised efficiency and robustness. Thus, there is a need for more sophisticated techniques for automatic recognition and segmentation in CT.

### SUMMARY

Methods and systems are described for recognizing and delineating an object of interest in imaging data. Artificial intelligence and natural intelligence may be combined through a plurality of models configured to locate a body region, trim imaging data, perform fuzzy object recognition, detect boundary areas, modify the fuzzy object models using the boundary areas, and delineate the objects.

An example method may comprise any combination of the following: receiving imaging data indicative of an object of interest; determining a portion of the imaging data comprising a target body region of the object; determining, based on automatic anatomic recognition and the portion of the imaging data, data indicating one or more objects in the target body region; determining, based on the data indicating the one or more objects and for each of the one or more objects, data indicating a bounding area of an object of one or more objects; modifying, based on data indicating the bounding areas, the data indicating one or more objects in the target body region; determining, based on the modified data indicating one or more objects in the target body region, data indicating a delineation of each of the one or more objects; and causing output of the data indicating the delineation of each of the one or more objects.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to limitations that solve any or all disadvantages noted in any part of this disclosure.

Additional advantages will be set forth in part in the description which follows or may be learned by practice. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate embodiments and together with the description, serve to explain the principles of the methods and systems.

The file of this patent or application contains at least one drawing/photograph executed in color. Copies of this patent or patent application publication with color drawing(s)/photograph(s) will be provided by the Office upon request and payment of the necessary fee.

FIG. 1 shows a schematic representation of the different stages (modules) in the proposed hybrid intelligence (HI) system. AAR-R, automatic anatomy recognition object recognition; BRR, body region recognition; DL-D, deep-learning-based object delineation; DL-R, deep-learning-based object recognition; MM, model morphing

FIG. 2 shows an overall architecture of the object sparsity-specific deep-learning (DL) recognition network employed by the deep-learning-based object recognition (DL-R) module

FIG. 3 shows an illustration of the deep-learning-based object recognition (DL-R) recognition process and the improvement achieved over automatic anatomy recognition object recognition (AAR-R).

FIG. 4 shows an illustration of the smoothing process to obtain the modified fuzzy model FM_{O,M}(I_{B}) from FM_{O}(I_{B}). The exemplar object is thoracic spinal canal. Panel (b) shows the process of obtaining the smoothed centers (red) from the centers computed from bb_{O}(I_{B}). Panels (a) and (c) show surface renditions of bb_{O}(I_{B}) before and after smoothing

FIG. 5 shows neck cases. Examples ranging from near-normal studies to cases with a severe degree of one or more of the deviations listed in Table 12, with representative organs at risk (OARs). Row 4 demonstrates: Left: Axial contrast-enhanced computed tomography (CT) simulation image through the neck shows asymmetric thickening and sclerosis of the maxilla with associated distortion of the extended oral cavity. Middle: Axial non-contrast-enhanced CT simulation image through the neck demonstrates asymmetric fat stranding and loss of intervening fat planes in the right submandibular region and right posterior triangle as well as thickening of the right portion of the platysma muscle related to post-radiation changes with associated obscuration of the right submandibular gland. Right: Sagittal contrast-enhanced CT simulation image through the neck reveals a large mass in the tongue/floor of the mouth in the region of the extended oral cavity.

FIG. 6 shows thoracic cases. Examples ranging from near-normal studies to cases with a severe degree of one or more of the deviations listed in Table 8, with representative organs at risk (OARs). Row 4 demonstrates: Left: Sagittal contrast-enhanced computed tomography (CT) simulation image through the thorax shows degenerative change and exaggerated kyphosis of the thoracic spine with associated distortion of the thoracic spinal canal and other intrathoracic structures. Middle: Axial non-contrast-enhanced CT simulation image through the thorax demonstrates postsurgical changes of prior partial rib cage resection and mesh repair in the anterior left chest wall with associated distortion of the shape of the left chest wall and left hemithorax. Right: Axial non-contrast-enhanced CT simulation image through the thorax reveals decreased volume of the right lung and mild rightward mediastinal shift related to prior partial right lung resection.

FIG. 7 shows an illustration of cases of failure. The expected location of the organ at risk (OAR) is indicated by a region of interest (ROI). Panel (a) shows left submandibular gland (LSmG): Large postsurgical muscle flap containing fat in site of partial left mandibular resection which exerts mass effect upon other structures. Panel (b) shows right submandibular gland (RSmG): Low contrast and surgical clips/calcifications. Panel (c) shows left buccal mucosa (LBMc): Severe streak artifacts from dental implants in this region. Panel (d) shows right buccal mucosa (RBMc): Region of severe streak artifacts from dental implants. Panel (e) shows left lung (LLg): Lack of left lung aeration due to post-obstructive atelectasis caused by left perihilar tumor and left pleural effusion.

FIG. 8 shows 3D surface renditions in different combinations of neck (rows 1, 2) and thorax organs at risk (OARs) (rows 3, 4) from ground truth contours (rows 1, 3) and auto-contours (rows 2, 4)

FIG. 9 is a block diagram illustrating an example computing device.

FIG. 10 shows the complete structure of the neck network.

FIG. 11 shows the structure of the SA module.

FIG. 12 shows an illustration of the morphing process.

FIG. 13 shows Examples of thoraco-abdominal organ segmentation in dynamic MRI using the HI approach. A slice from a dMRI study (top row) and the segmented organ overlaid on the slice (bottom row). From left to right: Skin, Right Lung, Left Lung, Right Kidney, Left Kidney, Liver, and Spleen.

FIG. 14 shows some examples of auto-segmentations of the hemi-diaphragms in dMRI in 3 different studies.

FIG. 15 illustrates the process of transferring the segmented mask to the PET image for a normal Right Lung as an example organ and the phantom used for evaluating TLB.

FIG. 16 shows examples of auto-segmentation in the pelvis using the HI approach on CT images of FDG-PET/CT acquisitions. A slice from a study (top row) and the segmented organ overlaid on the slice (bottom row). From left to right: Skin, Bladder, Rectum, Prostate.

FIG. 17 shows an example of auto-segmentation of a diagnostic chest CT scan using the HI approach showing the segmentation of epicardial adipose tissue in panel (A), heart including epicardial adipose tissue in panel (B), visceral adipose tissue in panel (C), and thoracic muscle tissue in panel (D).

FIG. 18 shows a schematic representation of the proposed HI-system. BRR: Body Region Recognition. AAR-R: AAR object recognition. DL-R: Deep-learning-based object recognition. MM: Model morphing. DL-D: Deep-learning-based object delineation. The dotted modules which were part of the previous system are excluded in the proposed system.

FIG. 19 shows the simple object hierarchy of the thorax body region.

### DETAILED DESCRIPTION OF ILLUSTRATIVE EMBODIMENTS

Image segmentation is the process of delineating the region occupied by the objects of interest in a given image. This operation is a fundamentally required first step in numerous applications of medical imagery. In the medical imaging field, this activity has rich literature that spans over 4 decades. In spite of numerous advances, including via deep learning (DL) networks (DLNs) in recent years, the problem has defied a robust, fail-safe, and satisfactory solution especially for objects that are manifest with low contrast, spatially sparse, have variable shape among individuals, or are sites of frequent imaging artifacts in the body. Although image processing techniques, notably DLNs, are uncanny in their ability to harness low-level intensity pattern information on objects, they fall short in the high-level task of identifying and localizing an entire object as a gestalt. This dilemma has been, and continues to be, a fundamental unmet challenge in image segmentation.

In this disclosure, we propose a unique approach to mitigate the above challenge by considering segmentation as consisting of two dichotomous processes—recognition and delineation. Recognition is the process of finding the whereabouts of the object in the image, in other words, localizing the object. Delineation is the process of precisely delineating the region occupied by the object.

Recognition is a high-level process. It is trivial for knowledgeable humans to recognize objects in images. Yet, delineation is a meticulous operation that requires low(pixel)-level detailed quantitative information. For knowledgeable humans, it calls for toilsome effort, which makes manual object delineation impractical as a routine approach. However, computer algorithms, particularly DLNs, can outperform humans in reproducibility and efficiency of delineation once accurate recognition help is offered to them. This disclosure synergistically marries strengths in recognition coming from natural intelligence (NI) or human knowledge with the unmatched capabilities of DLNs (Artificial Intelligence, AI) in delineation to arrive at an integrated, robust, accurate, general, and practical system for medical image segmentation. This Recognition-Delineation (R-D) paradigm manifests itself at different levels within the proposed AAR-DL methodology as described below.

### Methods

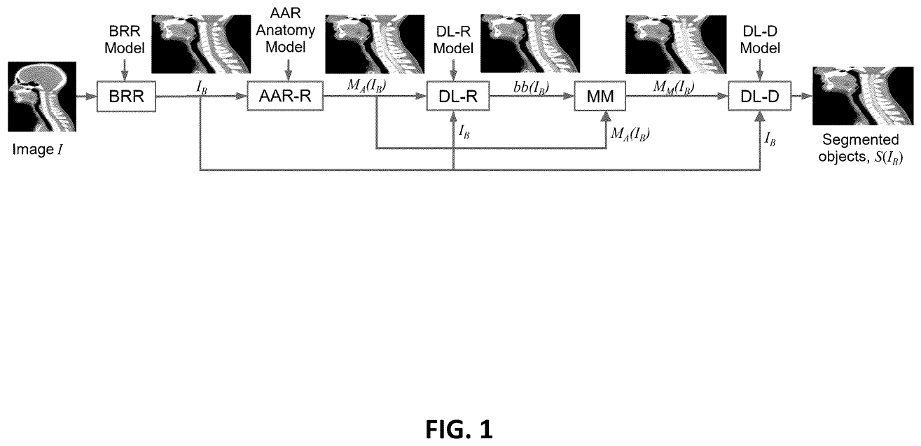

A schematic representation of the AAR-DL invention is shown in FIG. 1. There are four key stages in the whole methodology: BRR, AAR-R, DL-R, and DL-D. The first three relate to recognition and the fourth pertains to delineation in the spirit described above. In this disclosure, we will focus only on the object recognition aspect. The DL-D module may be substituted by any decent DL-based delineation engine. The protection sought in this disclosure is for the recognition component as described below. The goal of the three recognition components is to localize the object in the given image as precisely as possible so a DL-based method (4th component, DL-D) may perform delineation most accurately focusing only on the region indicated by the recognition steps. This allows DL-D to fully exploit its innate strengths rather than losing its specificity if it were to operate on the entire image to delineate the object.

This disclosure involves two key innovations, both pertaining to object recognition: (i) The R-D paradigm itself, integration of recognition and delineation engines, and successive refinement of recognition itself following the principles underlying the R-D paradigm, (ii) A new DL-based recognition refinement method that takes as input the recognition result from AAR-R and significantly improves upon AAR-R's accuracy following the R-D paradigm. These innovations are described below.

### (i) The R-D Paradigm, Integration of Recognition and Delineation, and Successive Refinement of Recognition Itself Following the Principles Underlying the R-D Paradigm

The R-D paradigm entails proceeding from global concepts to local concepts in stages where knowledge about global concepts is provided through NI and local concepts are handled via DL (AI). The global knowledge derived from NI and encoded into the AAR-DL methodology at various stages acts like a proxy to a medical expert such as a radiologist. At the BRR stage, global knowledge is imparted through a precise definition of the body region in terms of the superior and inferior anatomic extent [20, 52, 105]. The BRR algorithm then identifies the transaxial slices at these levels via DL by looking for anatomic details that are characteristically portrayed in those slices. If the input image fully contains multiple body regions, a trimmed 3D image is output for each identified body region. BRR recognizes a body region via a DL-network called BRR-Net [91] that is trained to identify the transaxial slices that constitute the superior and inferior boundaries of four body regions in the human torso—head & neck, thorax, abdomen, and pelvis. Among recognition modules (BRR, AAR-R, and DL-R), BRR represents the coarsest level, in the sense that the body region where the object of interest resides is found first. Recognition gets gradually refined as we proceed to other modules designated for recognition. BRR is based on the IP disclosure listed in Reference [106].

The second module AAR-R is based on the automatic anatomy recognition (AAR) methodology [30]. AAR performs object recognition driven by human knowledge (NI). It first creates an anatomy model from a set of images for which careful manual delineation of each object of interest in each image is given. Here again, precise anatomic definition is strictly followed for each object within each well-defined body region. In the anatomy model, the objects are arranged in a hierarchy where the geometric relationships among the objects are encoded. The anatomy model essentially represents codified human knowledge of anatomy of that body region. AAR-R performs object recognition in a given image by first placing the model of each object in the image guided by the anatomy model (via NI) and subsequently refines the placement (via AI) so that the model fits optimally the underlying image intensity pattern. Note how the R-D paradigm plays a role even in AAR-R. Implementation of the AAR-R module follows the patent that has been filed, Reference [105].

In this manner, the location of an object O gets refined successively from global to local level starting from the very gross body region level first where BRR finds the body region in which O resides. Then within the found body region, O is roughly identified by using the anatomy model. This constitutes the second level of refinement. In the third level, O's location is refined by AAR-R in the image by optimally fitting the model to the image intensity pattern. In the fourth and fifth levels, O's location and size are intimately refined via the DL-R module to output the final recognition result. Some details of the DL-R module are given below.

### (ii) A New DL-Based Object Recognition Refinement Method

This component is represented by the DL-R module in FIG. 1. Let O be the object being recognized in the given input image I and FM(O) denote the fuzzy model of O output by AAR-R for image I. DL-R takes as input: fuzzy model FM(O), image I, and the trained DL-R model. It outputs a stack of 2D rectangular bounding boxes where each 2D rectangle snugly encloses O in each slice of I. This constitutes the 4th level mentioned above. Finally, to this stack of 2D rectangles, the fuzzy model FM(O) is fitted, and this fitted fuzzy model is output by the module labeled Object Recognition in FIG. 1. This constitutes the 5th level.

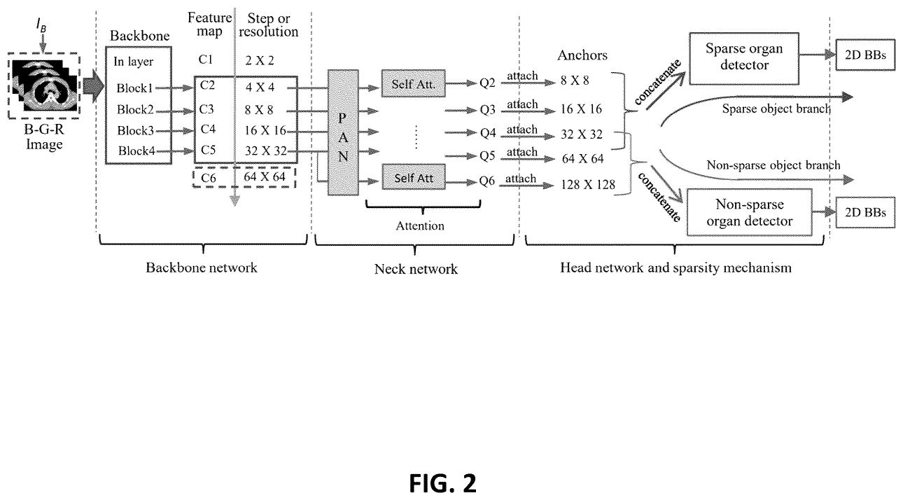

The architectural details of the DL-R module (see FIG. 2) are as follows. It comprises or consists of two separate CNN based DL networks—one for handling sparse organs (that is, small, tubular, or spatially sparse structures) and another for handling non-sparse organs (that is, large, space-filling, and blob-like structures). These networks are inspired by the architecture of RetinaNet and employ a Focal Loss function. The network structure consists of three parts: a backbone network—Resnet 50, a neck network—PathAggregation network (PAN), and a head network—anchor-based classification/regression network [107]. Some post-processing steps are subsequently applied to the stack of 2D rectangles including non-maximum suppression to remove overlapping bounding boxes and interpolation of bounding boxes between slices to fill any slices through O where bounding boxes have not been detected. A block diagram of the network architecture is shown in FIG. 2.

### Results

The R-D paradigm at the system level allows maximally exploiting the capabilities of NI and AI and to integrate them into a practical system. The principle underlying R-D continues at each individual sub-system level as explained above. As a result, the AAR-DL system as a whole becomes very robust and capable of accurately recognizing and delineating all types of objects—large space-filling and well-defined objects, small sparse objects, and objects with poor boundary definition and distorted by pathology, artifacts, and surgical manipulation.

The AAR-DL system was tested as follows on two body regions—thorax and head & neck (H&N). We utilized a set of 50 near-normal diagnostic computed tomography (CT) data sets of the thorax gathered from the patient image database of the Hospital of the University of Pennsylvania for creating AAR models involving 10 objects in the thorax. Similarly, utilizing a separate set of 40 diagnostic CT data sets of the H&N region, we created AAR models of the H&N region involving 16 objects. We gathered an additional 75 CT data sets for the thorax and 85 CT data sets for H&N from patients undergoing radiation treatment planning for cancer in the two body regions. This cohort of 125 data sets for each body region was used for training the BRR and DL-R networks separately for each body region. In this manner, 4 DL network models were created—BRR and DL-R models for thorax and BRR and DL-R models for H&N. An additional and separate set of 25 CT data sets of the thorax and 100 CT data sets of H&N were gathered for testing the AAR-DL system. These data sets again represented CT images acquired for planning radiation therapy of patients with cancer in the two body regions.

We created delineations of all objects in the testing data sets manually following the strict definitions of body regions and objects to help us determine the accuracy of the recognition result output by the DL-R module. We express accuracy of recognition via two metrics: (i) location error LE (in mm) indicating the distance between the geometric center of the ground truth object and the geometric center of the object model output by DL-R; (ii) scale error SE expressing the ratio of the size of the output object model to the size of the true object. The mean and standard deviation (SD) of LE and SE over the tested data sets are listed in Tables 1 and 2, respectively, for thorax and H&N body regions. Note that the ideal values for LE and SE are, respectively, 0 mm and 1.0.

### CONCLUSIONS

1. Over all objects (last column in Tables 1 and 2), the localization error for the AAR-DL system is within 6 mm for thorax (˜2 voxels, given that voxel size is ˜1×1×3 mm³), and within 5 mm for H&N ((˜2 voxels, given that voxel size is ˜1×1×2-3 mm³). Excluding a few very challenging objects (TEs, ePBT, and CPA in thorax and none in H&N), all other objects can be located within 1-2 voxels. This is remarkable, especially for objects with poorly expressed image boundaries like TEs, TSC, LCW, RCW, LPG, RPG, LSmG, RSmG, OHPh, CtEs, SpGLx, TG, Lp, LBMc, RBMc, and CtBrStm. We attribute this effectiveness to the innovations mentioned above.

2. Object delineation is not part of the innovation in this IP disclosure and delineation results are hence not shown here. With the accuracy achieved in DL-R for recognition, we have observed excellent accuracy for delineation wherein for most clinical CT studies in the radiation therapy (RT) application area, the delineation output by the AAR-DL system requires minor, if any, corrections for use of the output of AAR-DL for routine RT planning purposes.

3. The AAR-DL system particularly shows consistently outstanding performance on CT studies with pathology and artifacts and for objects with poor boundary contrast.

4. The methodology underlying AAR-DL can be easily extended to other body regions such as the abdomen and pelvis for RT and other non-RT applications by recreating models (AAR, DL-R, and DL-D models) appropriate for those body regions and the objects they contain. Furthermore, the methodology can easily be extended to other cross-sectional imaging modalities such as magnetic resonance imaging (MRI), positron emission tomography (PET), and single photon emission computed tomography (SPECT).

In RT planning, the goal is to maximize the radiation dose delivered to the tumor and avoid irradiating healthy critical organs in the vicinity of the tumor. Typically, RT planning is done by first obtaining a computed tomography (CT) scan of the cancer patient and segmenting or contouring the organs at risk (OARs) in the images. Currently, this is done mostly manually. Manual contouring is labor-intensive, time consuming, and prone to operator variability. Many methods are currently being developed using advanced artificial intelligence (AI) techniques. These competing methods all have the same drawback as we mentioned earlier, namely lack of high-level knowledge properly incorporated into the system so that DL-D operation can be confined to the vicinity of the OAR of interest. The innovations described above—the R-D paradigm, integration of recognition and delineation, successive refinement of recognition, and DL-based recognition refinement—allow the AAR-DL system to achieve high accuracy in contouring OARs, not only those with well-defined boundaries but also those with poor contrast, distortion by artifacts, or distortion by pathology and surgical manipulations. As a result, we claim that our system achieves the highest contouring efficiency by way of the least amount of post hoc manual corrections required for the output of AAR-DL for use in RT planning. This system can also be used for segmentation in other non-RT planning diagnostic imaging applications and in other non-CT cross-sectional imaging modalities such as magnetic resonance imaging (MRI), positron emission tomography (PET), and single photon emission computed tomography (SPECT).

TABLE 1
LE and SE (mean and SD) over the tested data sets for the thorax body region.

| Object | LLg | RLg | TEs | ePBT | TSC | LCW | RCW | Hrt | TAo | CPA | All |
|---|---|---|---|---|---|---|---|---|---|---|---|
| LE mm | 2.91 ± | 2.12 ± | 8.85 ± | 9.66 ± | 5.43 ± | 4.16 ± | 4.39 ± | 5.29 ± | 6.48 ± | 11.24 ± | 6.05 ± |
| (Mean, | 3.10 | 1.77 | 8.98 | 4.80 | 4.74 | 2.94 | 3.19 | 7.88 | 5.30 | 6.56 | 4.93 |
| SD) | | | | | | | | | | | |
| SE | 0.99 ± | 0.98 ± | 0.99 ± | 1.01 ± | 0.98 ± | 1.00 ± | 0.99 ± | 0.99 ± | 1.02 ± | 0.89 ± | 0.98 ± |
| (Mean, | 0.02 | 0.04 | 0.08 | 0.06 | 0.04 | 0.02 | 0.01 | 0.03 | 0.07 | 0.12 | 0.05 |
| SD) | | | | | | | | | | | |

LLg, RLg: Left & right lung; TEs: Thoracic esophagus; ePBT: Extended proximal bronchial tree; TSC: Thoracic spinal canal; LCW, RCW: Left & right chest wall; Hrt: Heart; Tao: Thoracic aorta; CPA: Central pulmonary arteries.

TABLE 2
LE and SE (mean and SD) over the tested data sets for the H&N body region.

| Object | CtSc | LPG | RPG | LSmG | RSmG | Mnd | OHPh | CtEs | SpGLx |
|---|---|---|---|---|---|---|---|---|---|
| LE | 7.08 ± | 3.99 ± | 4.99 ± | 5.68 ± | 4.67 ± | 6.30 ± | 6.29 ± | 6.06 ± | 2.99 ± |
| mm | 1.30 | 2.45 | 2.13 | 3.71 | 3.16 | 6.83 | 5.22 | 5.45 | 2.09 |
| (Mean, | | | | | | | | | |
| SD) | | | | | | | | | |
| SE | 1.02 ± | 0.89 ± | 0.90 ± | 0.78 ± | 0.84 ± | 0.94 ± | 0.96 ± | 1.01 ± | 0.95 ± |
| (Mean, | 0.04 | 0.08 | 0.09 | 0.14 | 0.12 | 0.06 | 0.13 | 0.17 | 0.08 |
| SD) | | | | | | | | | |

| Object | TG | eOC | Lp | LBMc | RBMc | CtTr | CtBrStm | All |
|---|---|---|---|---|---|---|---|---|
| LE | 4.96 ± | 2.91 ± | 5.23 ± | 5.18 ± | 7.28 ± | 5.97 ± | 4.36 ± | 5.24 ± |
| mm | 5.61 | 1.42 | 3.01 | 3.07 | 3.89 | 6.36 | 3.15 | 3.67 |
| (Mean, | | | | | | | | |
| SD) | | | | | | | | |
| SE | 0.98 ± | 0.98 ± | 0.94 ± | 0.80 ± | 0.80 ± | 1.06 ± | 0.87 ± | 0.92 ± |
| (Mean, | 0.14 | 0.03 | 0.07 | 0.18 | 0.13 | 0.11 | 0.11 | 0.10 |
| SD) | | | | | | | | |

CtSc: Cervical spinal cord; LPG, RPG: Left & right parotid glands; LSmG, RSmG: Left & right submandibular glands; Mnd: Mandible; OHPh: Orohypopharynx constrictor muscle; CtEs: Cervical esophagus; SpGLx: Supraglottic larynx; TG: Thyroid gland; eOC: Extended oral cavity; Lp: Lips; LBMc, RBMc: Left & right buccal mucosa; CtTr: Cervical trachea; CtBrStm: Cervical brain stem.

Additional Background: Automatic segmentation of 3D objects in computed tomography (CT) is challenging. Current methods, based mainly on artificial intelligence (AI) and end-to-end deep learning (DL) networks, are weak in garnering high-level anatomic information, which leads to compromised efficiency and robustness. This can be overcome by incorporating natural intelligence (NI) into AI methods via computational models of human anatomic knowledge.

Purpose: We formulate a hybrid intelligence (HI) approach that integrates the complementary strengths of NI and AI for organ segmentation in CT images and illustrate performance in the application of radiation therapy (RT) planning via multisite clinical evaluation.

Methods: The system employs five modules: (i) body region recognition, which automatically trims a given image to a precisely defined target body region; (ii) NI-based automatic anatomy recognition object recognition (AAR-R), which performs object recognition in the trimmed image without DL and outputs a localized fuzzy model for each object; (iii) DL-based recognition (DL-R), which refines the coarse recognition results of AAR-R and outputs a stack of 2D bounding boxes for each object; (iv) model morphing (MM), which deforms the AAR-R fuzzy model of each object guided by the output from DL-R; and (v) DL-based delineation (DL-D), which employs the object containment information provided by MM to delineate each object. NI from (ii), AI from (i), (iii), and (v), and their combination from (iv) facilitate the HI system.

Results: The HI system was tested on 26 organs in neck and thorax body regions on CT images obtained prospectively from 464 patients in a study involving four RT centers. Data sets from one separate independent institution involving 125 patients were employed in training/model building for each of the two body regions, whereas 104 and 110 data sets from the 4 RT centers were utilized for testing on neck and thorax, respectively. In the testing data sets, 83% of the images had limitations such as streak artifacts, poor contrast, shape distortion, pathology, or implants. The contours output by the HI system were compared to contours drawn in clinical practice at the four RT centers by utilizing an independently established ground-truth set of contours as reference. Three sets of measures were employed: accuracy via Dice coefficient (DC) and Hausdorff boundary distance (HD), subjective clinical acceptability via a blinded reader study, and efficiency by measuring human time saved in contouring by the HI system. Overall, the HI system achieved a mean DC of 0.78 and 0.87 and a mean HD of 2.22 and 4.53 mm for neck and thorax, respectively. It significantly outperformed clinical contouring in accuracy and overall saved 70% of human time over clinical contouring time, whereas acceptability scores varied significantly from site to site for both auto-contours and clinically drawn contours.

Conclusions: The HI system is observed to behave like an expert human in robustness in the contouring task but vastly more efficiently. It seems to use NI help where image information alone will not suffice to decide, first for the correct localization of the object and then for the precise delineation of the boundary.

### 1|Introduction

### 1.1|Background

The focus of this paper is on segmenting 3D objects in medical computed tomography (CT) images, and the application area considered is the segmentation of objects for radiation therapy (RT) planning. “Objects” in this context refer to body regions (e.g., neck and thorax) and organs (e.g., heart and lungs). Although the methods described in this paper are applicable to other “objects” such as tissue regions and lymph node zones, our illustrations in this paper will be via body regions and organs. A fundamental design strategy in our approach is that we formulate image segmentation as two sequential and dichotomous operations-object recognition and object delineation. Recognition (R) is the process of determining the whereabouts of the object in the image. Delineation (D) performs precise contouring of the object boundary or delineation of the region occupied by the object. On the one hand, recognition is a high-level process. Knowledgeable humans perform this task instantaneously and with ease and robustness, whereas it is much harder for machine algorithms. On the other hand, reproducible, efficient, and precise delineation is much harder for humans to perform than for machine algorithms, especially once object location information is provided to the machine via recognition.

In this paper, these complementary traits of natural intelligence (NI) of human experts versus artificial intelligence (AI) of computers embedded in algorithms constitute the central thread. Around this thread, we synergistically weave prior high-level knowledge coming from human experts with the unmatched capabilities of deep learning (DL) algorithms to meticulously harness and utilize low-level details. The resulting hybrid-intelligence (HI) methodology presented in this paper overcomes crucial gaps that currently exist in state-of—the-art DL algorithms for medical image segmentation.¹ This, we believe, brings us close to a breakthrough in anatomy segmentation.

Literature on general image segmentation dates back to the early 1960s.^{1,2} Principles for medical tomographic 3D image segmentation began to appear in the late 1970s^{3,4} with the availability of CT images. Approaches to medical image segmentation can be classified into two broad groups: purely image-based and prior-knowledge-based. Purely image-based approaches make segmentation decisions based entirely on information derived from the given image.⁵⁻²¹ They predate prior-knowledge-based-approaches and continue to seek new frontiers. In prior-knowledge-based approaches,²²⁻⁵¹ known object shape, image appearance, and object relation information over a subject population are first codified (modeled/learned) and then utilized on a given image to bring constraints into the segmentation process. They evolved to overcome failure of purely image-based approaches due to inadequate image information and, equally importantly, for automation. Among prior knowledge-based approaches, three distinct classes of methods can be identified-object-model based,²²⁻³¹ atlas based,³²⁻⁴¹ and DL based.⁴²⁻⁵¹ Often, they are all called model-based as they all form some form of models of the prior knowledge. DL networks are also referred to as “models.” Nonetheless, there is a fundamental distinction among them—the former two are principally NI-driven, whereas the latter is chiefly AI-driven.

For the purpose of this paper, by NI we mean know-how about human anatomy, shape, and geographic layout of objects in the body, objects' appearance in images (such as their conspicuity and vulnerability to artifacts), compactness of shape/size (such as thin elongated vs. compact blob-like objects and small vs. large objects), and malleability in shape/size and position. The HI-methodology we propose encodes this know-how (NI) explicitly as computational models so as to provide recognition guidance for DL (AI) algorithms. In the recognition-delineation duo, called the RD-paradigm throughout, recognition (R) is facilitated mainly by the computational models encoding NI, and delineation (D) is driven by DL algorithms.

### 1.2|Related Literature

Although most segmentation methods implicitly perform some form of the R- and D-steps, these steps are not designed with the intentional and explicit use of NI and employing AI techniques as we described above. Live-Wire¹³ is the first work where this paradigm was identified explicitly, and the method was designed with separate R- and D-steps. It is an interactive slice-by-slice segmentation method where recognition is manual (aided by NI) and delineation is automatic (AI), and the two processes are seamlessly coupled in real time.

Prior knowledge-based approaches allowed encoding of prior knowledge and facilitated bringing this to bear on recognition automatically. Among these, object-model-based strategies²²⁻³¹ that explicitly encode object information in the model such as automatic anatomy recognition (AAR) methods of Refs. [30, 52-55] have advantages for recognition over other methods, such as those based on atlases, for two reasons: (i) They explicitly encode object information, including geographic layout and object relationships. Consequently, they can handle nonlinearities and discontinuities in object relationships in human anatomy that are known to exist.⁵⁶ Recognition strategies that rely on image registration used by atlas methods require smooth deformations that have difficulties in handling discontinuities and nonlinearities in object relationships/geographic layout adequately.

Machine learning methods for detecting bounding boxes (BBs) body-wide existed in medical imaging for some time.⁵⁷⁻⁶⁰ Recently, DL methods have evolved for this purpose (often under the name “attention mechanisms”) with improved results.⁶¹⁻⁷² These methods do not qualify as representatives of the R-step in the spirit of the RD-paradigm of the HI-strategy mentioned earlier for several reasons. (i) They require large swaths of annotated data sets, making them less efficient for training/model building (annotation time and computational time) compared to model-based methods such as Refs. [30, 52] that explicitly encode object information. (ii) They all detect 2D or 3D BBs to localize objects and are hence less specific than geometric and geographic model-based methods that output object-shaped containers² enclosing the objects of interest at the completion of the R-step. This has several repercussions. As 3D BBs are rectangular boxes, they invariably enclose other objects lying in the vicinity of the object of interest. This can cause the subsequent D-step to lose specificity and accuracy. This non-specificity can vary considerably depending on the orientation of the object, especially for elongated and spatially sparse objects. If 2D BBs are employed instead, although non-object information captured within the BBs can be minimized, a separate issue arises as to how to integrate all 2D BBs corresponding to the same 3D object accurately. (iii) In their current state of development, the BB methods do not permit direct and explicit incorporation of NI information into the segmentation process as in the HI-methodology.

Although some prior knowledge-based approaches exist, we do not believe they have exploited the full potential of this paradigm toward an HI-strategy to date. For example, in AAR,^{30,52-11} which is a fuzzy model-based method, although the R-step is fully automated and powerful, the D-step is suboptimal, especially in comparison to the power of current DL methods in delineation. In DL methods, the R-step (when it exists) is weak, as explained before. The goal of this paper is, therefore, to present an HI-strategy that employs recognition concepts coming from geometric and geographic model-based methods with the power of state-of-the-art DL methods in delineation to arrive at an anatomy segmentation methodology that is demonstrably on par with expert-level manual segmentation (recognition and delineation) in accuracy and robustness but completely automated. Among other recent examples of knowledge-based methods that employ AI techniques,⁷³⁻⁸⁰ Isensee et al.⁷³ proposed a two-stage coarse-to-fine segmentation method based on two 3D U-Nets. For the first stage, a down-sampled image is an input into a 3D U-Net to coarsely segment the target organ. Then after up-sampling of the output of the first stage, another 3D U-Net is applied to refine and achieve full resolution segmentation. Wang et al.⁷⁴ also proposed a two-stage segmentation method, which employs two dedicated 3D U-Nets for recognizing and delineating the objects separately. Men et al.⁷⁵ proposed a coarse-to-fine approach that consisted of a simple region detector and a fine segmentation unit. Tang et al.⁷⁶ employed the Region Proposal Network on 2D slices to localize the target organs and then used a 3D U-Net to perform delineation. Guo et al.⁷⁷ divide the ensemble of 42 objects into three levels-anchor, mid-level, and small and hard, and the RD-paradigm is applied only to the small and hard objects. Although these methods achieved impressive performance, none of them involved the NI-strategy for deployment of anatomic knowledge explicitly in method design.

Examples of papers that have roughly the same spirit as that of the HI-strategy we propose, yet that principally differ, are as follows. Kaldera et al.⁸¹⁻⁸³ applied the AI-strategy to detect the region of interest (ROI) of the object and then applied purely image-based methods to perform delineation. Zhao et al.⁸⁴ used a graph-based registration method to deploy an atlas in the R-step and then performed fine D-step based on the DL method. Lessmann et al.⁸⁵ proposed an iterative segmentation and identification method for vertebra on CT scans. This method segments one vertebra in each iteration and uses the previously segmented vertebrae to guide anatomy as anatomy for the next iteration. BB-U-Net proposed by El Jurdi et al.⁸⁶ embeds prior knowledge through a novel convolutional layer introduced at the level of skip connections in the U-Net architecture. Prior knowledge provided comes from manually input BBs. Other works similarly achieve fine segmentation through human interaction⁷⁻⁹⁰ by embedding NI in the segmentation procedures via manual operations. These interactive algorithms have reported impressive performances, but they need actions from experts during DL training and/or prediction.

### 1.3|Key Innovations

This work comprises four key innovations. (i) Standardized object definition: We introduced this concept previously³⁰ but here we exploit it to marry NI and AI innovatively. This concept allows us to encode and transfer prior anatomic knowledge to the AI algorithms at various stages remarkably effectively. It facilitates rough localization of all objects inexpensively, consistent with the mutual geographic bearing among all objects. (ii) RD paradigm: This concept represents a computational means of effectively supplying prior knowledge to the AI methods. Although earlier proposed under Live-Wire and other methods, here we take only the underlying principle and expand it to automatically transfer NI to AI techniques. (iii) Anatomy-guided DL: This factor constitutes the means of how to exploit prior knowledge once it is supplied into the DL methods. (iv) Model morphing (MM): This novel concept relates to an amalgamation of NI and AI by feeding back the DL local findings to the anatomy models. As will be seen, these four factors are interwoven.

### 2|Materials and Methods

### Notations and Terminology

We will describe some of the key global notations and terminology in this section. See FIG. 1 for a schematic layout of our HI system. The description of the notations is sufficient to understand the overall operations depicted in FIG. 1. Notations that are local will be explained at appropriate points in the later sections in our description.

B: body region of interest. We will focus on neck (Nk) and thorax (Th) body regions in this paper; the methodology itself is applicable to any body region

[symbol] : set of objects or organs of focus in B

I: image data sets of body region B. In this paper, we focus on computed tomography (CT) imagery

I: any acquired image of a body region B

I_{B}: acquired image I after it is trimmed to the body region B

M: the set {FM(O): O∈ [symbol] } (of fuzzy geometric models with one fuzzy model FM(O) for each object O∈ [symbol] . The fuzzy model FM(O) for O is built from a given set of images such as I and the associated set of ground-truth (GT) binary images for O.

FM_{O}(I_{B}): the fuzzy model FM_{O} adjusted to image I_{B} by the AAR recognition process AAR-R

M_{A}(I_{B}): the set {FM_{O}(I_{B}): O∈ [symbol] } of fuzzy models FM_{O}(I_{B}) adjusted by the AAR-R process to the trimmed image I_{B}

bb_{O}(I_{B}): the stack of 2D BBs for object O∈ [symbol] representing the output of the deep learning recognition module DL-R for I_{B} for O

$$bb ( {I}_{B} ) := \{ {bb}_{O} ( {I}_{B} ) : O ∈ 𝒪 \}$$

FM_{O,M}(I_{B}): the output of the model morphing module MM upon morphing the fuzzy model FM(O) of 0 guided by the BBs contained in bb_{O}(I_{B})

$${M}_{M} ( {I}_{B} ) := \{ {FM}_{O , M} ( {I}_{B} ) : O ∈ 𝒪 \}$$

S_{O}(I_{B}): binary segmented image finally output by the HI system for object O∈ [symbol] in I_{B}

$$S ( {I}_{B} ) := \{ {S}_{O} ( {I}_{B} ) : O ∈ 𝒪 \}$$

NI: natural intelligence

AI: artificial intelligence

HI hybrid intelligence

GT: ground truth

DL: deep learning

AAR: automatic anatomy recognition

RD paradigm: recognition-delineation paradigm

ROI: region of interest

BB: bounding box

RT: radiation therapy

FIG. 1 shows a schematic representation of the different stages (modules) in the proposed hybrid intelligence (HI) system. AAR-R, automatic anatomy recognition object recognition; BRR, body region recognition; DL-D, deep-learning-based object delineation; DL-R, deep-learning-based object recognition; MM, model morphing

DL: deep learning

AAR: automatic anatomy recognition

RD paradigm: recognition-delineation paradigm

ROI: region of interest

BB: bounding box

RT: radiation therapy

### 2.1|Overview of the HI System

Our HI-segmentation system that follows the RD-paradigm is schematically depicted in FIG. 1. It consists of five stages wherein object recognition information gathered from the given image I progresses gradually from global levels in the first stage to more local levels until the fourth stage. The first four modules perform/refine some form of object recognition and the fifth module accomplishes object delineation. At the outset, we formulate a precise definition of the body region and all anatomic objects of interest in it. The definitions are implemented precisely for creating the models needed for the five modules. Modules labeled “body region recognition (BRR)”,⁹¹ AAR object recognition (AAR-R),³⁰ and DL-D⁹² are based on earlier works that underwent major modifications to be integrated into the HI system. A very preliminary version of the DL-R method was presented at the 2022 SPIE Medical Imaging conference whose proceedings contained a brief description of the method and a preliminary evaluation of the DL-R process for recognizing objects in the neck body region. In the present paper, we integrate all modules in the full spirit of the RD-paradigm and evaluate the resulting complete HI system comprehensively at four RT centers for auto-contouring of organs at risk (OARs) in neck and thorax body regions by utilizing the simulation CT images of patients undergoing routine radiation treatment at those centers.

The first module BRR is a DL-network that performs BRR.⁹¹ Given an image I, it trims I to the axial superior and inferior boundary as per the definition of body region B in the cranio-caudal direction and outputs a trimmed image I_{B}. The second module AAR-R is a purely geometric and geographic model-based recognition module and does not involve DL. Following AAR principles,³⁰ it performs recognition of objects one-by-one following an optimal hierarchical arrangement of the objects. In this process, it makes use of precise object relationship information encoded in the model. This module outputs, for each object O, a fuzzy model FM_{O}(I_{B}) that is adjusted optimally to I_{B}, and a set M_{A}(I_{B}) of such models for all objects. DL-R is a DL-based recognition refinement system comprising two DL-networks-one each for handling sparse objects and non-sparse objects. It makes use of the region-of-interest (ROI) information contained in the fuzzy models FM_{O}(I_{B}) and finds 2D BBs bounding each object O slice-wise in I_{B} and outputs a stack bb_{O}(I_{B}) of 2D BBs for each object O. The fourth module MM deforms the fuzzy model FM_{O}(I_{B}) of each 0 guided by the stack of BBs bb_{O}(I_{B}) and outputs a modified set of models M_{M}(I_{B}). This step thus performs an amalgamation of NI with the information derived from AI. Finally, DL-D is a DL-based delineation system comprising a DL-network for each object. It employs the object containment information provided in M_{M}(I_{B}) to delineate each object O and to output a binary image S_{O}(I_{B}) and the set of delineations S(I_{B}) for all objects.

The following sections describe the five modules in turn in Sections 2.3-2.7.

### 2.2|Standardized Definition of Objects

Defining objects in a consistent and standardized manner has three key benefits: (i) Via standardized definition^{30,52} and anatomic analytics,⁵⁶ we can gamer and then encode extensive anatomic and geographic (object layout) knowledge into models that helps developing consistent, sharp, and accurate priors and making object recognition, hence, segmentation, more accurate. (ii) It makes object-specific measurements meaningful. (iii) It facilitates standardized exchange, reporting, and comparison of image analysis methods, clinical applications, and results. The body region outer skin contour is usually the most conspicuous and easiest object to segment. Once a body region is properly defined and the skin object enveloping it is segmented, this reliable entity can be used as an anchor/reference to roughly localize all objects in its interior with the help of the geographic AAR model. See Appendix A.1 for details on how we arrive at definitions and then create high-quality ground-truth (GT) delineations of objects.

Note that formulations in this section pertain to NI, keeping in mind the feasibility of codifying this knowledge in the form of models and bringing that knowledge explicitly into AI methods.

### 2.3|Body Region Recognition (BRR)

The BRR module takes as input a CT scan image I of a body region B (neck or thorax) and outputs an image I_{B} that is trimmed automatically to B by recognizing the axial slices corresponding to the superior and inferior boundaries of B. For this purpose, we adopted a previously developed DL network called BRR-Net⁹¹ whose aim was to detect superior and inferior boundary slices of four body regions-neck, thorax, abdomen, and pelvis—in whole-body positron emission tomography (PET)/CT studies. In this work, we adapted BRR-Net to operate on body region-specific diagnostic CT scans.

### 2.4|Object Recognition Via AAR (AAR-R)

The second module AAR-R (FIG. 1) takes the auto-trimmed image I_{B} as inputs. It recognizes all objects in O by making use of the AAR model that has been created for B and outputs a fuzzy mask FM_{O}(I_{B}) fitted to I_{B} for each object O. AAR is a general approach based on fuzzy anatomy modeling, developed before the advent of DL techniques, for recognizing and delineating all objects in a body region. It consists of three stages-model building, object recognition, and object delineation. In the spirit of the RD-paradigm, we employ only the recognition capabilities of AAR in the HI system. Hence, here we will briefly summarize only the model building and recognition processes of AAR for completeness.

In the model building step, a fuzzy anatomy model FAM(B) of body region B is developed as a quintuple³⁰ FAM(B)=(H, M, ρ, λ, η). For this purpose, near-normal data sets are utilized, the idea being to encode in NI near-normal anatomy and its normal variations. The first element H in FAM(B) denotes a hierarchical arrangement of the objects in B. This arrangement is key to capturing and encoding detailed information about geographic layout of the objects. M is a set of fuzzy models, with one fuzzy model FM(O) for each object O in B. FM(O) represents a fuzzy mask indicating voxel-wise fuzziness of 0 over the population of samples of O used for model building. The third element ρ represents the parent-to-child relationship of the objects in the hierarchy, estimated over the training set population. λ is a set of scale (size) ranges, one for each object, indicating the variation of the size of each object. The fifth element η includes a host of parameters representing object appearance properties. FAM(B) is built from a set of good quality near-normal (model-worthy) CT images of B over a population. The underlying idea in the spirit of the central precept of the HI-approach is that only anatomy that is nearly normal can be modeled for the purpose of conveying NI knowledge to the AI algorithms.

The AAR recognition process (AAR-R in FIG. 1) outputs, for each object O in O, the localized fuzzy model mask FM_{O}(I_{B}) that represents a transformed version of the original model fuzzy mask FM(O) after the latter is optimally matched to image I_{B}. AAR-R first recognizes the skin object. This initializes the hierarchical recognition process. Subsequently, following the hierarchy H, for any object O, as the parent is already recognized and the parent-to-child relationship ρ is known, it first scales and places FM(O) in I_{B} based on just ρ. This placement (pose) is then refined by using a search strategy.³⁰ This final adjusted fuzzy model mask, denoted by FM_{O}(I_{B}), is output by AAR-R that will be used by the DL-R process as a container ROI.

Exemplar recognition results for AAR-R are shown in the next section for both body regions where the slice of the fuzzy mask FM_{O}(I_{B}) is overlaid on the slice of I_{B} for several challenging objects.

Parameters of the AAR-R module: There are no parameters to be manually set; all needed parameters are estimated from the training set of images automatically.

### 2.5|Object Recognition Refinement Via Deep Learning (DL-R)

The third module DL-R uses the trimmed image I_{B} and the set of fuzzy model masks M_{A}(I_{B})={FM_{O}(I_{B}):O∈ [symbol] } output by AAR-R to determine the set of stacks of 2D BBs bb(I_{B})={bb_{O}(I_{B}):O∈ [symbol] }, where the stack of snugly fit 2D BBs bb_{O}(I_{B}) for each object O is determined by making use of the fuzzy model mask FM_{O}(I_{B}) of that object output by AAR-R.

The DL network architecture designed for the DL-R module is shown in FIG. 2. The input image I_{B} is converted into a 3-channel image by dividing the CT gray scale into three component ranges-high attenuation (R), medium attenuation (G), and low attenuation (B).

The network consists of three subnetworks: backbone, neck, and head, as illustrated in FIG. 2. The input image is processed through the backbone network to output preliminary feature maps. In order to make full use of pretrained model weights, classical networks like ResNet and DenseNet are adopted in this work.C2-C5 denote the conv2, conv3, conv4, and conv5 output feature maps from the last layers of each backbone block. They have strides of 4, 8, 16, and 32 pixels, respectively, with respect to the input image. The neck network incorporates a Path Aggregation Network⁹³ followed by attention networks⁹⁴⁻⁹⁶ for producing feature maps with richer and more discriminating information. Finally, head networks with anchors predict location and category of target objects using 2D BBs in separate sparse-object and non-sparse-object branches. Anchors involve a set of preset areas and aspect ratios to improve the recognizing power of each candidate position for target objects. The anchors mechanism⁹⁷ enables the head network to be more adaptable to the target objects based on a limited number of prediction maps from the neck networks. Details for the backbone, neck, and head networks are provided in Appendix A.2.

FIG. 2 shows an overall architecture of the object sparsity-specific deep-learning (DL) recognition network employed by the deep-learning-based object recognition (DL-R) module

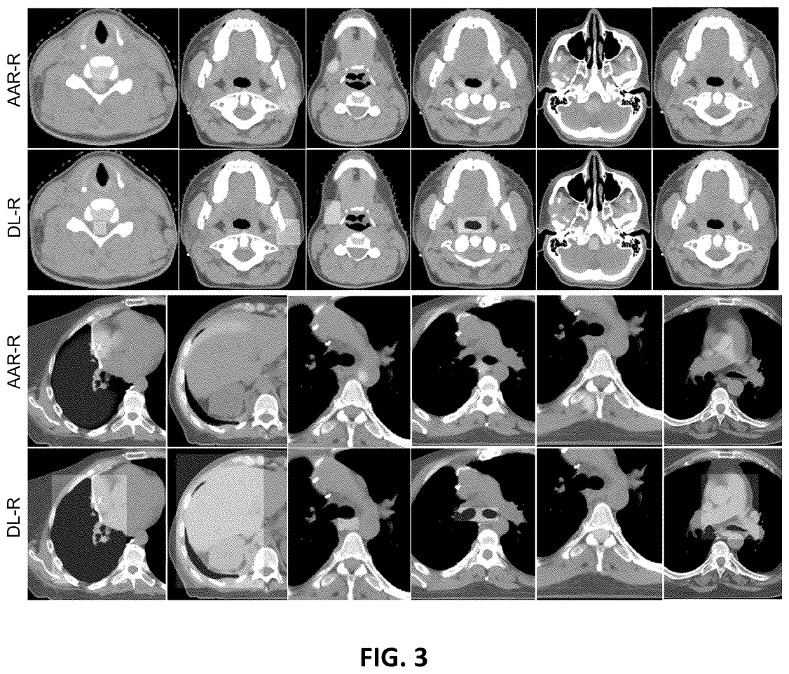

FIG. 3 illustrates the recognition process and the improvement achieved by the DL-R module over AARR for several challenging objects. AAR-R recognition results are also shown for reference.

Parameters of the DL-R module: The following hyperparameters are involved in DL-R model building. They influence the efficiency of model building and affect the model's ability to accurately perform recognition. Batch size=8. The number of training epochs for both non-sparse/sparse models=35. Steps in each epoch=5000. We apply the Adam optimizer with an initial learning rate of 1 e-5. Due to the use of a single GPU to train the model, considering the training speed and effect, the batch size is set to 8. The training process usually converges before 30 epochs. Experimentally, the training process of DL-R model is relatively stable, and reasonable changes of the hyperparameter will not affect the model performance significantly. The 512×512 slices of the given image are used as input to the DL-R module without resampling.

### 2.6|Object Model Morphing Guided by Bounding Boxes (MM)

AAR models are population 3D models and as such cannot match the detailed intensity intricacies seen in the input image I_{B}. In contradistinction, the DL-R process is exceptional in matching the intricacies but runs into difficulty when the details are compromised due to different anatomic appearances (such as open or closed lumens), artifacts, pathology, or posttreatment changes. The idea of combining via MM the information present in FM_{O} (_{B}) output by AAR-R and DL-R's output bb_{O} (_{B}) is to merge the best evidence from the two sources to create the modified fuzzy model FM_{O,M}(I_{B}) and the set of all models M_{M}(I_{B}) {FM_{O,M}(I_{B}):O∈ [symbol] }. This morphed model is expected to “agree” with DL-R output bb_{O}(I_{B}).In this stage, AI helps enrich NI by improving the anatomy model found by AAR-R.

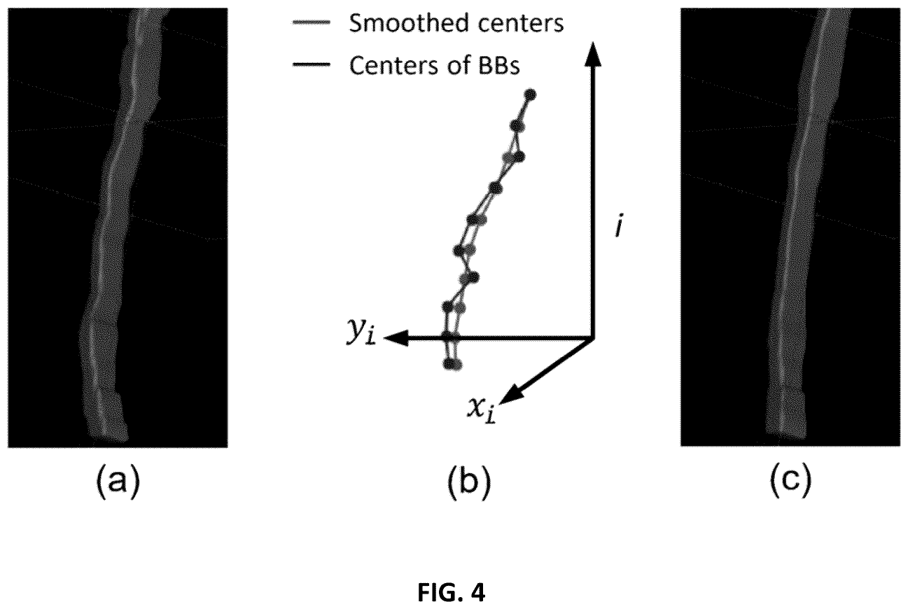

The morphing process in MM proceeds as follows. First, the geometric center of each 2D BB in bb_{O} (_{B}) is found in each slice. Subsequently, to the points so found over the slices occupied by O, a smooth curve is fit (see FIG. 4) using a second-degree polynomial as a function of the z-coordinates of the points by minimizing the mean-squared error. Subsequently, the fuzzy mask in each slice of FM_{O,M}(I_{B}) is then shifted so that the center of the fuzzy object in the slice is moved to the new center on the smoothed line. Further details of the morphing process can be found in Appendix A.3.

Parameters of the MMImodule: There are no parameters to be manually set; all needed parameters are estimated in the MM process itself.

### 2.7|Object Delineation Via Deep Learning (DL-D)

The last module DL-D makes use of the morphed recognition model FM_{O,M} (_{B})∈M_{M} (_{B}) of each object localized accurately in the trimmed image I_{B} to produce the final delineations S_{O}(I_{B})∈S(I_{B}). This module is a modified version of a previously designed DL network named ABCNet⁹² whose aim was to quantify body composition tissues, including subcutaneous adipose tissue, visceral adipose tissue, skeletal muscle, and skeletal tissue, in whole-body PET/CT studies without anatomy guidance from NI. The advantages of this DL-D model are the high efficiency on both computation and memory and its flexibility for object delineation in a 3D image. The model employs a typical residual encoder-decoder type of CNN but with some enhancements. In this network, the Bottleneck and feature map recomputing techniques are widely utilized. It can achieve a very deep structure, a large receptive field, and therefore an accurate segmentation performance even for these objects of very complex shape and confounding appearances, but with a relatively low number of parameters.

FIG. 3 is an illustration of the deep-learning-based object recognition (DL-R) recognition process and the improvement achieved over automatic anatomy recognition object recognition (AAR-R). AAR-R output is shown as a cross section of the fuzzy mask FM_{O}(I_{B}) overlaid on the computed tomography (CT) slice. DL-R output bb_{O}(I_{B}) is shown as a 2D bounding box (BB) next to the AAR-R result. Objects shown are (left to right and top to bottom): Neck: cervical spinal cord (CtSCd), left parotid gland (LPG), right submandibular gland (RSmG), orohypopharynx constrictor muscle (OHPh), cervical brain stem (CtBrStm), and left buccal mucosa (LBMc). Thorax: right lung (RLg), right chest wall (RCW), thoracic esophagus (TEs), extended proximal bronchial tree (ePBT), thoracic spinal canal (TSC), and central pulmonary artery (CPA)

We made several changes to adapt the architecture of ABCNet to our application of delineating organs. Instead of dynamic soft Dice loss, we directly employ classic Dice loss as FM_{O,M}(I_{B}) has already provided the ROI of the target object O to solve the imbalance problem between foreground and background. Furthermore, DL-D is a dichotomous segmentation model that can concentrate on delineating just the target object and not multiple objects simultaneously, making dynamic Dice loss less critical. Another change is that 3D patches with fixed size (72×72×72 voxels) are randomly selected within and around O and not in the whole image I_{B} to train the DL-D model. This is because FM_{O,M}(I_{B}) via our HI-strategy can provide accurate ROI information, and as such, features in the image space far away from O are unnecessary to be learned by the model. Before performing training and delineation, I_{B} is normalized by the zero-mean normalization method wherein the required means and standard deviations (SDs) are estimated from just within the ROI only. While training, the FM_{O,M}(I_{B}) of each O is simulated by a 3D dilation of the GT of O. This increases the specificity of normalization to just the object of interest, which is again facilitated by the HI-strategy.

FIG. 4 is an illustration of the smoothing process to obtain the modified fuzzy model FM_{O,M}(I_{B}) from FM_{O}(I_{B}). The exemplar object is thoracic spinal canal. (b) The process of obtaining the smoothed centers (red) from the centers computed from bb_{O}(I_{B}). (a) and (c) Surface renditions of bb_{O}(I_{B}) before and after smoothing

Although FM_{O,M}(I_{B}) can provide a smooth and accurate recognition mask of O, the input for DL-D are still 3D patches, which means that the patches may contain non-object-related areas from I_{B}. To address this problem, FM_{O,M}(I_{B}) is employed once again in postprocessing. As it is a smooth object-shaped container, FM_{O,M}(I_{B}) helps to remove false positives that lie far away from O in S_{O}(I_{B}). See examples of delineation shown in Section 3.

Parameters of the DL-D module: The networλ is trained for 10,000 iterations (50 epochs), with a batch size of 8. The initial learning rate is 0.01, which is reduced by the cosine annealing strategy for each epoch with a minimum learning rate of 0.00001. Different from the fixed input size strategy in the training stage, during inference, the complete 3D ROI of each O is directly input into the DL-D module. The 512×512 slices of the given image are used as input to the DL-R module without resampling.

### 2.8|Experiments

Evaluation of the HI system was performed via a prospective multicenter clinical study conducted following approval from the single central Institutional Review Board at the University of Pennsylvania. A waiver of consent and a waiver of Health Insurance Portability and Accountability Act (HIPAA) authorization were utilized in this study. Research study activities and results were not used for clinical purposes and did not affect/alter the clinical management of patients undergoing RT. Enrolled subjects did not undergo any research intervention in this study. The HI system was evaluated at four RT centers: University of Pennsylvania (Penn), New York Proton Center (NYPC), Washington University (WU), and Rutgers—The State University of New Jersey (RU).The study utilized data sets gathered at these centers from adult subjects with known cervical or thoracic malignancy who underwent simulation CT imaging, RT planning, and RT for clinical purposes.

The main goal of RT is ensuring that the proper radiation dose is delivered to tumor sites to maximize treatment effect while minimizing adverse effects related to radiation of healthy organs called OARs. Contouring of OARs and target tumor sites in medical images is required for these goals to be realized in clinical practice and is currently performed mostly manually or by using software that requires manual assistance. This is labor-intensive and prone to inaccuracy and inter-dosimetrist variability. In our evaluation of the HI system, we focus on auto-contouring of the OARs (and not of the tumor sites). Adult patients anticipated to undergo or already undergoing RT for treatment of cervical or thoracic malignancy (including planning or replanning CT imaging and contouring) were included in this study. Our evaluation goal was to compare the HI system to the methods currently used in clinical practice at these centers based on three factors of segmentation efficacy: accuracy, acceptability, and efficiency. Accuracy pertains to the agreement of the contours output by the HI system with an independently established GT segmentation, assessed via standard metrics like Dice coefficient (DC) and Hausdorff boundary distance (HD). Acceptability expresses expert evaluators' degree of subjective agreeability of the output of the HI system in comparison to the way they would draw contours in clinical practice. Efficiency of the HI system relates to the human labor required for mending the contours output by the HI system, so that they become acceptable for RT planning, in comparison to the human labor needed for current clinical contouring.

### 2.8.1|Acquiring Near-Normal Data Sets for AAR Model Building

The near-normal data sets required for AAR model building (Section 2.4) were selected by a board-certified radiologist (coauthor DAT) from the University of Pennsylvania Health System patient image database. See Appendix A.4 for details.

### 2.8.2|Acquiring Clinical Data Sets

Our goal was for each RT center to gather 30 scans for each body region (Neck and Thorax) from among its ongoing routine patient care cases with the following inclusion and exclusion criteria.

Inclusion criteria: (i) Age≥18 years. (ii) Known or suspected presence of cervical or thoracic malignancy. (iii) Anticipation to undergo or already undergoing/having undergone RT in the neck or thorax for clinical purposes. (iv) Anticipation to undergo or already having undergone CT imaging in the neck or thorax for clinical RT planning or replanning purposes.

TABLE 3
Key details pertaining to the clinical data sets used in this study

| Parameters | Neck | Thorax |
|---|---|---|
| Number of cases | 110 | 104 |
| Patient | Gender: male-92, female-18 | Gender: male-52, female-52 |
| demographics | Age: 18-50-17; 51-65-48; 66-80- | Age: 18-50-9; 51-65-22; 66-80-62; 81-95-11 |
| | 43; 81-95-2 | |
| | Race: White-90; African-14; Asian | Race: White-76; African-22; Asian-2; |
| | 4; unknown-2 | multiracial-3; unknown-1 |
| | Ethnicity: not Hisp/Latino-101; | Ethnicity: not Hisp/Latino-93; Hisp/Latino-6; |
| | Hisp/Latino-1; unknown-8 | unknown-5 |
| Patient condition | Cancer type: squamous cell carcinoma- | Cancer type: non-small cell lung cancer-59; small-cell |
| | 98; other-12 | lung cancer-6; metastatic melanoma-29; other-10 |
| | Prior radiation treatment: yes-12; no-98 | Prior radiation treatment: yes-33; no-71 |
| | Prior surgery: yes-52; no-56; anticipated | Prior surgery: yes-27; no-75; anticipated-1; |
| | 2 | unknown-1 |
| Scanner types | GE: Lightspeed 16-30 | GE: Lightspeed 16-29 |
| | Philips: Brilliance Big Bore-11; Gemini | Philips: Brilliance Big Bore-19; Gemini TF-20; |
| | Big Bore-16 | Gemini Big Bore-1 |
| | Siemens: Biograph mCT Flow-19; Somatom | Siemens: Biograph mCT Flow-13; Somatom Definition |
| | Definition Edge-12; Somatom | Edge-8; Somatom Confidence RT Pro-11; |
| | Confidence RT Pro-19; unknown-3 | unknown-3 |
| Image parameters | Contrast-enhanced: yes-70; no-40 | Contrast-enhanced: yes-35; no-69 |
| | Image size: 512 × 512 × [111-337] | Image size: 512 × 512 × [88-340] |
| | Pixel size: 0.98-1.27 mm | Pixel size: 0.98-1.60 mm |
| | Slice spacing: 1.5-3 mm | Slice spacing: 1.5-3 mm |

Exclusion criteria: (i) Age<18 years. (ii) No known or suspected presence of cervical or thoracic malignancy. (iii) No anticipation to undergo and not already undergoing/having undergone RT and CT imaging in the neck or thorax for clinical purposes. (iv) Subjects may be excluded at the discretion of the study Principal Investigator or other study team members based on the presence of other medical, psychosocial, or other factors that warrant exclusion.

Subject accrual was impacted by the Covid-19 pandemic such that not all sites were able to achieve the goal of 30 cases for each body region. To test and establish our system's generalizability, our goal was to challenge the HI system with data sets obtained by considering variations that occur in routine clinical practice in key parameters including (i) patient demographics: gender, age, ethnicity, and race; (ii) patient condition: cancer type and prior treatment (RT or surgery); (iii) scanner brands/models; and (iv) imaging parameters. Table 3 summarizes key details pertaining to these variables for the two body regions. (v) Additionally, only the near normal data sets and patient data sets acquired at Penn previously that are completely independent of the four clinical data sets mentioned previously were used for all model building and training operations.

### 2.8.3|Ground-Truth Segmentations, OARs, Training, and Testing Data Sets

Independent GT segmentations were created for both near-normal data sets (Table 15) used for model building and clinical data sets (Table 3) by following the procedure described in Section 2.2. In addition to these data sets in Tables 15 and 3, we utilized additional data sets (with GT available) from our previous study in Ref. [52] for both body regions. These data sets also pertained to cancer patients undergoing RT planning and were all from Penn. Table 4 lists the OARs considered in our system and their abbreviations, the number of data sets used for model building, the number of data sets used for training DL-R and DL-D networks, and the number of test samples used for evaluation. Given the large number of data sets and OARs, although GT contours (GC) were created for all data sets (except for very few cases where the OARs were partially or fully removed via surgery), clinically drawn contours were created for selected OARs, as commonly done in practice. The OARs and the number of samples selected for each OAR varied among sites. We note that seven dosimetrists and eight radiation oncologists were involved in creating clinical contours (CCs) at the four sites. Table 4 also lists the number of samples available for each OAR for the auto-contours (AC) output by our HI system.

TABLE 4
Organs at risk (OAR) considered in the two body regions for
evaluating the hybrid intelligence (HI) system and their abbreviations

| | | | | | |
|---|---|---|---|---|---|
| | | Number of | | | Number of |
| OAR | Abbreviation | test samples | OAR | Abbreviation | test samples |

Neck: AAR model building-39; DL-R and DL-D training-125 (39 used for AAR model
building + 86 from previous data set⁵²); testing-110 (from Table 3)

| | | | | | |
|---|---|---|---|---|---|
| Cervical brainstem | CtBrstm | 107 | Orohypopharyngeal | OHPh | 109 |
| | | | constrictor muscles | | |
| Cervical esophagus | CtEs | 107 | Supraglottic larynx | SpGLx | 107 |
| Cervical spinal cord | CtSCd | 109 | Extended oral cavity | eOC | 108 |
| Right submandibular | RSmG | 80 | Right buccal mucosa | RBMc | 90 |
| gland | LSmG | 81 | Left buccal mucosa | LBMc | 71 |
| Left submandibular | Mnd | 109 | Lips | Lp | 104 |
| gland | | | | | |
| Mandible | RPG | 105 | Cervical trachea | CtTr | 109 |
| Right parotid gland | LPG | 101 | Thyroid gland | TG | 97 |
| Left parotid gland | | | | | |

Thorax: AAR model building-58; DL-R and DL-D training-125 (58 used for AAR model
building + 67 from previous data set⁵²); testing-104 (from Table 3)

| | | | | | |
|---|---|---|---|---|---|
| Central pulmonary | CPA | 100 | Left lung | LLg | 100 |
| arteries | Hrt | 102 | Thoracic esophagus | TEs | 101 |
| Heart | RCW | 102 | Thoracic spinal | TSC | 102 |
| Right chest wall | LCW | 102 | canal Thoracic aorta | TAo | 102 |
| Left chest wall | RLg | 102 | Proximal bronchial | PBT | 102 |
| Right lung | | | tree | | |

Note:
The number of samples available for testing the system for each OAR is also shown.
Abbreviations:
AAR, automatic anatomy recognition;
DL-D, Deep-learning-based object delineation;
DL-R, deep-learning-based object recognition

### 2.9|Evaluation of the HI System

### 2.9.1|Assessment of Accuracy

We will use abbreviations GC—for independently established GT contours; CC—for clinically drawn contours; AC—for contours output by the automated HI system; and manually edited auto-contours (mAC)—for contours output by the HI system that have been mended manually by experts to make them acceptable. In RT applications, segmentations of an object are commonly referred to as “contours.” We will assume both terms segmentation and contour to mean a binary segmentation denoting the region occupied by the object and not its boundary as implied by the term “contour” and use the two terms interchangeably. For any patient image I, we will denote the corresponding ground truth, clinical, HI system output, and mended HI system output contours by I_{GC}, I_{CC}, I_{AC}, and I_{mAC}, respectively. The body region and the object under consideration will be clear from the context in which these variables are used.

For assessing accuracy, our goal is to compare the HI system output I_{AC}, its mended version I_{mAC}, and CC I_{CC} with GT I_{GC}. We will employ the commonly-used DC and HD as measures of accuracy:

$$DC ( {I}_{GC} , X ) = \frac{2 \vert {I}_{GC} ⋂ X \vert}{\vert {I}_{GC} \vert + \vert X \vert} , \qquad ( 1 )$$

where X=I_{CC} or I_{AC} or I_{mAC}. HD(I_{GC}, X) is defined as the mean of the mean boundary distances from I_{GC} to X and X to I_{GC}. As drawing CCs by experts is expensive, I_{CC} was created for a subset of the OARs and not all OARs considered in the two body regions. For the same reason, mending of the HI system contours by experts was also limited to a subset of the OARs. As such, the number of samples available for I_{GC} and I_{mAC} was lower than the number of samples of I_{AC} for both body regions.

### 2.9.2 Assessment of Acceptability

It is well known that DC and HD are not direct indicators of clinical “acceptability” of contours, and in particular that DC is sensitive to the size and spatial sparsity of objects.⁹⁸ Therefore, to study the clinical acceptability of contours independent of the previous metrics, we conducted a multicenter-blinded reader study involving 90 scans for each body region comprising 30 scans with GC, the same 30 scans with CC, and the same 30 scans with AC, all randomly mixed. These scans included scans with different image quality and OARs of different sizes and shapes, including five neck OARs: cervical spinal cord (CtSCd), right parotid gland (RPG), left submandibular gland (LSmG), mandible (Mnd), cervical esophagus (CtEs); and five Thorax OARs: right lung (RLg), thoracic esophagus (TEs), thoracic spinal canal (TSC), heart (Hrt), left chest wall (LCW). Each of the four sites conducted this reader study involving 90 scans where the reader assigned an acceptability score in a blinded manner on a 1-5 scale to each OAR sample: 1=extremely poor, not acceptable for RT planning; 2=poor, acceptable for RT planning after many modifications; 3=average, acceptable for RT planning after some modifications; 4=very good, acceptable for RT planning with very few modifications; 5=excellent, acceptable for RT planning with no or minimal modifications. Based on this score, we compared the three sets of contours, particularly CC and AC. We denote acceptability scores by AS(X) for X∈{GC, AC, CC}.

### 2.9.3|Assessment of Efficiency

The goal of efficiency assessment was to determine how much time the HI system would save compared to current clinical practice for contouring OARs. For each of the 30 scans for each body region, each of the four sites performed clinical contouring as they would normally do in their routine clinical practice and recorded the time taken by the dosimetrists/radiation oncologists for each OAR. The dosimetrists/radiation oncologists at the sites also performed adjustment of the HI system output as needed and recorded the required time for this adjustment. The two times were compared to ascertain the reduction in time due to the use of the HI system compared to the normal clinical process. The OARs considered for the efficiency study are as follows: neck: CtSCd, RPG, LSmG, Mnd, orohypopharyngeal constrictor muscles, CtEs, supraglottic larynx (SpGLx), extended oral cavity (eOC), cervical trachea (CtTr); thorax: RLg, TEs, extended proximal bronchial tree (ePBT), TSC, Hrt, LCW, and thoracic aorta (TAo). We will denote time taken for the two contouring processes by TC(X) for X∈{CC, mAC}.

The computational times for the various stages of the HI system in FIG. 1 were also assessed.

### 3|Results

Table 5 summarizes accuracy results for AC, CC, and mAC. Mean and SD values over the tested samples are listed. The number of test samples available for the three types of contours was not the same for reasons mentioned in Section 2.8. The total numbers (over all OARs) were, for neck: AC−1594, CC−1190, and mAC-943; for thorax: AC-1015, CC-728, and mAC-713. In the row labeled “All,” we list the mean and SD values of DC and HD over all OAR test samples. In the same row, we also list the p values of a paired t-test comparing AC to CC, AC to mAC, and CC to mAC for DC and HD.

TABLE 5
Accuracy results

| | DC(I_{GC}, X) | | | HD(I_{GC}, X) | | | | DC(I_{GC}, X) | | | HD(I_{GC}, X) | | |
|---|---|---|---|---|---|---|---|---|---|---|---|---|---|
| OAR | AC | CC | mAC | AC | CC | mAC | OAR | AC | CC | mAC | AC | CC | mAC |

Neck: Total number of test samples for: AC-1594, CC-1190, mAC-943

| | | | | | | | | | | | | | |
|---|---|---|---|---|---|---|---|---|---|---|---|---|---|
| Cervical | 0.74 | 0.44 | — | 2.91 | 12.02 | — | Orohypopharyn | 0.69 | 0.54 | 0.70 | 1.66 | 3.35 | 1.68 |
| brainstem | | | | | | | geal | | | | | | |
| | | | | | | | constrictor | | | | | | |
| | | | | | | | muscle | | | | | | |
| | 0.10 | 0.13 | | 3.07 | 4.34 | | | 0.09 | 0.13 | 0.07 | 1.68 | 1.99 | 1.02 |
| Cervical | 0.82 | 0.61 | 0.77 | 1.83 | 17.49 | 4.60 | Supraglottic | 0.85 | 0.53 | 0.73 | 1.22 | 5.09 | 2.97 |
| esophagus | | | | | | | larynx | | | | | | |
| | 0.09 | 0.11 | 0.09 | 1.37 | 14.02 | 5.87 | | 0.05 | 0.08 | 0.13 | 0.63 | 1.45 | 1.99 |
| Cervical | 0.82 | 0.67 | 0.80 | 1.90 | 13.33 | 2.72 | Extended oral | 0.89 | 0.58 | 0.74 | 2.74 | 10.3 | 7.17 |
| spinal cord | | | | | | | cavity | | | | | | |
| | 0.06 | 0.08 | 0.06 | 1.68 | 10.10 | 3.27 | | 0.07 | 0.16 | 0.23 | 2.43 | 26.26 | 7.59 |
| Right | 0.81 | 0.80 | — | 1.50 | 1.56 | — | Right buccal | 0.61 | 0.38 | — | 2.64 | 6.73 | — |
| submandibular | | | | | | | mucosa | | | | | | |
| gland | | | | | | | | | | | | | |
| | 0.13 | 0.16 | | 0.97 | 1.44 | | | 0.14 | 0.09 | | 2.00 | 2.22 | |
| Left | 0.81 | 0.76 | 0.80 | 1.83 | 2.61 | 1.57 | Left buccal | 0.61 | 0.39 | — | 2.51 | 6.37 | — |
| submandibular | | | | | | | mucosa | | | | | | |
| gland | | | | | | | | | | | | | |
| | 0.10 | 0.19 | 0.15 | 1.43 | 5.55 | 1.50 | | 0.15 | 0.11 | | 2.26 | 2.85 | |
| Mandible | 0.92 | 0.90 | 0.91 | 2.45 | 1.89 | 1.64 | Lips | 0.72 | 0.42 | — | 3.09 | 4.83 | — |
| | 0.04 | 0.05 | 0.03 | 2.19 | 1.38 | 1.28 | | 0.10 | 0.15 | | 1.46 | 1.81 | |
| Right | 0.78 | 0.80 | 0.80 | 2.92 | 2.39 | 2.48 | Cervical trachea | 0.80 | 0.73 | 0.85 | 1.79 | 4.34 | 2.16 |
| Parotid | 0.14 | 0.11 | 0.10 | 2.25 | 1.73 | 1.60 | | 0.07 | 0.09 | 0.06 | 1.10 | 3.65 | 1.43 |
| gland | | | | | | | | | | | | | |
| Left parotid | 0.80 | 0.80 | — | 2.63 | 2.46 | — | Thyroid gland | 0.81 | 0.78 | — | 1.78 | 1.77 | — |
| gland | | | | | | | | | | | | | |
| | 0.10 | 0.09 | | 1.75 | 1.41 | | | 0.14 | 0.08 | | 1.37 | 1.04 | |

| | | | | | | |
|---|---|---|---|---|---|---|
| All | 0.78 | 0.66 | 0.79 | 2.22 | 6.49 | 3.03 |
| | 0.13 | 0.19 | 0.13 | 1.83 | 7.70 | 4.01 |

Thorax: Total number of test samples for: AC-1015, CC-728, mAC-713

| | | | | | | | | | | | | | |
|---|---|---|---|---|---|---|---|---|---|---|---|---|---|
| Central | 0.79 | 0.58 | — | 4.76 | 13.72 | — | Left lung | 0.96 | 0.98 | — | 4.65 | 2.36 | — |
| pulmonary | | | | | | | | | | | | | |
| arteries | | | | | | | | | | | | | |
| | 0.10 | 0.22 | | 3.4 | 12.82 | | | 0.06 | 0.02 | | 6.28 | 2.04 | |
| Heart | 0.92 | 0.91 | 0.93 | 6.71 | 4.30 | 3.45 | Thoracic | 0.79 | 0.80 | 0.79 | 3.55 | 2.45 | 2.25 |
| | | | | | | | esophagus | | | | | | |
| | 0.06 | 0.04 | 0.03 | 9.95 | 2.33 | 2.84 | | 0.09 | 0.06 | 0.06 | 4.58 | 2.57 | 1.92 |
| Right chest | 0.82 | 0.57 | — | 4.70 | 8.52 | — | Thoracic | 0.89 | 0.74 | 0.86 | 2.53 | 19.69 | 2.44 |
| wall | | | | | | | spinal canal | | | | | | |
| | 0.06 | 0.08 | | 4.00 | 3.60 | | | 0.04 | 0.09 | 0.04 | 3.01 | 14.93 | 2.95 |
| Left chest | 0.83 | 0.55 | 0.80 | 4.07 | 9.21 | 3.88 | Thoracic aorta | 0.90 | 0.75 | 0.87 | 4.60 | 14.51 | 6.02 |
| wall | | | | | | | | | | | | | |
| | 0.06 | 0.09 | 0.07 | 2.57 | 5.95 | 2.30 | | 0.04 | 0.15 | 0.05 | 6.34 | 11.68 | 5.60 |
| Right lung | 0.97 | 0.98 | 0.96 | 5.61 | 2.40 | 2.10 | Proximal | 0.87 | 0.47 | 0.71 | 4.15 | 15.41 | 9.66 |
| | | | | | | | bronchial tree | | | | | | |
| | 0.02 | 0.02 | 0.01 | 11.20 | 1.46 | 1.72 | | 0.06 | 0.09 | 0.14 | 4.05 | 3.53 | 6.75 |

| | | | | | | |
|---|---|---|---|---|---|---|
| All | 0.87 | 0.79 | 0.85 | 4.53 | 8.00 | 4.26 |
| | 0.09 | 0.19 | 0.11 | 6.27 | 9.52 | 4.64 |

Abbreviation: AC, auto-contours; CC, clinical contour; DC, Dice coefficient; HD, Hausdorff distance; mAC, manually edited auto-contours; OAR, organ at risk; SD, standard deviation.
Note:
Mean and SD values of DC(I_{GC}, X) and HD(I_{GC}, X) for X ∈ {AC, CC, mAC} are shown.
p Value for DC: <0.001 for all three pair-wise comparisons
p Value for HD: <0.001 for all three pair-wise comparisons
p Value for DC: <0.001 for all three pair-wise comparisons
p Value for HD: AC to CC < 0.001; AC to mAC-0.47; CC to mAC < 0.001

In Table 6, we summarize acceptability study results AS(X) for the two body regions. For neck, some studies were not usable reliably for some OARs due to excessive artifacts or the data set not covering the full body region, and so on. This is the reason that the total number of test samples was 142 (<150=30 studies×5 OARs). In the row labeled “All,” we list the mean and SD values of AS(X) over all OAR test samples. In the next row, we list the p values of a paired t-test comparing GC to AC, GC to CC, and AC to CC for AS(X) values. Scores that are unusually low are highlighted in the table and explained in the next section.

TABLE 6
Acceptability results

| | Penn | | | NYPC | | | WU | | | RU | | |
|---|---|---|---|---|---|---|---|---|---|---|---|---|
| OAR | GC | AC | CC | GC | AC | CC | GC | AC | CC | GC | AC | CC |

Neck: Total number of test samples for GC-142, AC-142, CC-142

| | | | | | | | | | | | | |
|---|---|---|---|---|---|---|---|---|---|---|---|---|
| Cervical | 3.70 | 3.50 | 3.53 | 4.90 | 4.47 | 4.80 | 3.77 | 3.33 | 3.60 | 4.80 | 3.83 | 4.63 |
| spinal cord | | | | | | | | | | | | |
| | 0.75 | 0.63 | 1.04 | 0.31 | 0.68 | 0.48 | 0.77 | 0.71 | 0.89 | 0.76 | 1.21 | 0.49 |
| Right | 3.57 | 3.80 | 3.50 | 4.52 | 4.23 | 4.07 | 4.43 | 3.97 | 4.13 | 4.80 | 4.40 | 4.67 |
| parotid | | | | | | | | | | | | |
| gland | | | | | | | | | | | | |
| | 0.77 | 0.61 | 0.68 | 0.63 | 1.01 | 0.83 | 0.73 | 1.10 | 0.94 | 0.41 | 1.00 | 0.48 |
| Left | 4.00 | 4.23 | 4.03 | 4.75 | 4.53 | 4.68 | 4.52 | 4.23 | 4.20 | 4.80 | 4.66 | 4.80 |
| submandibular | | | | | | | | | | | | |
| gland | | | | | | | | | | | | |
| | 1.08 | 0.68 | 0.67 | 0.52 | 0.73 | 0.55 | 0.78 | 0.86 | 1.10 | 0.76 | 0.81 | 0.61 |
| Mandible | 3.80 | 3.77 | 3.83 | 4.83 | 4.63 | 4.47 | 4.83 | 4.33 | 4.20 | 5.0 | 4.77 | 4.87 |
| | 0.71 | 0.90 | 0.79 | 0.59 | 0.61 | 0.63 | 0.38 | 0.71 | 1.00 | 0.0 | 0.43 | 0.43 |
| Cervical | 4.03 | 3.83 | 3.93 | 4.63 | 4.40 | 4.77 | 4.77 | 4.30 | 4.27 | 4.93 | 4.43 | 4.83 |
| esophagus | | | | | | | | | | | | |
| | 0.67 | 0.75 | 0.83 | 0.67 | 0.86 | 0.57 | 0.43 | 0.79 | 0.94 | 0.25 | 0.77 | 0.38 |
| All | 3.82 | 3.83 | 3.77 | 4.73 | 4.45 | 4.55 | 4.46 | 4.03 | 4.08 | 4.87 | 4.42 | 4.76 |
| | 0.82 | 0.75 | 0.83 | 0.57 | 0.79 | 0.67 | 0.74 | 0.92 | 0.99 | 0.53 | 0.93 | 0.49 |

| | | | | |
|---|---|---|---|---|
| p Values | GC to AC: 0.94 | GC to AC: 0.09 | GC to AC: 0.00 | GC to AC: 0.00 |
| | GC to CC: 0.56 | GC to CC: 0.02 | GC to AC: 0.00 | GC to CC: 0.04 |
| | AC to CC: 0.50 | AC to CC: 0.21 | AC to CC: 0.61 | AC to CC: 0.00 |

Thorax: Total number of test samples for: GC-150, AC-150, CC-150

| | | | | | | | | | | | | |
|---|---|---|---|---|---|---|---|---|---|---|---|---|
| Right lung | 4.93 | 5.00 | 5.00 | 5.00 | 3.89 | 5.00 | 4.96 | 4.71 | 4.79 | 4.96 | 3.14 | 4.43 |
| | 0.38 | 0.0 | 0.0 | 0.0 | 1.03 | 0.0 | 0.19 | 0.76 | 0.50 | 0.19 | 1.56 | 0.9 |
| Thoracic | 5.00 | 4.79 | 5.00 | 4.61 | 3.89 | 4.82 | 4.68 | 4.59 | 4.68 | 4.50 | 2.75 | 4.61 |
| esophagus | | | | | | | | | | | | |
| | 0.0 | 0.69 | 0.0 | 0.50 | 1.20 | 0.39 | 0.77 | 0.89 | 0.72 | 0.64 | 1.32 | 0.57 |
| Thoracic | 5.00 | 5.00 | 5.00 | 4.96 | 4.70 | 4.89 | 4.96 | 5.00 | 4.96 | 4.86 | 4.67 | 4.43 |
| spinal | | | | | | | | | | | | |
| canal | | | | | | | | | | | | |
| | 0.0 | 0.0 | 0.0 | 0.19 | 0.82 | 0.31 | 0.19 | 0.0 | 0.19 | 0.36 | 0.73 | 0.84 |
| Heart | 4.79 | 2.89 | 5.00 | 4.86 | 2.61 | 4.07 | 4.68 | 4.46 | 4.39 | 4.07 | 1.82 | 3.61 |
| | 0.63 | 1.29 | 0.0 | 0.36 | 0.92 | 0.77 | 0.67 | 0.79 | 0.96 | 1.02 | 0.82 | 1.26 |
| Left chest | 2.11 | 2.00 | 3.75 | 4.89 | 4.04 | 4.19 | 4.15 | 4.04 | 4.25 | 2.71 | 2.29 | 4.00 |
| wall | | | | | | | | | | | | |
| | 0.5 | 0.0 | 1.60 | 0.31 | 0.91 | 1.0 | 0.53 | 0.81 | 0.59 | 0.90 | 1.01 | 1.26 |
| All | 4.36 | 3.94 | 4.75 | 4.86 | 3.81 | 4.60 | 4.69 | 4.57 | 4.61 | 4.22 | 2.92 | 4.22 |
| | 1.21 | 1.41 | 0.87 | 0.34 | 1.19 | 0.71 | 0.60 | 0.78 | 0.68 | 1.07 | 1.48 | 1.06 |

| | | | | |
|---|---|---|---|---|
| p Values | GC to AC: 0.00 | GC to CC: 0.00 | GC to CC: 0.13 | GC to CC: 0.00 |
| | GC to CC: 0.00 | GC to CC: 0.00 | GC to CC: 0.31 | GC to CC: 0.95 |
| | GC to CC: 0.00 | GC to CC: 0.00 | GC to CC: 0.60 | GC to CC: 0.00 |

Note:
Mean and SD values of acceptability score AS(X) for X ∈ {GC, AC, CC} are shown.
Abbreviations: AC, auto-contours; CC, clinical contour; GC, ground truth contour; OAR, organ at risk; SD, standard deviation.

Similarly, in Table 7, we list efficiency study results TC(X) for the two body regions. The total number of test samples available over all nine neck OARs that participated in this study was 462 (<9×60=540). This number for thorax over seven OARs was 433 (as some sites performed more than 60 studies). In the row labeled “Total,” we list the mean and SD values of total time taken per study estimated over all studies. In the next row, we list the p values of a t-test comparing CC to mAC for their total time taken per study and the percent saving in time by the HI system. For WashU, the numbers in the last three rows for neck could not be estimated as we did not have time estimates for all OARs in any study even when CtTr was excluded. This was not the case for thorax OARs.

TABLE 7
Efficiency Results

| | Penn | | NYPC | | WU | | RU | |
|---|---|---|---|---|---|---|---|---|
| OAR | CC | mAC | CC | mAC | CC | mAC | CC | mAC |

Neck: Total number of test samples for: CC-462, mAC-462

| | | | | | | | | |
|---|---|---|---|---|---|---|---|---|
| Cervical spinal cord | 2.24 | 0.96 | 3.42 | 1.35 | 2.42 | 1.61 | 1.09 | 0.43 |
| | 0.44 | 0.53 | 1.34 | 0.97 | 0.45 | 0.58 | 0.39 | 0.63 |
| Right parotid gland | 1.64 | 0.87 | 3.59 | 1.22 | 1.86 | 1.21 | 1.52 | 0.33 |
| | 0.37 | 0.50 | 0.74 | 1.59 | 0.34 | 0.78 | 0.52 | 0.76 |
| Left submandibular | 1.48 | 0.57 | 2.30 | 0.44 | 1.26 | 0.43 | 1.10 | 0.04 |
| gland | | | | | | | | |
| | 0.46 | 0.35 | 0.64 | 0.83 | 0.4 | 0.12 | 0.42 | 0.20 |
| Mandible | 5.66 | 0.79 | 5.34 | 1.00 | 7.23 | 1.89 | 4.23 | 0.20 |
| | 0.90 | 0.48 | 1.44 | 0.79 | 1.92 | 1.32 | 1.38 | 0.41 |
| Orohypopharyngeal | 1.88 | 0.98 | 3.66 | 1.49 | 3.65 | 1.08 | 4.04 | 1.45 |
| constrictor | | | | | | | | |
| muscles | | | | | | | | |
| | 0.35 | 0.46 | 0.55 | 1.16 | 0.33 | 0.25 | 0.92 | 1.12 |
| Cervical esophagus | 2.60 | 0.73 | 2.93 | 1.28 | 3.04 | 1.69 | 1.41 | 0.28 |
| | 0.63 | 0.54 | 0.69 | 1.32 | 0.84 | 0.65 | 0.89 | 0.65 |
| Supraglottic larynx | 2.23 | 1.85 | 3.25 | 3.11 | 1.51 | 0.70 | 1.10 | 0.52 |
| | 0.50 | 0.40 | 0.63 | 1.35 | 0.36 | 0.40 | 0.56 | 0.73 |
| Extended oral | 1.75 | 1.46 | 3.70 | 1.74 | 1.61 | 0.98 | 1.22 | 0.23 |
| cavity | | | | | | | | |
| | 0.33 | 0.46 | 0.94 | 1.60 | 0.29 | 0.44 | 0.59 | 0.43 |
| Cervical trachea | 1.05 | 0.14 | 1.81 | 0.65 | — | — | 1.16 | 0.50 |
| | 0.54 | 0.15 | 1.21 | 0.42 | | | 0.69 | 0.65 |
| Total | 20.67 | 8.48 | 29.55 | 11.57 | — | — | 16.39 | 4.00 |
| | 1.79 | 1.50 | 4.90 | 8.42 | | | 3.49 | 2.10 |
| p Value | 0.00 | | 0.00 | | — | | 0.00 | |
| Saving | 59% | | 61% | | | | 75% | |
| All sites | % Saving: | | | | | | | |
| | 66 | | | | | | | |

Thorax: Total number of test samples for: CC-433, mAC-433

| | | | | | | | | |
|---|---|---|---|---|---|---|---|---|
| Right lung | 1.26 | 0.37 | 1.57 | 1.05 | 4.46 | 2.47 | 3.32 | 1.18 |
| | 0.51 | 0.26 | 0.88 | 0.94 | 2.23 | 1.75 | 0.53 | 1.10 |
| Thoracic esophagus | 3.10 | 1.18 | 3.01 | 2.64 | 4.85 | 2.77 | 4.18 | 1.25 |
| | 0.66 | 0.95 | 0.84 | 1.92 | 1.73 | 2.28 | 1.00 | 1.24 |
| Extended proximal | 4.27 | 1.62 | — | — | 2.81 | 2.90 | 7.08 | 0.30 |
| bronchial tree | | | | | | | | |
| | 1.02 | 0.49 | | | 0.75 | 1.98 | 1.45 | 0.54 |
| Thoracic spinal | 1.59 | 0.05 | 2.26 | 0.71 | 3.80 | 1.13 | 2.81 | 0.30 |
| canal | | | | | | | | |
| | 0.36 | 0.11 | 0.87 | 0.47 | 0.87 | 0.67 | 0.60 | 0.75 |
| Heart | 2.13 | 1.19 | 3.42 | 1.14 | 4.90 | 4.94 | 4.95 | 1.87 |
| | 0.57 | 0.39 | 1.22 | 0.72 | 1.30 | 2.69 | 1.12 | 1.54 |
| Left chest wall | 6.56 | 0.68 | — | — | — | — | 8.40 | 0.93 |
| | 1.36 | 0.73 | | | | | 2.06 | 1.22 |
| Thoracic aorta | 2.80 | 0.81 | — | — | — | — | 4.60 | 1.30 |
| | 0.71 | 0.43 | | | | | 0.70 | 0.48 |
| Total | 21.70 | 5.88 | 9.73 | 5.51 | 15.69 | 10.31 | 30.40 | 6.00 |
| | 2.75 | 2.27 | 0.97 | 3.02 | 2.37 | 6.35 | 2.51 | 3.74 |
| p Value | 0.00 | | 0.01 | | 0.07 | | 0.00 | |
| Saving | 73% | | 42% | | 33% | | 80% | |
| All sites | % Saving: | | | | | | | |
| | 74 | | | | | | | |

Note:
Mean and SD values of time TC(X) for contouring for X ∈ {CC, mAC} are shown in minutes.
Abbreviations: CC, clinical contour; mAC, manually edited auto-contours; OAR, organ at risk; SD, standard deviation.

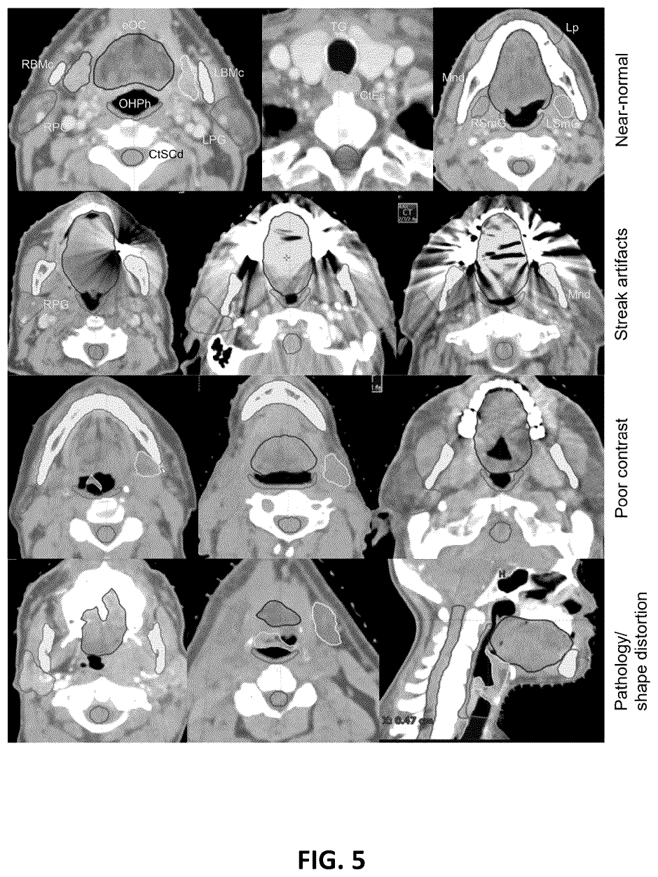

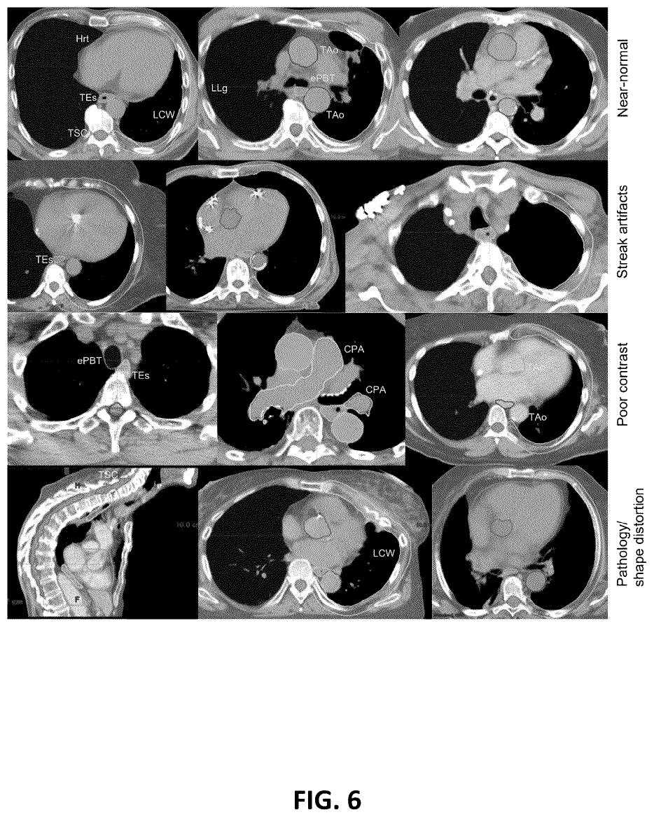

Cases with artifacts, pathology: In our cohort of clinical data sets, at least one of the issues related to image quality deviation occurs in 83% of the cases. The individual types of deviations and their frequencies are summarized in Table 8. To illustrate qualitatively the robustness of contouring by the HI system, we present a variety of cases in FIGS. 5 and 6 from neck and thorax, respectively, ranging from near-normal studies (first row in FIGS. 5 and 6) to cases with a severe degree of one or more of the deviations listed in Table 8. In these figures, the segmentation output by the HI system without mending (AC) is overlaid as an OAR boundary contour on CT image slices. Notably, even in their normal appearance, many OARs have poor contrast (third row in FIGS. 5 and 6) and are challenging to recognize and delineate. This issue is further exacerbated by the presence of streak artifacts (second row in FIGS. 5 and 6) and pathology-associated distortions (fourth row in FIGS. 5 and 6). Despite these deviations, the HI system robustly recognizes the OARs and delineates them very satisfactorily as is clear from the frequency listed in Table 8 and the results in Tables 5-7 in light of these deviations.

TABLE 8
Image quality deviations in our clinical data sets

| Image quality deviation | Neck (%) | Thorax (%) |
|---|---|---|
| Streak artifacts | 70 | 40 |
| Poor contrast | 30 | 30 |
| Shape distortion | 30 | 35 |
| Pathology | 70 | 70 |
| Implants | 60 | 30 |

TABLE 9
Computational time for training/model building and execution/testing
for the five different stages in the hybrid intelligence (HI) system

| | BRR | AAR | | DL-R | MM | DL-D | Total |
|---|---|---|---|---|---|---|---|
| Neck | | | | | | | |
| Training/model | ~1 day | 7 | min (39) | 10 days | — | 4.5 days | ~15.5 days |
| building | (400) | | | (125) | | (125) | |
| Execution (per | 47 s | 10 | min | 1 min 27 s | 1 min 13 s | 3 min 25 s | 17 min 2 s |
| study) | | | | | | | |
| Thorax | | | | | | | |
| Training/model | ~1 day | 7 | min (58) | 14 days | — | 3.8 days | ~18.8 days |
| building | (400) | | | (125) | | (125) | |
| Execution (per | 46 s | 11 | min | 1 min 30 s | 49 s | 2 min 22 s | 16 min 46 s |
| study) | | 19 | s | | | | |

Note:
The number of cases included in training for each module is listed in parenthesis.
Abbreviations: AAR, automatic anatomy recognition; BRR, body region recognition; DL-D, deep-learning-based object delineation; DL-R, deep-learning-based object recognition; MM, model morphing.

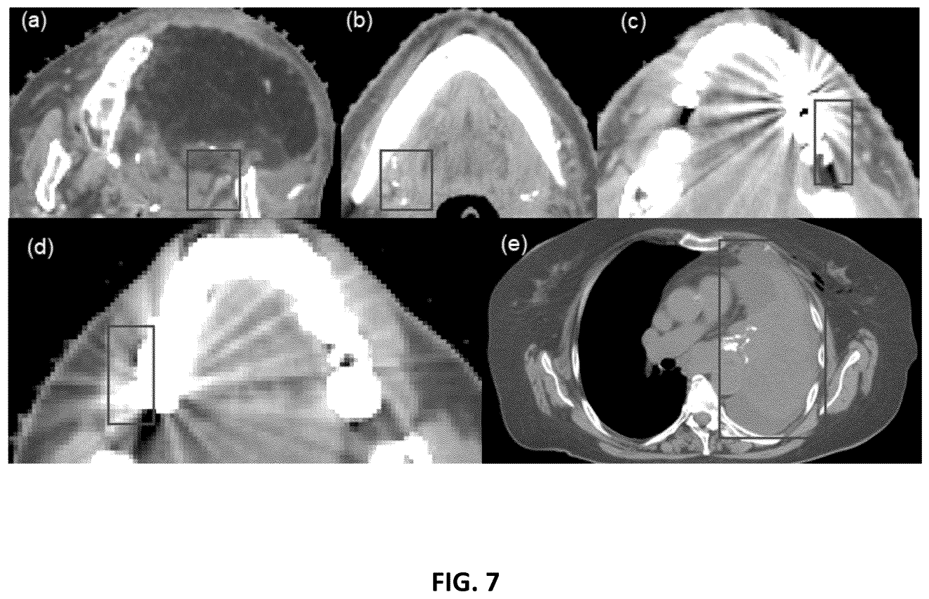

Cases of failure: Of the total number of OAR samples of 1760 in neck and 1040 in thorax, the HI system failed to segment 122 (6.93%) neck samples and 9 (0.86%) thorax samples. There were no cases where the image portrayed adequate information and yet the system failed to produce an output for an OAR sample. Most cases of “failure” occurred due to the OAR being completely removed or substantially obscured due to prior surgery, prior RT, or severe streak artifacts. A higher percentage of failure for necλ is understandable given the small size of OARs and their vulnerability to artifacts. The highest rate of “failure” was for the neck OARs: LSmG (16), right submandibular gland (RSmG) (24), left buccal mucosa (38), and right buccal mucosa (19). FIG. 7 displays some examples of failure cases for the two body regions.

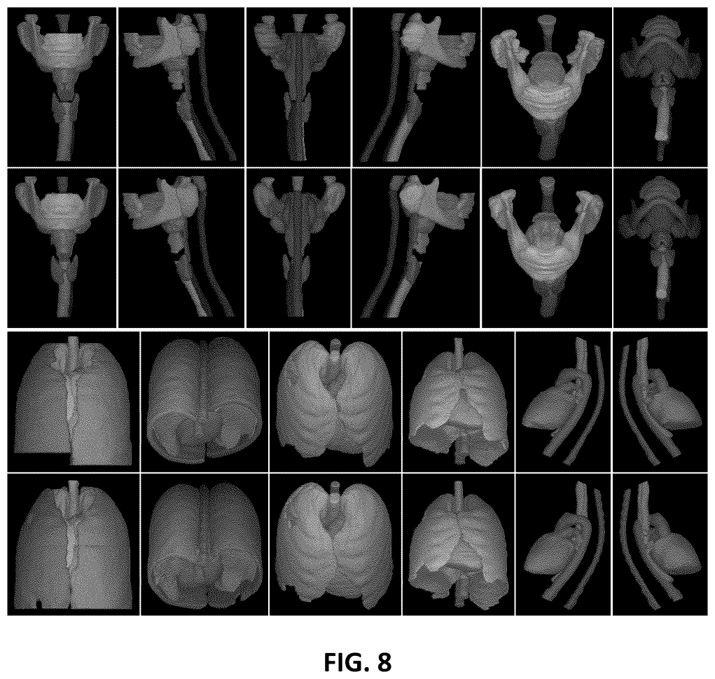

Finally, 3D renditions of the surfaces derived from GC and AC are displayed in FIG. 8 for cases randomly selected from among our clinical data sets. The renditions appear remarkably similar, revealing a behavior similar to what we already observed in Tables 5-7, but in a qualitative manner.

### Computational Considerations

Program execution times are estimated on a Dell computer with the following specifications: 6-core Intel i7-8700 3.2-GHz CPU with 32-GB RAM, RTX 2080ti with 11-GB RAM, and running the Linux Ubuntu operating system. The computational times of the training and execution processes for the different stages are listed in Table 11. Roughly, training time per body region is 15-18 days. (A purely DL-only approach without BRR, AAR-R, and MM, and with a vanilla form of BB detection for DL-R and the DL-D module described in this paper would take a total of ˜24 days for training.) Total execution time per body region is ˜17 min considering all operations. The current sequential implementation can be parallelized at the body region level, object-group (sparse/non-sparse) level, and object level to reduce training and execution times considerably. For example, DL-R and DL-D training can be conducted at the object-group and object level to reduce its time requirement by ˜10-fold. AARR recognizes objects based on order in the hierarchy. It is a serial process and hence takes a substantial portion (˜70%) of the total execution time in its current sequential implementation. AAR-R execution can be sped up by parallelizing implementation at the object level to run in about a minute, which can bring down the total execution time to ˜5 min per body region.

FIG. 5 shows neck cases. Examples ranging from near-normal studies to cases with a severe degree of one or more of the deviations listed in Table 12, with representative organs at risk (OARs). Row 4 demonstrates: Left: Axial contrast-enhanced computed tomography (CT) simulation image through the neck shows asymmetric thickening and sclerosis of the maxilla with associated distortion of the extended oral cavity. Middle: Axial non-contrast-enhanced CT simulation image through the neck demonstrates asymmetric fat stranding and loss of intervening fat planes in the right submandibular region and right posterior triangle as well as thickening of the right portion of the platysma muscle related to post-radiation changes with associated obscuration of the right submandibular gland. Right: Sagittal contrast-enhanced CT simulation image through the neck reveals a large mass in the tongue/floor of the mouth in the region of the extended oral cavity.

FIG. 6 shows thoracic cases. Examples ranging from near-normal studies to cases with a severe degree of one or more of the deviations listed in Table 8, with representative organs at risk (OARs). Row 4 demonstrates: left: Sagittal contrast-enhanced computed tomography (CT) simulation image through the thorax shows degenerative change and exaggerated kyphosis of the thoracic spine with associated distortion of the thoracic spinal canal and other intrathoracic structures. Middle: Axial non-contrast-enhanced CT simulation image through the thorax demonstrates postsurgical changes of prior partial rib cage resection and mesh repair in the anterior left chest wall with associated distortion of the shape of the left chest wall and left hemithorax. Right: Axial non-contrast-enhanced CT simulation image through the thorax reveals decreased volume of the right lung and mild rightward mediastinal shift related to prior partial right lung resection.

### 4|Discussion

### 4.1|Analysis of Results

From Tables 5-7, we make the following observations.

### 4.1.1|Accuracy

1. Over all neck OARs, accuracy of AC is better than that of CCs. The same is true for thorax OARs. The difference in accuracy is greater for neck OARs than for thorax OARs, understandably due to the sparser nature of the former and their greater susceptibility to streak artifacts, pathology, and posttreatment change.

2. AC compares favorably with mAC over all neck and thorax OARs. Among all neck OARs, although mAC is slightly better than AC for DC, AC shows a small advantage for HD. Thorax OARs also show a similar behavior, where AC has a small advantage for DC.

3. One of the reasons for CC to exhibit lower accuracy than AC is that RT centers have their own “contouring culture” although they all follow guidelines. This is the main reason behind our carefully establishing an independent GC, in addition to the requirement for the design of the HI system itself. Another reason is that CCs are drawn in a somewhat variable manner where the deviations from GC are deemed not important from the viewpoint of RT planning.

4. It is known that DC (to a lesser extent HD) behaves nonlinearly with respect to subjective acceptability.⁹⁸ That is, for the same level of acceptability, a small and sparse object like TEs exhibits a much lower DC than a larger well-defined non-sparse object like RLg. Most neck objects that resulted in a DC value of ˜0.7 for AC turned out to have excellent accuracy. Overall, the HI system performance exhibited excellent accuracy with respect to GC.

FIG. 7 is an illustration of cases of failure. The expected location of the organ at risk (OAR) is indicated by a region of interest (ROI). (a) Left submandibular gland (LSmG): Large postsurgical muscle flap containing fat in site of partial left mandibular resection which exerts mass effect upon other structures. (b) Right submandibular gland (RSmG): Low contrast and surgical clips/calcifications. (c) Left buccal mucosa (LBMc): Severe streak artifacts from dental implants in this region. (d) Right buccal mucosa (RBMc): Region of severe streak artifacts from dental implants. (e) Left lung (LLg): Lack of left lung aeration due to post-obstructive atelectasis caused by left perihilar tumor and left pleural effusion.

### 4.1.2 Acceptability

1. There is considerable inter-site variation in scores, which makes it difficult to draw conclusions over all sites. This also underscores “contouring culture” and its influence on scores. Overall, % of contours created by the HI system that scored ≥4 for the different institutions were as follows. Neck: Penn: 71.3%, NYPC: 89.3%, WashU: 72%, Rutgers: 86.6%. Thorax: Penn: 63.6%, NYPC: 63%, WashU: 89%.1, Rutgers: 36.7%. For neck OARs, Penn rated AC the best³, even better than GC; other sites rated GC the best, as to be expected, although the differences among GC, AC, and CC are meager. Some sites (Penn, WashU) seem to disagree with GC, again emphasizing differences in “contouring culture” and the need for standardized definitions.

2. The behavior is quite different for thorax OARs compared to neck OARs. For thorax OARs, NYPC and WashU rated contours in the order GC>CC>AC, as can be expected, whereas Penn rated CC>GC and Rutgers rated CC=GC, both placing AC lowest (lower scores are highlighted in Table 6). The behavior of Penn and Rutgers ensued mainly due to the influence from LCW and Hrt. For the LCW, for example, both sites considered GC inadequate. On slices, the LCW appears as a C-shaped region, including intercostal neurovascular bundles, ribs, chest wall muscles, and fat. This object is very challenging for auto-contouring and manual tracing due to the absence of intensity boundaries in the outward direction beyond the lung boundary. Both Penn and Rutgers seem to consider the width of LCW in GC to be inadequate.

3. Overall, there is an overwhelming consensus among sites on GC, indicating that standardly defined contours are more/consistently acceptable by all oncologists. Our results indicate that the HI system produced highly acceptable results consistent with the GC definitions adopted for the OARs.

### 4.1.3 Efficiency

1. For neck OARs, the savings in time from mAC over CC range from 59% to 75%. For thorax OARs, this range is 33%-80%. That is, the HI system can save 33%-80% of human operator time in routine RT clinical operations. Given the large fraction of the cases with serious deviations in image quality (Table 8) in our clinical cohort, this is remarkable.

2. All tested OARs demonstrated saved time. The savings are the greatest for LSmG, Mnd, TSC, LCW, and TAo. With the exception of SpGLx, eOC, and Hrt, all OARs achieve a time-savings of at least 50%.

3. Interestingly, although LCW was scored poorly for acceptability by Penn (2.0) and Rutgers (2.29), it achieved a time-saving of 89.4% and 85.1%, respectively, for the two sites. Similarly, TEs, which scored below 3 (by Rutgers with AS=2.75) showed a time-saving of ˜-70%.

FIG. 8 shows 3D surface renditions in different combinations of neck (rows 1, 2) and thorax organs at risk (OARs) (rows 3, 4) from ground truth contours (rows 1, 3) and auto-contours (rows 2, 4)

The previous findings and the known nonlinear behavior of accuracy metrics⁹⁸ suggest the importance of analyzing the three groups of metrics jointly to develop a solid understanding of the behavior of any auto-contouring system. DC to AS relationship is known to be nonlinear. As illustrated previously, the relationships between AS and efficiency metric TC as well as between DC and TC also demonstrate nonlinearity. Without delving into outcome considerations of the RT application and focusing on technical algorithmic evaluation, efficiency seems to be an effective arbiter of performance-how much human time is needed in editing the AC to make them agreeable to human experts. Obviously, the currently ubiquitous accuracy measures DC and HD alone are extremely inadequate.

4.2 Comparison with Methods from the Literature

A direct comparison of existing methods on segmentation challenge data sets with the HI system is infeasible as the data sets used, acquisition protocols and resolutions, considered objects, scanner brand variability, image deviations due to abnormalities, and most importantly the GT definitions of the objects are nonexistent or not specified in these methods/data sets. The last point renders it impossible to make a fair comparison of the HI system with these methods/existing challenge data sets without compromising the very tenet of the HI system for the following reasons: (RI) None of these methods/data sets provide a definition of the body region or objects contained in them. Recall how this is most crucial (Section 2.2) for the HI system for encoding NI. A consequence of this lapse is that OARs which cross body regions will have undefined superior and inferior extent that can severely affect the accuracy of the HI system. Similar comments are applicable to all other objects in the body region. A possible solution is for us to recreate GT for these public data sets following the tenets of our HI-methodology and assess performance. But then, the new GT will not be relevant for existing methods that reported results previously based on GT provided by the challenge. (R2) None of the published methods^{47,74,75,77-80,86,90} have shown performance on data sets coming from such varied brands of scanners (Table 3) and distribution of image deviations (Table 8) as demonstrated in this paper. (R3) None of them performed evaluation utilizing all three classes of metrics-accuracy, acceptability, and efficiency.

To demonstrate R1, we conducted two studies as follows.

Study 1: We selected two public datasets relevant for our application: 2015 MICCAI Head and Neck Auto Segmentation Challenge data set⁹⁹ and 2019 SegTHOR challenge data set.¹⁰⁰ We focused on the recognition problem rather than delineation to determine how R1 affects object localization directly and due to the fact that poor recognition leads to poor delineation. We compared recognition accuracy we achieved on our data sets with the accuracy obtained on the two challenge data sets. We focused on those OARs in these challenge data sets that we have considered in our HI system. For the reasons already mentioned under RI and for generalizability consideration, we did not retrain our models on the challenge data sets. Accuracy metrics used are as follows: (i) LE: centroid location error expressed as the distance between true centroid location and the centroid of the fuzzy model FM_{O}(I_{B}) output by the final recognition module MM (FIG. 1), ideal value being 0; (ii) SE: size error defined as the size of the detected object (fuzzy model) divided by the true size of the object (see Ref. [30] for details on how size is defined via principal component analysis), ideal value being 1. The results are summarized in Table 12.

TABLE 10
Error in recognition, location error (LE) (in mm) and size error (SE), for neck and thorax
challenge data sets, shown under “Challenge” and our data sets shown under column “Our”
Neck

| | CtBrStm | | Mnd | | LPG | | RPG | | LSmG | | RSmG | |
|---|---|---|---|---|---|---|---|---|---|---|---|---|
| | Challenge | Our | Challenge | Our | Challenge | Our | Challenge | Our | Challenge | Our | Challenge | Our |
| LE (mm) | 5.5 | 2.0 | 10.7 | 5.4 | 4.5 | 3.4 | 4.9 | 2.9 | 6.8 | 3.7 | 4.6 | 3.5 |
| | 3.4 | 1.0 | 3.0 | 5.7 | 2.2 | 3.0 | 2.1 | 2.6 | 4.2 | 5.7 | 2.8 | 2.4 |
| SE | 0.98 | 1.09 | 0.91 | 0.92 | 0.87 | 1.06 | 0.78 | 1.12 | 0.87 | 0.91 | 1.12 | 0.92 |
| | 0.03 | 0.10 | 0.18 | 0.03 | 0.10 | 0.11 | 0.11 | 0.11 | 0.09 | 0.19 | 0.05 | 0.12 |

Thorax

| | | | |
|---|---|---|---|
| Hrt | TEs | ePBT | Tao |

| | Challenge | Our | Challenge | Our | Challenge | Our | Challenge | Our |
|---|---|---|---|---|---|---|---|---|
| LE (mm) | 6.8 | 6.8 | 6.9 | 4.7 | 8.5 | 6.8 | 10.8 | 3.8 |
| | 6.1 | 8.7 | 4.2 | 3.4 | 5.6 | 4.7 | 7.9 | 4.4 |
| SE | 0.99 | 1.02 | 0.98 | 0.99 | 1.17 | 0.99 | 1.55 | 0.94 |
| | 0.04 | 0.03 | 0.07 | 0.07 | 0.13 | 0.06 | 0.10 | 0.02 |

Note:
Mean (first entry) and standard deviation (second entry) values over the tested samples are listed.
Abbreviations: CtBrStm, cervical brainstem; ePBT, extended proximal bronchial tree; Hrt, Heart; LPG, Left parotid gland; LSmG, left submandibular gland; Mnd, Mandible; RPG, Right parotid gland; RSmG, right submandibular gland; Tao, thoracic aorta; TEs, Thoracic esophagus.

We note that for both LE and SE, accuracy deteriorates on challenge data sets, substantially for objects that cross the body region, namely, cervical brainstem (CtBrStm), Mnd, TEs, ePBT, and TAo. Other objects also seem to be affected for reasons explained earlier.

Study 2: We selected 2 OARs—CtBrStm and TEs—from the previous neck-and-thorax challenge data sets and obtained final delineations S_{O}(I). As mentioned before, these objects cross the respective body region boundaries. Table 13 summarizes the resulting delineation accuracy metrics for these OARs. Notably, accuracy deteriorates compared to the results from the HI system (reproduced from Table 5 under column labeled “Our”). The mean DC and mean HD both decrease for both objects.

TABLE 11
Delineation accuracy (mean, standard deviation [SD]) for
cervical brainstem (CtBrStm) and thoracic esophagus (TEs)
on challenge data sets and our data sets

| | DC | | HD | |
|---|---|---|---|---|
| | Challenge | Our | Challenge | Our |
| CtBrStm | 0.61 | 0.74 | 4.76 | 2.91 |
| | 0.06 | 0.10 | 1.27 | 3.07 |
| TEs | 0.76 | 0.79 | 7.03 | 3.55 |
| | 0.10 | 0.09 | 8.21 | 4.58 |

Abbreviations:
CtBrStm, cervical brainstem;
DC, Dice coefficient;
HD, Hausdorff distance.

Publications reporting works that are somewhat related to our work in spirit are Refs. [47, 74, 75, 77-80, 86, 90]. In Table 12, we present a comparison of these methods to our HI system based on the results reported in these works. Among these methods, Refs. [47, 74, 75, 77-79] focus only on the neck, and Refs. [80, 86] only on the thorax. Although Ref. [90] dealt with three different body regions, including head, thorax, and pelvis, only one object was tested in each body region. One study⁴⁷ performed efficiency assessment of how the method reduces delineation time compared with fully manual contouring, although only on a small test set of 10 cases where nothing is mentioned about the composition of these data sets with regard to image quality, images from different institutions, images from different scanners, and so on. Beyond that, none of the methods have assessed efficiency and acceptability and dealt with challenging cases as in our evaluation. For the accuracy aspect, ignoring other issues, for the same objects (LSmG, RSmG, CtEs, SpGLx, and Hrt), our results are comparable to, and often better than, the current results from the literature, especially considering the large number of extremely challenging cases among our data sets. The results reported in Ref. [86] are not based on a fully automatic approach, but a method where the BBs were all manually specified.

TABLE 12
Comparison with current methods from the literature that are related to our work

| | | | Dataset/training- | Voxel | | | | |
|---|---|---|---|---|---|---|---|---|
| | | | to test data | size | | | | Difficult |
| Method | Objects | Image | proportion | (mm³) | Accuracy | Efficiency | Acceptability | cases |
| Wang | 9 Neck | CT | MICCAI 2015 | (0.76- | DC: 45.1%- | ~ | ~ | ~ |
| et al.⁷⁴ | organs | | Challenge data | 1.25)² × | 93% | | | |
| | | | set/33-15 | 1.25- | 95HD: | | | |
| | | | | 3 | 1.26-3.44 | | | |
| | | | | | mm | | | |
| Men et | 7 Neck | CT | 80-20, fivefold | (0.88- | DC (mean): | ~ | ~ | ~ |
| al.⁷⁵ | organs | | | 1.27)² × | 90% | | | |
| | | | | 1.25- | (86%- | | | |
| | | | | 3 | 93%) | | | |
| Guo et | 42 Neck | CT | 142 in total, | 0.95² × | DC (mean): | ~ | ~ | ~ |
| al.⁷⁷ | organs | | multiple | 1.9 | 75. % | | | |
| | | | fourfold | (mean) | HD (mean): | | | |
| | | | | | 7 mm | | | |
| | 9 Neck | CT | MICCAI 2015 | (0.76- | DC (mean): | | | |
| | Organs | | Challenge data | 1.25)² × | 82.4% | | | |
| | | | set/33-15 | 1.25- | | | | |
| | | | | 3 | | | | |
| Guo et | 18 Neck | CT | 40-10 | ~ | DC (mean) | | | |
| al.⁷⁸ | Organs | | | | 72.5% | | | |
| | 9 Neck | CT | MICCAI 2015 | (0.76- | DC (mean) | | | |
| | Organs | | Challenge data | 1.25)² × | 80.3% | | | |
| | | | set/33-15 | 1.25- | 95HD | | | |
| | | | | 3 | (mean): 2.62 | | | |
| | | | | | mm | | | |
| Liang | 11 Neck | CT | 23-73 | (0.36- | DC (mean): | | | |
| et al.⁷⁹ | Organs | | | 0.69)² × | 80.3% | | | |
| | | | | 3 | (86.8%- | | | |
| | | | | | 93%) | | | |
| | 9 Neck | CT | MICCAI 2015 | (0.76- | DC: 71.3%- | | | |
| | Organs | | Challenge data | 1.25)² × | 94.1% | | | |
| | | | set/33-15 | 1.25- | | | | |
| | | | | 3 | | | | |
| Liao et | Brain | MRI | BraTS2015/234- | ~ | DC (mean): | ~ | ~ | ~ |
| al.⁹⁰ | tumor | | 40 | | 88.5% | | | |
| | Heart | MRI | MM-WHS/16-4 | ~ | DC (mean): | | | |
| | | | | | 86.9% | | | |
| | Prostate | MRI | NCI- | ~ | DC (mean): | | | |
| | | | ISBI2013/64- | | 82.7% | | | |
| | | | 16 | | | | | |
| Tang et | 28 Neck | CT | Combined 2 | (0.98)² × | DC (mean): | Reports | ~ | ~ |
| al.⁴⁷ | organs | | different data | 2-3 | 78.3% | 61% | | |
| | | | sets | | 95HD | reduction | | |
| | | | together/215- | | (mean): | in | | |
| | | | 100 | | 6.21, 6.28 | time on | | |
| | | | | | on two test | 10 cases | | |
| | | | | | sets, | | | |
| | | | | | respectively | | | |
| | 9 Neck | CT | MICCAI 2015 | (0.76- | DC (mean): | | | |
| | organs | | Challenge data | 1.25)² × | 81.2%, | | | |
| | | | set/33-15 | 1.25- | 72.3%- | | | |
| | | | | 3 | 95% | | | |
| El | Heart, | CT | SegTHOR/40- | (0.9- | DC: 91.6%- | ~ | ~ | ~ |
| Jurdi et | aorta, | | 20 | 1.37)² × | 98.3% | | | |
| al.⁸⁶ | trachea, | | | 2-3.7 | (mean): | | | |
| | esophagus | MRI | 14-6 | ~ | 89.9% | | | |
| | Heart | | | | HD (mean): | | | |
| | | | | | 2.17 mm | | | |
| Trullo | Heart, | CT | 25-5, sixfold | (0.98)² × | DC: 69%- | ~ | ~ | ~ |
| et al.⁸⁰ | aorta, | | | 2.5 | 89% | | | |
| | trachea, | | | | | | | |
| | esophagus | | | | | | | |
| HI | 16 Neck | CT | 125-110 | (0.98- | DC (mean): | Mean of | AS: 3.83- | 85% of |
| system | organs | | | 1.27)² × | 78%, | 66% | 4.45 (5 is the | the test |
| | | | | 1.5-3 | 61%-92% | (59%- | highest) | cases |
| | | | | | HD (mean): | 75%) | from four | has |
| | | | | | 2.22, | reduction | sites on 142 | image |
| | | | | | 1.22-3.09 | in | test samples | quality |
| | | | | | mm | time on | | deviation |
| | | | | | | 462 test | | issues |
| | | | | | | samples | | |
| | 10 | CT | 125-104 | (0.98- | DC (mean): | Mean of | AS: 2.92- | 80% of |
| | Thorax | | | 1.60)²× | 87%, | 74% | 4.57 (5 | the test |
| | | | | | | (33%- | is the | cases |
| | | | | | | 80%) | highest) | has |
| | | | | | | reduction | from four | image |
| | | | | | | in | sites | quality |
| | | | | | | time on | on 150 test | deviation |
| | | | | | | 433 | samples | |
| | | | | | | test | | |
| | | | | | | samples | | |
| | Brainstem | CT | MICCAI 2015 | (0.76- | DC (mean): | ~ | ~ | ~ |
| | | | Challenge data | 1.25)² × | 61%, | | | |
| | | | set | 1.25-3 | 53.4%- | | | |
| | | | | | 74.3% | | | |
| | | | | | HD (mean): | | | |
| | | | | | 3.9, | | | |
| | | | | | 2.59-5.64 | | | |
| | | | | | mm | | | |
| | Esophagus | CT | SegTHOR | (0.9- | DC (mean): | ~ | ~ | ~ |
| | | | | 1.37)² × | 76%, | | | |
| | | | | 2-3.7 | 59.6%- | | | |
| | | | | | 88.9% | | | |
| | | | | | HD (mean): | | | |
| | | | | | 7.03, | | | |
| | | | | | 0.95-29.08 | | | |
| | | | | | mm | | | |

Note:
Unknown and irrelevant entries are indicated by “~”.
Abbreviations: CT, computed tomography; DC, Dice coefficient; HD, Hausdorff distance; HI, hybrid intelligence.

In summary, no methods in the literature have performed as thorough an evaluation as in the current study and demonstrated behavior on real-life cases with severe artifacts, poor contrast, shape distortion (including from post-treatment change), pathology, and implants. Furthermore, due to the lack of any definitions for body regions and OARs in available public challenge data sets, it becomes impossible to perform a fair comparison of our HI system to existing methods, as we demonstrated, as the very definition itself is one of the founding principles of our system.

### 5|CONCLUSIONS

In this paper, we described a novel HI-methodology to combine anatomic knowledge coming from NI with advanced DL techniques (AI) under a novel recognition-delineation paradigm to accomplish organ segmentation in a highly robust manner. In the processing pipeline, NI is infused at various stages of the HI system, including in the design of the network architecture. We demonstrated the performance of the HI system on an entirely independent cohort of real-life clinical data sets obtained at four different clinical RT centers utilizing different brands of CT scanners under various image acquisition conditions. The clinical data sets covered a wide range of deviations from normal anatomy and image appearance due to streak artifacts, poor contrast, shape distortion (including from posttreatment change), pathology, and implants. In our experience to date, the system behaves almost like an expert human but vastly more efficiently in the contouring task for RT planning.

Among the two body regions tested, neck OARs are considerably more challenging where our system shows its full power, and the time-savings are commensurately better for neck than for thorax. As illustrated in FIGS. 5 and 6, the system uses NI help where image information alone will not suffice to decide, first for the correct localization (recognition) of the object and then for the precise delineation of the boundary.

Assessment of accuracy, acceptability, and efficiency sheds light on the total behavior of the HI system as well as the limitations in currently used metrics. Accuracy as an initial technical bench test measure is fine but is inadequate to express the clinical suitability of the contours. Acceptability tests are expensive to conduct and do not reflect the ease of post hoc mendability of contours. Efficiency seems to be the single most useful metric, but it is also expensive to assess. In our current assessment, no post-processing of the output of the HI system was performed, for example, to remove isolated debris, and so on. This would have improved DC and HD for some objects such as Hrt. We are investigating smart methods to perform post hoc correction of contours and automatic assessment of efficiency.

There are two main current limitations of the HI-approach. (i) Amorphous objects that cannot be modeled adequately (such as left and right thoracic brachial plexuses) remain challenging to auto-contour. We are investigating methods of advanced shape modeling to handle such objects. (ii) Auto-contouring execution time. Currently, in its sequential and non-optimized implementation of some of the modules, the HI system requires 20-30 min to segment all OARs in a body region, including some housekeeping operations. Our goal is to bring this down to ˜5 min. We are investigating parallelization strategies to implement some of these steps.

### Endnotes

1 Medical image segmentation is uniquely different from general image segmentation problems in computer vision in that in the former, we have rich modellable prior knowledge. This is chiefly the facilitator for HI approaches.

2 A fundamental question related to the RD-paradigm is as follows: What does the act of human recognition of an object actually mean and how to translate that concept to a computable paradigm? There seem to be two pieces of information associated with this concept: object location and object extent. We argue that the R-step of the AAR methodology³⁰ and the fuzzy model output by it constitute something that comes remarkably close to such a paradigm. The geometric center of the fuzzy model indicates rough (fuzzy) location and the fuzzy membership in the model suggests rough (fuzzy) extent of occupation. This indeed was the rationale and motivation for the original design of AAR³⁰ and its RD-paradigm.

3 We note that the clinicians from Penn Radiation oncology who rated acceptability were completely blinded to the detailed object and body region definitions employed in our system. They were not part of the team that developed the HI-system.

4 Even axial (transverse) planes may not be fully adequate in completely defining certain body region boundaries. For example, in separating the thoracic body region from the abdominal body region, the surfaces of the left and right hemi-diaphragmatic domes may also have to be considered, where the space superior to these surfaces belongs to the thoracic body region and the space inferiorly forms the abdominal body region.

5 Unfortunately, the numerous publicly available data sets under “segmentation challenges” have not considered this issue of precise body region/object definition. As such, their utility is diminished. More seriously, the HI-systems like the one proposed here cannot perform fair comparative assessment using those data sets without sacrificing their own performance.

6 This activity was directed by a physician-scientist-radiologist (Torigian) with ˜25 years of experience in multiple modalities/body regions/subspecialties) assisted by a computational imaging researcher (Udupa) with ˜45 years of experience in multiple modalities/body regions/imaging applications. This NI-AI combination, even in guidance and training was crucial to ensure that appropriate medical knowledge is properly implemented in the GT creation process.

### REFERENCES

1. Doyle W. Operations useful for similarity-invariant pattern recognition. JACM 1962; 9:259-267.
2. Narasimhan R, Fornango J P. Some further experiments in the parallel processing of pictures. IEEE Trans Electron Comput. 1963; EC-13:748-750.
3. Liu H. Two- and three-dimensional boundary detection. Comput Graph Image Process. 1977; 6:123-134.
4. Herman G T, Srihari S, Udupa J K. Detection of changing boundaries in two- and three-dimensions. In: Badler N I, Aggarwal J K, eds. Proceedings of the Workshop on Time Varying Imagery. University of Pennsylvania; 1979:14-16.
5. Pope D, Parker D, Gustafson D, Clayton P. Dynamic search algorithm in left ventricular border recognition and analysis of coronary arteries. IEEE Proc Comput Cardiol. 1984; 9:71-75.
6. Kass M, Witkin A, Terzopoulos D. Snakes: active contour models. Int J Comput Vision. 1987; 1:321-331.
7. Mumford D, Shah J. Optimal approximations by piecewise smooth functions and associated variational problems. Commun Pure Appl Math. 1989; 42:577-685.
8. Wu Z, Leahy R. An optimal graph-theoretic approach to data clustering: theory and its application to image segmentation. IEEE Trans Pattern Anal Mach Intell. 1993; 15(11):1101-1113.
9. Beucher S. The watershed transformation applied to image segmentation. 10th Pfefferkorn Conference on Signal and Image Processing in Microscopy and Microanalysis. 1992:299-314.
10. Bezdek J, Hall L, Clarke L. Review of MR image segmentation techniques using pattern recognition. Med Phys. 1993; 20:1033-1048.
11. Malladi R, Sethian J, Vemuri B. Shape modeling with front propagation: a level set approach. IEEE Trans Pattern Anal Mach Intell. 1995; 17:158-175.
12. Udupa J K, Samarasekera S. Fuzzy connectedness and object definition: theory, algorithms, and applications in image segmentation. Graphical Models Image Process. 1996; 58:246-261. https://doi.org/10.1006/gmip.1996.0021
13. Falcao A, Udupa J K, Samarasekera S, Sharma S, Hirsch B E, Lotufo R. User-steered image segmentation paradigms: live wire and live lane. Graphical Models Image Process. 1998; 60:233-260. https://doi.org/10.1006/gmip.1998.0475
14. Xu C, Prince J L. Snakes, shapes, and gradient vector flow. IEEE Trans Image Process. 1998; 7(3):359-369.
15. Boykov Y, Veksler O, Zabih R. Fast approximate energy minimization via graph cuts. IEEE Trans Pattern Anal Mach Intell. 2001; 23:1222-1239.
16. Pham D L, Xu C, Prince J L. Current methods in medical image segmentation. Annu Rev Biomed Eng. 2000; 2:315-337.
17. Ungrua K, Jiang X. Dynamic programming-based segmentation in biomedical imaging. Comput Struct Biotechnol J. 2017; 15:255-264.
18. Summa B, Faraj N, Licorish C, Pascucci V. Flexible livewire: image segmentation with floating anchor. Eurographics. 2018; 37(2):321-328.
19. Baxter J S H, Gibson E, Eagleson R, Peters T_{M}. The semiotics of medical image segmentation. Med Image Anal. 2017; 44:54-η.
20. Farag T H, Hassan W A, Ayad H, AI Bahussain A S, Badawi U A, Alsmadi M K. Extended absolute fuzzy connectedness segmentation algorithm utilizing region and boundary-based information. Arabian J Sci Eng. 2017; 42:3573-3583. https://doi.org/10.1007/si3369-017-2577-0. Published online May 2017.
21. Lamboi U, Fofana I, Kamara Y L. Superiority of graph-based visual saliency (GVS) over other image segmentation methods. Int J Sci Technol Res. 2017; 6(2):14-21.
22. Staib L H, Duncan J S. Boundary finding with parametrically deformable models. IEEE Trans Pattern Anal Mach Intell. 1992; 14:1061-1075.
23. Cootes T, Taylor C, Cooper D. Active shape models: their training and application. Comput Vision Image Understanding. 1995; 61:38-59.
24. Park J, Metaxas D N, Axel L. Analysis of left ventricular wall motion based on volumetric deformable models and MRISPAMM. Med Image Anal. 1996; 1(1):53-71.
25. Pizer S M, Fletcher P T, Joshi S, et al. Deformable M-reps for 3D medical image segmentation. Int J Comput Vision. 2003; 55:85-106.
26. Tao X, Prince J L, Davatzikos C. Using a statistical shape model to extract sulcal curves on the outer cortex of the human brain. IEEE Trans Med Imaging. 2002; 21:513-524.
27. Liu J M, Udupa J K. Oriented active shape models. IEEE Trans Med Imaging. 2009; 28:571-584.
28. Metaxas D N. Physics-based deformable models: applications to computer vision. In: Graphics and Medical Imaging. Springer Science & Business Media; 2012.
29. Shen T A, Li H S, Huang X L. Active volume models for medical image segmentation. IEEE Trans Med Imaging. 2011; 30:774-791.
30. Udupa J K, Odhner D, Zao L, et al. Body-wide hierarchical fuzzy modeling, recognition, and delineation of anatomy in medical images. Med Image Anal. 2014; 18:752-771. https://doi.org/10.1016/j.media.2014.04.003
31. Niethammer M, Pohl K M, Janoos F, Wells W M. Active mean fields for probabilistic image segmentation: connections with Chan-Vese and Rudin-Osher-Fatemimodels. SIAM J Image Sci. 2015; 10(3):1069-1103.
32. Gee J, Reivich M, Bajcsy R. Elastically deforming 3D atlas to match anatomical brain images. J Comput Assist Tomogr. 1993; 17:225-236.
33. Christensen G, Rabbitt R, Miller M. 3-D brain mapping using a deformable neuroanatomy. Phys Med Biol. 1994; 39:609-618.
34. Shattuck D W, Mirza M, Adisetiyo V, et al. Construction of a 3D probabilistic atlas of human cortical structures. Neuroimage. 2008; 39:1064-1080.
35. Pohl K M, F isher J, Bouix S, Shenton M, McCarley R W, Grimson W E L. Using the logarithm of odds to define a vector space on probabilistic atlases. Med Image Anal. 2007; 11(5):465-477.
36. Ashbumer J, Friston K J. Computing average shaped tissue probability templates. Neuroimage. 2009; 45:333-341.
37. Landman B A, Warfield S K. MICCAI2012 Workshop on Multi-Atlas Labeling, 2. CreateSpace Independent Publishing Platform; 2012.
38. Linguraru M G, Pura JA, Pamulapati V, Summers R M. Statistical 4D graphs for multi-organ abdominal segmentation from multiphase CT. Med Image Anal. 2012; 16:904-914.
39. Asman A J, Landman B A. Non-local statistical label fusion for multi-atlas segmentation. Med Image Anal. 2013; 17:194-208.
40. Shi C, Cheng Y, Wang J, Wang Y, Mori K, Tamura S. Low-rank and sparse decomposition based shape model and probabilistic atlas for automatic pathological organ segmentation. Med Image Anal. 2017; 38:30-49.
41. Huo Y, Xu Z, Xiong Y, et al. 3D whole brain segmentation using spatially localized atlas network tiles. Neuroimage. 2019; 194:105-119.
42. Yan Z, Zhan Y, Peng Z, et al. Multi-instance deep learning: discover discriminative local anatomies for body part recognition. IEEE Trans MedImaging. 2016; 35(5):1332-1343.
43. Moeskops P, Viergever M A, Mendrik A M, de Vries L S, Benders JNLM, Ivana Isgum I. Automatic segmentation of brain MR images with a convolutional neural network. IEEE Trans Med Imaging. 2016; 35(5):1252-1262.
44. Drozdzal M, Chartrand G, Vorontsov E, et al. Learning normalized inputs for iterative estimation in medical image segmentation. Med Image Anal. 2018; 44:1-13.
45. Mortazi A, Bagci U. Automatically designing CNN architectures for medical image segmentation. In: Shi Y, Suk H I, Liu M (eds). Machine Learning in Medical Imaging. MLMI 2018. Lecture Notes in Computer Science (Machine Learning in Medical Imaging-MICCAI).vol. 11046.Springer,Cham.https://doi.org/10.1007/978-3-030-00919-9_12
46. Lustberg T, van Soest J, Gooding M, et al. Clinical evaluation of atlas and deep learning based automatic contouring for lung cancer. Radiother Oncol. 2018; 126:312-317.
47. Tang H, Chen X, Liu Y, et al. Clinically applicable deep learning framework for organs at risk delineation in CT images. Nat Mach Intell. 2019; 1:480-491. https://doi.org/10.1038/s42256-019-0099-z
48. Zhou S K, Greenspan H, Davatzikos C, et al. A review of deep learning in medical imaging: imaging traits, technology trends, case studies with progress highlights, and future promises. Proceedings of the IEEE. 2021; 109(5):820-838. https://doi.org/10.1109/JPROC.2021.3054390
49. Weisman A J, Kieler M W, Perlman S B, et al. Convolutional neural networks for automated PET/CT detection of diseased lymph node burden in patients with lymphoma. Radiol: Artif Intell. 2020; 2:e200016. https://doi.org/10.1148/ryai.2020200016
50. Renard F,Guedria S, Noel De Palma N D, Vuillerme N. Variability and reproducibility in deep learning for medical image segmentation. Nat Res. 2020; 10:13724. https://doi.org/10.1038/s41598-020-69920-0
51. Han S, Prince J L, Carass A. Reflection-equivariant convolutional neural networks improve segmentation over reflection augmentation. Proceedings of SPIE, Medical Imaging: Image Processing. 2020; 11313:1131337, https://doi.org/10.1117/12.2549399
52. Wu X, Udupa J K, Tong Y, et al. AAR-RT—a system for auto-contouring organs at risk on CT images for radiation therapy planning: principles, design, and large-scale evaluation on head-and-neck and thoracic cancer cases. Med Image Anal. 2019; 54:45-62. https://doi.org/10.1016/j.media.2019.01.008
53. Wang H, Udupa J K, Odhner D, Tong Y, Zhao L, Torigian D A. Automatic anatomy recognition in whole-body PET/CT images. Med Phys. 2016; 43(1):613-629.
54. Sun K, Udupa J K, Odhner D, Tong Y, Zhao L, Torigian D A. Automatic thoracic anatomy segmentation on CT images using hierarchical fuzzy models and registration. Med Phys. 2016; 43(3):1487-1500. https://doi.org/10.1118/1.4942486
55. Xu G, Udupa J K, Tong Y, Odhner D, Cao H, Torigian D A. AARLN-DQ: automatic anatomy recognition based disease quantification in thoracic lymph node zones via FDG PET/CT images without nodal delineation. Med Phys. 2020; 47(8):3467-3484. https://doi.org/10.1002/mp.14240
56. Matsumoto M S M, Udupa J K, Saboury B, Torigian D A. Quantitative normal thoracic anatomy at CT. Comput Med Imaging Graph. 2016; 51:1-10. https://doi.org/10.1016/j.compmedimag.2016.03.005
57. Kelm B M, Wels M, Zhou S K, et al. Spine detection in CT and MR using iterated marginal space learning. Med Image Anal. 2013; 17(8):1283-1292.
58. Zhou X, Wang S, Chen H, et al. Automatic localization of solid organs on 3D CT images by a collaborative majority voting decision based on ensemble learning. Comput Med Imaging Graph. 2012; 36:304-313.
59. Criminisi A, Robertson D, Konukoglu E, et al. Regression forests for efficient anatomy detection and localization in computed tomography scans. Med Image Anal. 2013; 17:1293-1303.
60. Seifert S, Barbu A, Zhou K S, et al. Hierarchical parsing and semantic navigation of full body CT data. Proc SPIE Med Imaging Conf. 2009; 7259:725902-1-725902-8.
61. Mamani G E H, Setio A A A, van Ginneken B, Jacobs C. Organ detection in thorax abdomen CT using multi-label convolutional neural networks. Proc SPIE, Med Imaging: Comput-Aided Diagn. 2017; 10134:1013416.
62. Humpire-Mamani G E, Setio A A A, van Ginneken B, Jacobs C. Efficient organ localization using multi-label convolutional neural networks in thorax-abdomen CT scans. Phys Med Biol. 2018; 63(8):085003.
63. Linder T, Jigin O. Organ Detection and Localization in Radiological Image Volumes. Master's thesis. Department of Computer Science, Linköping University; 2017.
64. Yu Q, Xie L, Wang Y, Zhou Y, F ishman EK, Yuille A L. Recurrent saliency transformation network: incorporating multi-stage visual cues for small organ segmentation. Proceedings of the IEEE Conference on Computer Vision and Pattern Recognition. 2018:8280-8289.
65. Roth H R, Lu L, Lay N, et al. Spatial aggregation of holistically-nested convolutional neural networks for automated pancreas localization and segmentation. Med Image Anal. 2018; 45:94-107. https://doi.org/10.1016/j.media.2018.01.006
66. Zhou X, Kojima T, Wang S, et al. Automatic anatomy partitioning of the torso region on CT images by using a deep convolutional network with majority voting. Proc SPIE, Med Imaging: Comput-Aided Diagn. 2019; 10950:109500Z.
67. Man Y, Huang Y, Feng J, Li X, Wu F. Deep Q learning driven CT pancreas segmentation with geometry-aware U-Net. IEEE Trans Med Imaging. 2019; 38(8):1971-1980. https://doi.org/10.1109/TMI.2019.2911588
68. Xu X, Zhou F, Liu B, Fu D, Bai X. Efficient multiple organ localization in CT image using 3D region proposal network. IEEE Trans Med Imaging. 2019; 38(8):1885-1898. https://doi.org/10.1109/TMI.2019.2894854
69. Liu C, Hu S C, Wang C, Lafata K, Yin F F. Automatic detection of pulmonary nodules on CT images with YOLOv3: development and evaluation using simulated and patient data. Quant Imaging Med Surg. 2020; 10(10):1917-1929. http://doi.org/l0.21037/qims-19-883
70. Wang S, Wang Q, Shao Y, et al. Iterative label denoising network: segmenting male pelvic organs in CT from 3D bounding box annotations. IEEE Trans Biomed Eng. 2020; 67(10):2710-2720. https://doi.org/10.1109/TBME.2020.2969608
71. Shi J, Zhang R, Guo L, Gao L, Ma H, Wang J. Discriminative feature network based on a hierarchical attention mechanism for semantic hippocampus segmentation. IEEE J Biomed Health Informatics. 2021; 25(2):504-513. https://doi.org/10.1109/JBHI.2020.2994114
72. Gao Y, Zhou M, Metaxas D N. UTNet: a hybrid transformer architecture for medical image segmentation. International Conference on Medical Image Computing and Computer-Assisted Intervention. Springer; 2021:61-71.
73. Isensee F, Jens P, Andre K, et al. nnu-net: self-adapting framework for u-net-based medical image segmentation. arXiv preprint. 2018. arXiv:1809.10486. https://doi.org/10.48550/arXiv.1809.10486
74. Wang Y, Zhao L, Song Z, Wang M. Organ at risk segmentation in head and neck CT images by using a two-stage segmentation framework based on 3D U-Net. IEEE Access. 2019; 7:144591-144602.
75. Men K, Geng H, Cheng C, et al. Technical Note: More accurate and efficient segmentation of organs-at-risk in radiotherapy with convolutional neural networks cascades. Med Phys. 2019; 46(1):286-292.
76. Tang M, Zhang Z, Cobzas D, Jagers and M, Jaremko J L. Segmentation-by-detection: a cascade network for volumetric medical image segmentation. IEEE 15th International Symposium on Biomedical Imaging. 2018:1356-1359. https://doi.org/10.1109/ISBI.2018.8363823
77. Guo D, Jin D, Zhu Z, et al. Organ at risk segmentation for head and neck cancer using stratified learning and neural architecture search. Proceedings of the IEEE/CVF Conference on Computer Vision and Pattern Recognition. 2020:4223-4232.
78. Gao Y, Huang R, Chen M, et al. FocusNet: imbalanced large and small organ segmentation with an end-to-end deep neural network for head and neck CT images. International Conference on Medical Image Computing and Computer-Assisted Intervention. 2019:829-838.
79. Liang S, Thung K H, Nie D, Zhang Y, Shen D. Multi-view spatial aggregation framework for joint localization and segmentation of organs at risk in head and neck CT images. IEEE Trans Med Imaging. 2020; 39(9):2794-2805.
80. Trullo R, Petitjean C, Nie D, Shen D, Ruan S. Joint segmentation of multiple thoracic organs in CT images with two collaborative deep architectures. In: Deep Learning in Medical Image Analysis and Multimodal Learning for Clinical Decision Support. 2017:21-29.
81. Kaldera H, Gunasekara S R, Dissanayake M B. Brain tumor classification and segmentation using faster R-CNN. Advances in Science and Engineering Technology International Conferences. 2019:1-6.
82. Kaldera H, Gunasekara S R, Dissanayake M B. MRI based glioma segmentation using deep learning algorithms. International Research Conference on Smart Computing and Systems Engineering. 2019:51-56.
83. Gunasekara S R, Kaldera HNTK, Dissanayake M B. A systematic approach for MRI brain tumor localization and segmentation using deep learning and active contouring. J Healthcare Eng. 2021; 2021:6695108.
84. Zhao Y, Li H, Wan S, et al. Knowledge-aided convolutional neural network for small organ segmentation. IEEE J Biomed Health Informatics. 2019; 23(4):1363-1373.
85. Lessmann N, Ginneken B V, Jong P D, et al. Iterative fully convolutional neural networks for automatic vertebra segmentation and identification. Med Image Anal. 2019; 53:142-155.
86. El Jurdi R, Petitjean C, Honeine P, Abdallah F. BB-UNet: u-Net with bounding box prior. IEEE J Select Top Signal Process. 2020; 14(6):1189-1198.
87. Bredell G, Christine T, Ender K. Iterative interaction training for segmentation editing networks. International Workshop on Machine Learning in Medical Imaging. 2018:363-370.
88. Wang G, Li ZuluagaMA, Pratt R, et al. Interactive medical image segmentation using deep learning with image-specific fine tuning. IEEE Trans Med Imaging. 2018; 37(7):1562-1573.
89. Wang G, Li W, Zuluaga A, et al. DeepIGeoS: a deep interactive geodesic framework for medical image segmentation. IEEE Trans Pattern Anal Mach Intell. 2019; 41(7):1559-1572.
90. Liao X, Li W, Xu Q, et al. Iteratively-refined interactive 3D medical image segmentation with multi-agent reinforcement learning. Proceedings of the Conference on Computer Vision and Pattern Recognition. 2020:9394-9402.
91. Agrawal V, Udupa J K, Tong Y, Torigian D A. BRR-Net: a CNN-RNN tandem architecture for automatic body region localization in CT images. Med Phys. 2020; 47(10):5020-5031. https://doi.org/10.1002/MP.14439
92. Liu T, Pan J, Torigian D A, et al. ABCNet: a new efficient 3D dense-structure network for segmentation and analysis of body tissue composition on body-torso-wide CT images. Med Phys. 2020; 47(7):2986-2999.
93. Liu S, Qi L, Qin H, Shi J, Jia J. Path aggregation network for instance segmentation. Proceedings of the IEEE Conference on Computer Vision and Pattern Recognition. 2018:8759-8768.
94. Fu J, Liu J, Tian H, et al. Dual attention network for scene segmentation. Proceedings of the IEEE Conference on Computer Vision and Pattern Recognition. 2019:3146-3154.
95. Wang Y, Deng Z, Hu X, et al. Deep attentional features for prostate segmentation in ultrasound. International Conference on Medical Image Computing and Computer-Assisted Intervention. 2018:523-530.
96. Zhao H, Jia J, Koltun V. Exploring self-attention for image recognition. Proceedings of the IEEE/CVF Conference on Computer Vision and Pattern Recognition. 2020:10076-10085.
97. Lin T Y, Goyal P, Girshick R, He K, Dollir P. Focal loss for dense object detection. IEEE Trans Pattern Anal Mach Intell. 2020; 42(2):318-327. https://doi.org/10.1109/TPAMI.2018.2858826
98. Li J, Udupa J K, Tong Y, Wang L, Torigian D A. LinSEM: linearizing segmentation evaluation metrics for medical images. Med Image Anal. 2020; 60:101601. https://doi.org/10.1016/j.media.2019.101601
99. Raudaschl P F, Zaffino P, Sharp G C, et al. Evaluation of segmentation methods on head and neck CT: auto-segmentation challenge 2015. Med Phys. 2017; 44(5):2020-2036.
100. Lambert Z, Petitjean C, Dubray B, Ruan S. SegTHOR: segmentation of thoracic organs at risk in CT images. 2020 Tenth International Conference on Image Processing Theory, Tools and Applications. 2020:1-6.
101. Brouwer C L, Steenbakkers R J, Bourhis J, et al. CT-based delineation of organs at risk in the head and neck region: dAHANCA, EORTC, GORTEC, HKNPCSG, NCIC CTG, NCRI, NRG oncology and TROG consensus guidelines. Radiother Oncol. 2015; 117:83-90.
102. Brouwer C L, Steenbakkers R J, Bourhis J, et al. CT-based delineation of organs at risk in the head and neck region: dAHANCA, EORTC, GORTEC, HKNPCSG, NCIC CTG, NCRI, NRG oncology and TROG consensus guidelines. Radiother Oncol. 2015; 117:83-90. Supplement Material.
103. He K, Zhang X, Ren S, Sun J. Deep residual learning for image recognition. Proceedings of the IEEE Conference on Computer Vision and Pattern Recognition. 2016:770-778.
104. Fu J, Liu J, Tian H, et al. Dual attention network for scene segmentation. Proceedings of the IEEE Conference on Computer Vision and Pattern Recognition. 2019:3146-3154.
105. Udupa, J. K., Odhner, D., Torigian, D. A., Tong, Y.: Applications of automatic anatomy recognition in medical tomographic imagery based on fuzzy anatomy models, patent application submitted in 2014, Penn Docket: 14-7011.
106. Udupa J K, Agrawal V, Tong Y, Torigian D A. BRR-Net: A highly accurate deep learning network for automatic body region recognition in torso-wide CT Images, IP disclosure, submitted to PCI on 6-12-2019, Penn Docket: 19-9062.
107. YOLOv4: Optimal Speed and Accuracy of Object Detection.

### Description of Computer Features for Implementing the Disclosure

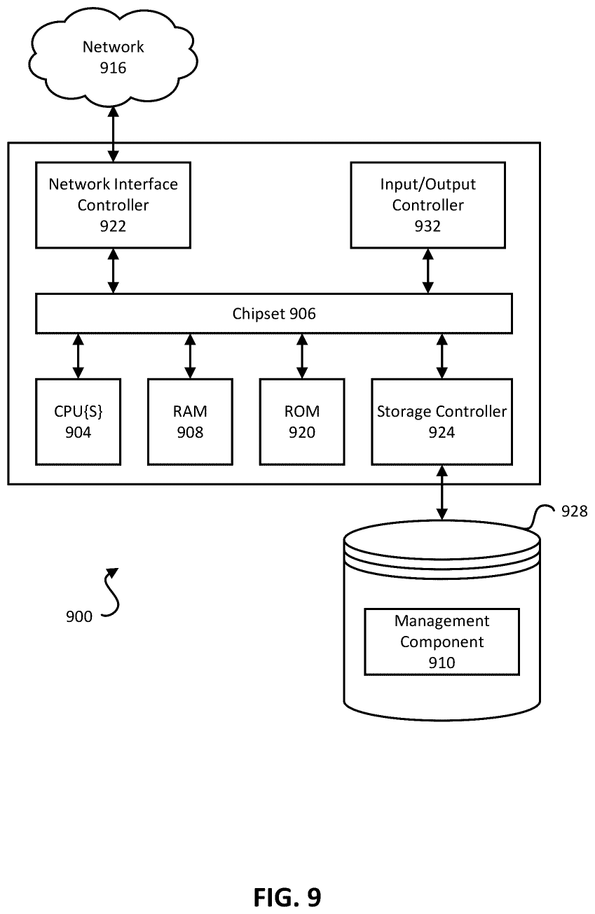

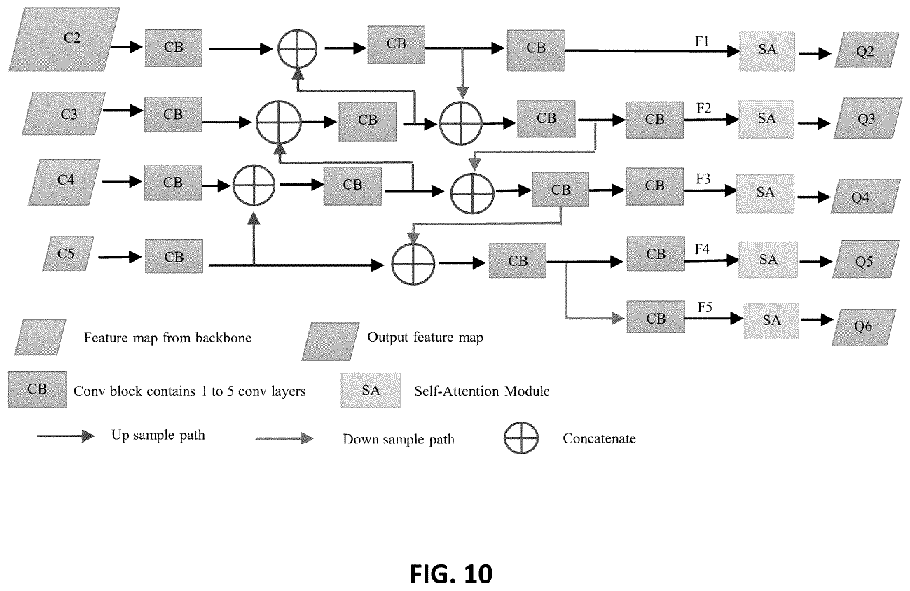

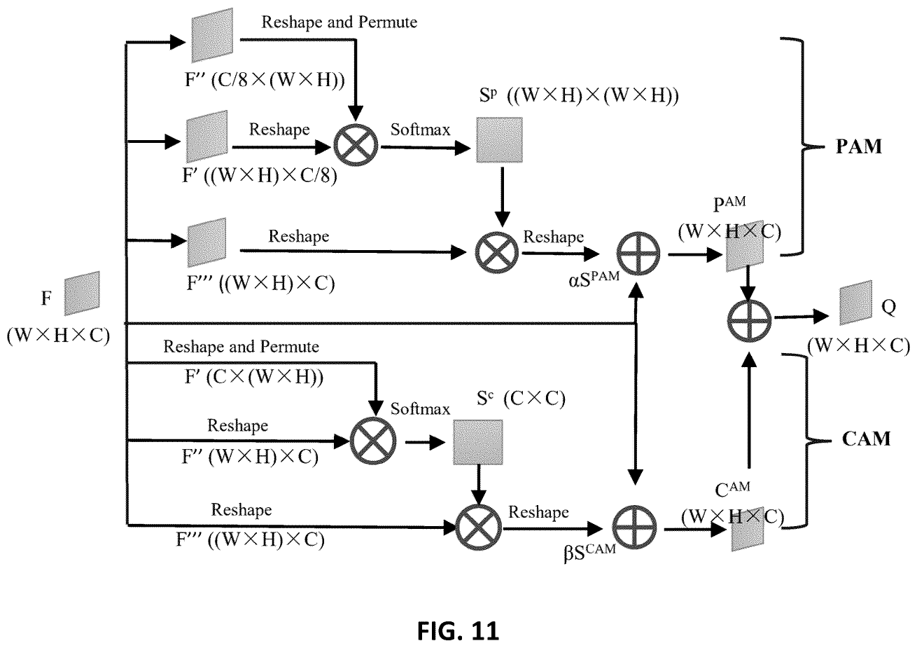

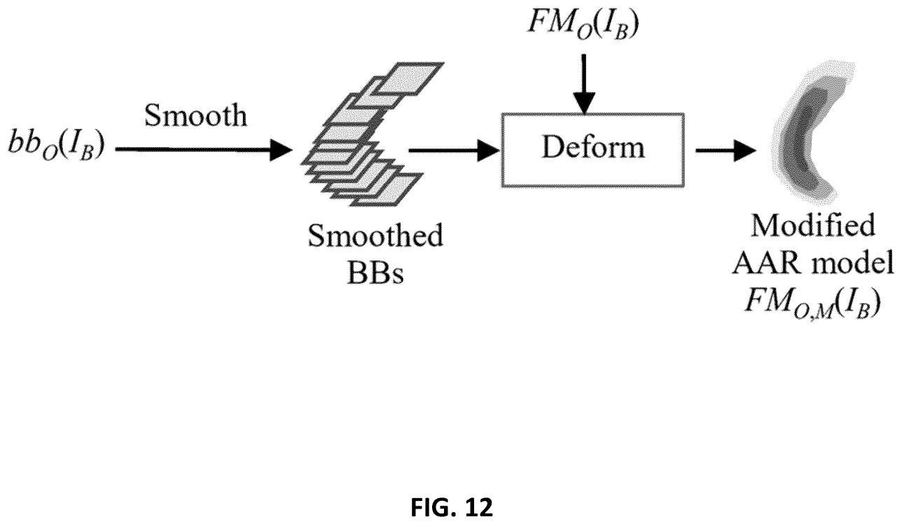

FIG. 9 depicts a computing device that may be used in various aspects, such as the servers, modules, and/or devices depicted in FIG. 1, FIG. 2, FIG. 10, FIG. 11, and FIG. 12. The computer architecture shown in FIG. 9 shows a conventional server computer, workstation, desktop computer, laptop, tablet, network appliance, PDA, e-reader, digital cellular phone, or other computing node, and may be utilized to execute any aspects of the computers described herein, such as to implement the methods described in relation to FIG. 1, FIG. 2, FIG. 10, FIG. 11, and FIG. 12.

The computing device 900 may include a baseboard, or “motherboard,” which is a printed circuit board to which a multitude of components or devices may be connected by way of a system bus or other electrical communication paths. One or more central processing units (CPUs) 904 may operate in conjunction with a chipset 906. The CPU(s) 904 may be standard programmable processors that perform arithmetic and logical operations necessary for the operation of the computing device 900.

The CPU(s) 904 may perform the necessary operations by transitioning from one discrete physical state to the next through the manipulation of switching elements that differentiate between and change these states. Switching elements may generally include electronic circuits that maintain one of two binary states, such as flip-flops, and electronic circuits that provide an output state based on the logical combination of the states of one or more other switching elements, such as logic gates. These basic switching elements may be combined to create more complex logic circuits including registers, adders-subtractors, arithmetic logic units, floating-point units, and the like.

The CPU(s) 904 may be augmented with or replaced by other processing units, such as GPU(s) 905. The GPU(s) 905 may comprise processing units specialized for but not necessarily limited to highly parallel computations, such as graphics and other visualization-related processing.

A chipset 906 may provide an interface between the CPU(s) 904 and the remainder of the components and devices on the baseboard. The chipset 906 may provide an interface to a random access memory (RAM) 908 used as the main memory in the computing device 900. The chipset 906 may further provide an interface to a computer-readable storage medium, such as a read-only memory (ROM) 920 or non-volatile RAM (NVRAM) (not shown), for storing basic routines that may help to start up the computing device 900 and to transfer information between the various components and devices. ROM 920 or NVRAM may also store other software components necessary for the operation of the computing device 900 in accordance with the aspects described herein.

The computing device 900 may operate in a networked environment using logical connections to remote computing nodes and computer systems through local area network (LAN) 916. The chipset 906 may include functionality for providing network connectivity through a network interface controller (NIC) 922, such as a gigabit Ethernet adapter. A NIC 922 may be capable of connecting the computing device 900 to other computing nodes over a network 916. It should be appreciated that multiple NICs 922 may be present in the computing device 900, connecting the computing device to other types of networks and remote computer systems.

The computing device 900 may be connected to a mass storage device 928 that provides non-volatile storage for the computer. The mass storage device 928 may store system programs, application programs, other program modules, and data, which have been described in greater detail herein. The mass storage device 928 may be connected to the computing device 900 through a storage controller 924 connected to the chipset 906. The mass storage device 928 may consist of one or more physical storage units. A storage controller 924 may interface with the physical storage units through a serial attached SCSI (SAS) interface, a serial advanced technology attachment (SATA) interface, a fiber channel (FC) interface, or other type of interface for physically connecting and transferring data between computers and physical storage units.

The computing device 900 may store data on a mass storage device 928 by transforming the physical state of the physical storage units to reflect the information being stored. The specific transformation of a physical state may depend on various factors and on different implementations of this description. Examples of such factors may include, but are not limited to, the technology used to implement the physical storage units and whether the mass storage device 928 is characterized as primary or secondary storage and the like.

For example, the computing device 900 may store information to the mass storage device 928 by issuing instructions through a storage controller 924 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computing device 900 may further read information from the mass storage device 928 by detecting the physical states or characteristics of one or more particular locations within the physical storage units.

In addition to the mass storage device 928 described above, the computing device 900 may have access to other computer-readable storage media to store and retrieve information, such as program modules, data structures, or other data. It should be appreciated by those skilled in the art that computer-readable storage media may be any available media that provides for the storage of non-transitory data and that may be accessed by the computing device 900.

By way of example and not limitation, computer-readable storage media may include volatile and non-volatile, transitory computer-readable storage media and non-transitory computer-readable storage media, and removable and non-removable media implemented in any method or technology. Computer-readable storage media includes, but is not limited to, RAM, ROM, erasable programmable ROM (“EPROM”), electrically erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage, other magnetic storage devices, or any other medium that may be used to store the desired information in a non-transitory fashion.

A mass storage device, such as the mass storage device 928 depicted in FIG. 9, may store an operating system utilized to control the operation of the computing device 900. The operating system may comprise a version of the LINUX operating system. The operating system may comprise a version of the WINDOWS SERVER operating system from the MICROSOFT Corporation. According to further aspects, the operating system may comprise a version of the UNIX operating system. Various mobile phone operating systems, such as IOS and ANDROID, may also be utilized. It should be appreciated that other operating systems may also be utilized. The mass storage device 928 may store other system or application programs and data utilized by the computing device 900.

The mass storage device 928 or other computer-readable storage media may also be encoded with computer-executable instructions, which, when loaded into the computing device 900, transforms the computing device from a general-purpose computing system into a special-purpose computer capable of implementing the aspects described herein. These computer-executable instructions transform the computing device 900 by specifying how the CPU(s) 904 transition between states, as described above. The computing device 900 may have access to computer-readable storage media storing computer-executable instructions, which, when executed by the computing device 900, may perform the methods described in relation to FIG. 1, FIG. 2, FIG. 10, FIG. 11, and FIG. 12.

A computing device, such as the computing device 900 depicted in FIG. 9, may also include an input/output controller 932 for receiving and processing input from a number of input devices, such as a keyboard, a mouse, a touchpad, a touch screen, an electronic stylus, or other type of input device. Similarly, an input/output controller 932 may provide output to a display, such as a computer monitor, a flat-panel display, a digital projector, a printer, a plotter, or other type of output device. It will be appreciated that the computing device 900 may not include all of the components shown in FIG. 9, may include other components that are not explicitly shown in FIG. 9, or may utilize an architecture completely different than that shown in FIG. 9.

As described herein, a computing device may be a physical computing device, such as the computing device 900 of FIG. 9. A computing node may also include a virtual machine host process and one or more virtual machine instances. Computer-executable instructions may be executed by the physical hardware of a computing device indirectly through interpretation and/or execution of instructions stored and executed in the context of a virtual machine.

### Additional Examples—Section A

### 10|Standardized Definition of Objects

Standard widely accepted definitions are not available in medical literature, and arriving at a proper definition of objects is not a straightforward process. From the perspective of AI, the definition should be (i) anatomically relevant for the application and valid in all (or most) patient cases; (ii) feasible to implement; and (iii) able to achieve high recognition accuracy. For illustration, consider the process of defining the neck (Nk) body region. See Table 15, which includes definition of the thorax body region as well. In the previous sense of qualitative understanding, in medical practice, body regions are generally identified in the cranio-caudal direction partitioned by axial (transverse) planes,⁴ and the neck region is understood to be roughly in the region inferior to the skull base and superior to the level of the clavicles. To arrive at a computationally feasible definition satisfying the previous conditions, we define the superior boundary as the axial level that is most superior among the superior-most aspects of the left and right mandibular condyles and coronoid processes. The inferior boundary is defined as the axial level at which the superior vena cava branches into left and right brachiocephalic veins.

This definition is anatomically relevant for the RT application as the defined body region fully covers all objects commonly required to be segmented for this application. It is valid as the body region is well defined without ambiguities. Note that if we used just the superior-most aspect of the mandibular condyles for defining the superior boundary, the definition may be invalid or ambiguous, as depending on the neck tilt (bent forward, straight, or bent backward), either the mandibular coronoid processes or the mandibular condyles on either side may become the superior-most aspect of the mandible, such that part of the mandibular coronoid processes may fall outside of the body region. This definition is feasible to implement manually and automatically. As the superior boundary is well defined and easily recognized in the image, the variability in its manual recognition can be in the order of one slice. The inferior boundary on the other hand may be a bit more ambiguous due to the digital ambiguity of when to call the superior vena cava as really branching. This ambiguity can be minimized by defining the branch point as the level at which the superior vena cava changes from a roughly circular cross section to an oval or lemniscate shape. Given the digital ambiguity, manual recognition variability for inferior boundary may be in the order of two slices depending on slice spacing. For auto-recognition as well, the two boundaries are feasible computationally, the inferior boundary being a bit more challenging than the superior as for manual recognition.⁹¹

Similar to body regions, we define precisely every object included in each body region in the same spirit. This definition should also satisfy the previous conditions specified for body regions. In addition, the regions o be included and excluded within the 3D region of the defined object are to be clearly specified in the definition. As an example, Table 14 illustrates the definition of supraglottic/glottic larynx in the neck body region. When available, we employ existing guidelines for developing the precise object definitions. For example, for our application of auto-contouring for RT planning, we adopted the contouring guidelines provided in Refs. [101, 102].

TABLE 13
Body region definitions for neck and thorax and object definition
example for supraglottic/glottic larynx

| | | |
|---|---|---|
| Neck | Definition | Region of the body within the cervical skin outer |
| | Superior boundary NkS(I) | boundary between the superior and inferior axial |
| | Inferior boundary NkI(I) | boundary levels defined next |
| | Image display | Superior-most aspect of the entire mandible, including |
| | | the left and right mandibular condyles and |
| | | mandibular coronoid processes |
| | | The superior aspect of the superior vena cava where it |
| | | branches into left and right brachiocephalic veins |
| | | Use bone window for defining the superior boundary |
| | | and soft-tissue window for identifying the inferior |
| | | boundary |
| Thorax | Definition | Region of the body within the thoracic skin outer |
| | | boundary between the superior and inferior axial |
| | Superior boundary ThS(I) | levels defined next |
| | Inferior boundary ThI(I) | Axial slice located 15-mm superior to the lung apices |
| | Image display | Axial slice located 5-mm inferior to the lung bases |
| | | Use lung window for defining both boundaries |
| Supraglottic/ | Definition | The larynx is composed of supraglottic, glottic, and |
| glottic larynx | | subglottic components. Here, we consider only the |
| | | supraglottic and glottic components, excluding the |
| | Superior boundary | thyroid, arytenoid, and cricoid cartilages, but |
| | Inferior boundary | including the gas-filled lumen |
| | | The superior aspect of the epiglottis |
| | Anterior boundary | |
| | Posterior boundary | The inferior edge of the anterior aspect of the thyroid |
| | | cartilage (fully including the true vocal cords) |
| | Image display | The posterior border of hyoid bone, strap muscles, and |
| | | posterior border of the thyroid cartilage |
| Neck | Definition | Region of the body within the cervical skin outer |
| | Superior boundary NkS(I) | boundary between the superior and inferior axial |
| | Inferior boundary NkI(I) | boundary levels defined next |
| | Image display | Superior-most aspect of the entire mandible, including |
| | | the left and right mandibular condyles and |
| | | mandibular coronoid processes |
| | | The superior aspect of the superior vena cava where it |
| | | branches into left and right brachiocephalic veins |
| | | Use bone window for defining the superior boundary |
| | | and soft-tissue window for identifying the inferior |
| | | boundary |
| | | The posterior borders of the epiglottis and aryepiglottic |
| | | folds, and the anterior borders of |
| | | orohypopharyngeal constrictor muscle, arytenoid |
| | | cartilages, and posterior cricoid cartilage |
| | | Use soft-tissue window for tracing the ground-truth |
| | | boundaries |

In our implementation of the HI system, we first develop the definitions, then check the feasibility of their implementation, and go back and modify the definitions if needed. Once finalized, a document is prepared for each body region that delineates all definitions with image examples, prescribes display window setting for each object (which influences the ground-truth [GT] contours traced), and tools to use for actual GT tracing. Precise and consistent tracing of GT boundaries is extremely crucial for the effectiveness of the HI system. This is as important for encoding NI as for precisely evaluating the segmentation accuracy⁵ of the HI system. In this work, GT contouring and recognition of region boundary slices were performed by mostly individuals without formal medical training. However, they all went through rigorous training conducted by experts.6 After completing an initial set of a few cases, the results were examined by the experts and corrective changes were indicated. The GT operators kept detailed notes on any unusual anatomic situation/image characteristics/ambiguities encountered, which were clarified by experts subsequently. Two among the trained and most experienced operators then examined all GT results as an overall check to ensure high GT quality.

### A.2 Object Recognition Refinement Via Deep Learning (DL-R)

### A.2.1 Backbone Network

The input image is processed through the backbone network to output preliminary feature maps. To make full use of pretrained model weights, one of the popular networks ResNet-101¹⁰³ is adopted in this work. Table 16 shows the architecture of the backbone network, where the major deviation from ResNet-101 is C6, which is extracted from C5 with stride convolution operations, which contains information at a more abstract level. C2-C5 denote the conv2, conv3, conv4, and conv5 output feature maps from the last layer of each backbone block, respectively. Finally, all the previous layers along with the extra layer C6 are input into the neck network. It is worth noting that as the previous AAR-R module already provides a rough position for each object, the high-resolution information from C1 is unnecessary for the subsequent neck and head networks.

TABLE 14
Architecture of the Backbone network

| Layer | Down-sampling size | Convolutional operations |
|---|---|---|
| C1 | 2 × 2 | 7, 64, stride 2 |
| C2 | 4 × 4 | 3 × 3 max pool, stride 2 |
| | | $[ \begin{matrix}11 , & 64 \\ 33 , & 64 \\ 11 , & 256\end{matrix} ] \times 3$ |
| C3 | 8 × 8 | 3 × 3 max pool, stride 2 |
| | | $[ \begin{matrix}1 \times 1 , & 128 \\ 3 \times 3 , & 128 \\ 1 \times 1 , & 512\end{matrix} ] \times 4$ |
| C4 | 16 × 16 | 3 × 3 max pool, stride 2 |
| | | $[ \begin{matrix}1 \times 1 , & 256 \\ 3 \times 3 , & 256 \\ 1 \times 1 , & 1024\end{matrix} ] \times 23$ |
| C5 | 32 × 32 | 3 × 3 max pool, stride 2 |
| | | $[ \begin{matrix}1 \times 1 , & 512 \\ 3 \times 3 , & 512 \\ 1 \times 1 , & 2048\end{matrix} ] \times 3$ |
| C6 | 64 × 64 | 3 × 3 1024, stride 2 |

Note:
Building blocks are shown in brackets, with the numbers of blocks stacked.

### A.2.2 Neck Network

FIG. 10 shows the complete structure of our neck network. This design is based on our desire that accurate recognition of non-sparse objects also requires detailed local information, especially at the object edges and corners. As shown in FIG. 10, each building block takes a higher resolution feature map and a coarser map through lateral connections and generates the new feature map Fi.

FIG. 10 shows a complete structure of the neck network.

FIG. 11 shows the structure of the SA module.

We apply Dual Attention Network¹⁰⁴ to adaptively integrate local features with their global dependencies further for each Fi. We call this attention network Self Attention (SA) module given that it involves attention inside each feature map Fi. The SA module consists of a Position Attention Module (PAM) and a Channel Attention Module (CAM) to fully process the feature maps, as depicted in FIG. 11.

For PAM, F is an input feature map of size (W, H, C) from the previous step, denoting the width, height, and number of channels, respectively. To make FIG. 11 clearer, we replace Fi by F. F is passed through a convolution layer to generate two new feature maps F′ and F″ of size (W, H, C/8). They are reshaped to size ((W×H), C/8) and (C/8, (W×H)). Then a matrix multiplication is performed between the two feature maps and a softmax layer is attached to generate the spatial attention map Sp with size ((W×H), (W×H)). Meanwhile, F is passed through another convolution layer and reshaped into F′″ of size ((W×H), C). Then a matrix multiplication is performed between F″ and Sp to obtain a new ((W×H), C) feature map. Subsequently, a map S^{pam} is generated with the shape as the input feature map F by a reshape operation. Finally, a trainable parameter a is attached to Spam and an element-wise sum operation with feature map F is performed. The attention feature map P^{AM} of PAM is generated as P^{AM}=αS^{pam}+F, +where α is initialized to 0.

For CAM, the networλ is similar to PAM but more simplified. Different from PAM, without the convolution operation, input feature map F is reshaped into F′ and F″ of size (C, (W×H)) and ((W×H), C), respectively. Then a matrix multiplication is performed between them and a softmax operation is followed to generate SC which has size (C, C). Meanwhile, F is passed through another convolution layer and reshaped into F″ of size ((W×H), C). Then a matrix multiplication is performed between S^{C} and F′″ to obtain a new feature map of size ((W×H), C). Subsequently, a map S^{CAM} is generated with the shape as the input feature map F by a reshape operation. Finally, the attention feature map C^{AM} of CAM is generated in the same way as PAM: C^{AM}=βS^{CAM}+F, where β is a trainable parameter and initialized to 0. Finally, the output of the SA module is the sum of the two attention feature maps: Q=P^{AM}+C^{AM}.

### A.2.3 Head Network

In medical images, target organ characteristics such as position, size, shape, and sparsity are relatively fixed and known a priori. The DL-R model recognizes multiple objects simultaneously by dividing target objects into two groups according to their sparsity. By observing the structure of the DL-R network shown in FIG. 2 of the manuscript, prediction maps denoted by Q2-Q6 are generated by the neck network, which have different resolutions and are sensitive to organs of different sizes.

In order to improve the accuracy and efficiency of DL-R, organs with different sparsities take different prediction maps to perform classification. On the one hand, non-sparse organs are recognized using maps Q4, Q5, and Q6 associated with anchors with base sizes 32×32, 64×64, and 128×128, respectively. Larger receptive fields and semantically stronger information from higher level layers are crucial for recognizing non-sparse organs. On the other hand, sparse organs are recognized using maps Q2, Q3, and Q4 associated with anchors with base sizes 8×8, 16×16, and 32×32, respectively. More detailed structural and spatial characteristics are important to recognize sparse organs. In order to get a denser prediction, recognition candidate cells are then augmented with anchors with different aspect ratios and scale based on anchor base sizes. Lastly, the head network that contains only convolution layers predicts the category and location of target organs based on corresponding prediction maps and anchors.

TABLE 15
Key details pertaining to the near-normal data sets used in this
study for automatic anatomy recognition (AAR) model building

| Parameters | Neck | Thorax |
|---|---|---|
| Number | From PET/CT: 18 | From PET/CT: 39 |
| of cases | Diagnostic CT: 21 | Diagnostic CT: 19 |
| | Total: 39 | Total: 58 |
| Patient | Gender: male-34; female-5 | Gender: male-28; |
| demographics | Age: 33-76; mean-58.26; | female-30 |
| | SD-9.72 | Age: 30-85; mean-59.65; SD-14.74 |
| | Race: unknown-39 | Race: White-11; African-3; Asian-1; |
| | Ethnicity: unknown-39 | other-2; unknown-41 |
| | | Ethnicity: unknown-58 |
| Scanner | Philips: 38 | Philips: 49 |
| types | Siemens: 1 | Siemens: 9 |
| Image | Contrast-enhanced: yes-21; | Contrast-enhanced: yes-2; no-56 |
| parameter | no-18 | Image size: 512 × 512 × (44-96) |
| | Image size: 512 × 512 × [91-148] | Pixel size: 0.98-1.52 mm |
| | Pixel size: 0.98-1.17 mm | Slice spacing: 3-5 mm |
| | Slice spacing: 1.5-3 mm | |

Abbreviation:
CT, computed tomography;
PET, positron emission tomography.

FIG. 12 is an illustration of the morphing process

### A.3 Object Model Morphing Guided by Bounding Boxes (MM)

As shown in FIG. 12, the MM module smooths bb_{O}(I_{B}) to create smoothed BBs and then performs deformation on FM_{O}(I_{B}) based on the smoothed BBs to achieve the final modified FM_{O,M}(I_{B}). The process runs as follows.

We first compute the center (x′ᵢ, y′ᵢ) of the 2D BBs in bb_{O}(I_{B}) in all n slices of object O. We expect the corresponding “real” centers (xᵢ, yᵢ) for O to form a smooth line along the third dimension due to the fact that object shapes change smoothly from slice-to-slice. Expressing the deviation of (x′ᵢ, y′ᵢ) from(xᵢ, yᵢ) within the slice plane to be (wᵢ, vᵢ),we have

$${x}_{i}^{′} = {x}_{i} + {w}_{i} \\ {y}_{i}^{′} = {y}_{i} + {v}_{i}$$

We fit a smooth curve to the computed centers by using a quadratic approximation:

$${x}_{i} = {a}_{1} {i}^{2} + {b}_{1} i + {c}_{1} \\ {y}_{i} = {a}_{2} {i}^{2} + {b}_{2} i + {c}_{2}$$

where i=1, . . . , n is the slice number, and a1, b1, c1, a2, b2, and c2 are unknown parameters. We estimate them by minimizing the mean squared error as

$${\hat{a}}_{1} , {}^{\hat{}} {b}_{1} , {}^{\hat{}} {c}_{1} = arg {min}_{{a}_{1} , {b}_{1} , {c}_{1}} {\sum }_{i = 1}^{n} {( {x}_{i} - {a}_{1} {i}^{2} - {b}_{1} i - {c}_{1} )}^{1} \\ {\hat{a}}_{2} , {}^{\hat{}} {b}_{2} , {}^{\hat{}} {c}_{2} = arg {min}_{{a}_{2} , {b}_{2} , {c}_{2}} {\sum }_{i = 1}^{n} {( {y}_{i} - {a}_{2} {i}^{2} - {b}_{2} i - {c}_{2} )}^{2}$$

After estimating the unknown parameters, the center coordinates on the smooth line in each slice are estimated from the previous equations.

### A.4 Acquiring Near-Normal Data Sets for Model Building

The selection criterion for near-normal data sets was that for the body region considered the patient images appeared radiologically normal with exception of minimal incidental focal abnormalities such as cysts and small pulmonary nodules. Images with severe motion/streak artifacts or other limitations were excluded from consideration. Patient cases were selected from two sources: whole-body or neck plus body-torso PET/CT scans as well as diagnostic CT scans acquired with or without intravenous contrast material. From PET/CT scans, only CT data sets were used when near-normal. Table 17 summarizes key parameters related to these scans for the two body regions. AAR models FAM(B) were built separately for each body region from these data sets.

### Additional Examples Section B—Additional Methods

### Additional Applications and Illustrations for the Hybrid Intelligence Patent Application

The HI system related publications demonstrated the idea using planning or simulation computed tomography (CT) data sets for the application of radiation therapy (RT) planning of the cancers involving the neck and thorax body regions. In our new work not contained in those publications, we have extended the HI system to other body regions, image modalities, and applications, as summarized below.

### 1. Magnetic Resonance Imaging (MRI)

The HI approach can be used by pediatric specialists in disease diagnosis, treatment planning, treatment response assessment, and surveillance assessment of children suffering from various forms of respiratory ailments. For these applications, we acquire dynamic MRI (dMRI) while the patient is lying in the scanner breathing normally. The dMRI technique does not require holding breath or any other maneuvers such as taking a deep breath or forced exhalation. A slice image is acquired at each sagittal location across the entire chest while the child is breathing freely and naturally. Below, we demonstrate two applications of the HI method utilizing dMRI in the pediatric population.

a. Segmentation of Thoraco-Abdominal Organs to Assess Patients with Thoracic Insufficiency Syndrome (TIS).

The HI approach can be used to first segment automatically thoraco-abdominal organs such as the left and right lungs individually, left and right kidneys individually, and spleen via dMRI and then to analyze their volumes, motion, and architecture to study the impact of respiratory restrictions on their form and function. By deriving such quantitative measures from pre-treatment and post-treatment dMRI, treatment can be planned effectively, and the effect of treatment can be ascertained [1-3]. These earlier demonstrations used manual segmentations of the dMRI.

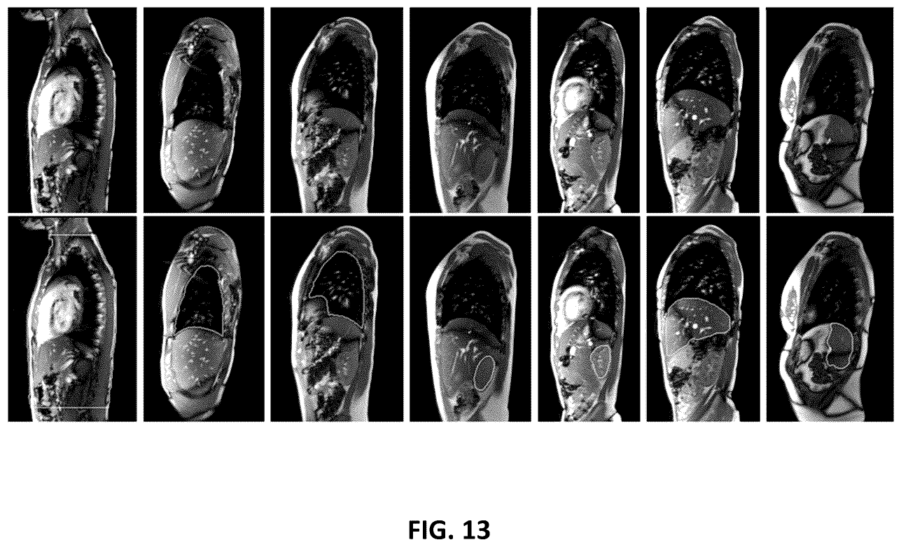

The adaptation of the HI system from the original planning CT studies to dMRI involved the following additional developments. (i) Since the appearance of dMRI is very different from CT, all hyperparameters of the different steps—AAR-R, DL-R, and DL-D—had to be re-optimized for best performance. (ii) The dMRI acquisitions are in sagittal slices while CT acquisitions are in axial slices. This meant that the BRR module for recognizing the thoracic body region via axial slices had to be replaced. We created a new module in place of BRR to automatically identify a horizontal line superiorly in the sagittal slice that would correspond to the apex of the lungs and another horizontal line inferiorly that would correspond to the inferior aspect of the kidneys. From the superior most of all these lines in the dMRI slices, a superior axial slice defining the superior axial boundary of the thorax was determined, and from the inferior most of the lines the inferior axial slice defining the inferior boundary of the thoraco-abdominal region was determined. (iii) Since the MRI images are dynamic, representing the thoraco-abdominal region over one respiratory cycle, the HI system was modified in several ways to handle this 4D image compared to the static 3D CT image. All modules were trained on the 3D images representing the end expiration (EE) phase. For DL-R and DL-D, further changes included using the region defined by the segmentation at time point (phase) t as the recognition result for the 3D image at time point t+1, etc. to determine the best strategy for segmenting the entire 4D image. Some representative results are shown in FIG. 13 illustrating auto-segmentation of skin, right lung, left lung, right kidney, left kidney, liver, and spleen. The Dice coefficient achieved for the different objects ranged from 0.82 to 0.96. No methods currently exist for the 3D and 4D segmentation of these organs in dMRI.

A very preliminary conference paper appeared on this extension of the HI system for this application on Apr. 3, 2023 [4] which showed results only for the two lungs.

b. Segmentation and Analysis of the Motion and Shape of the Diaphragm to Assess Patients with TIS.

FIG. 13 shows examples of thoraco-abdominal organ segmentation in dynamic MRI using the HI approach. A slice from a dMRI study (top row) and the segmented organ overlaid on the slice (bottom row). From left to right: Skin, Right Lung, Left Lung, Right Kidney, Left Kidney, Liver, and Spleen.

The diaphragm is a vital structure in the respiratory mechanics of the chest. The HI approach can be used to automatically segment each hemi-diaphragm separately via dMRI acquisitions. Once the segmentation is given, the 3D motion and shape of the hemi-diaphragms during the respiratory cycle can be quantified, as illustrated in [5] by using manually segmented hemi-diaphragms. By deriving such quantitative measures from pre-treatment and post-treatment dMRI, treatment can be planned effectively, and the effect of treatment can be ascertained.

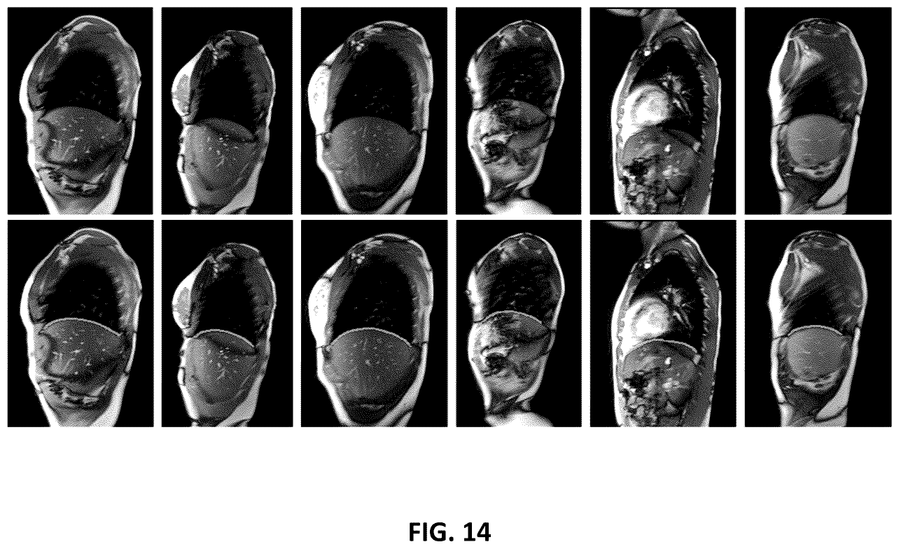

The adaptation of the HI system from the original planning CT studies to dMRI for the auto-segmentation of the diaphragm involved the following additional developments. (i) Same as 1a(ii) above. (ii) The DL-R module was replaced by a simple strategy as follows. Since the lungs have already been segmented (following the method in Ta), we used their segmentations to automatically determine a rectangular bounding box region of interest (ROI) for the entire diaphragm. This is facilitated by the fact that the diaphragm appears below the inferior boundary of the lungs in the slices. This ROI was designed to be a bit larger than the tight-fitting ROI since the DL module requires some space around the diaphragm boundary to collect the needed background information to segment the diaphragm accurately. (iii) The hyperparameters of the DL module were optimally set to tailor its performance to segment the hemi-diaphragms which are very thin and slender compared to the other organs. The DL module first segments the whole diaphragm. An additional modification (sub-module) is incorporated into DL to separate the right and the left hemi-diaphragms automatically as follows. We designed and trained a Recurring Neural Network (RNN) to detect the mid-sagittal slice from the sequence of sagittal slices beginning from the right lateral edge of the chest and going toward the left lateral edge. The RNN learned the pattern of changes in the dMRI image in this sequence to accurately detect the mid-sagittal slice. In our test, the accuracy of this detection was ˜1 slice, which is similar to the variation that would be found when experts localize the mid-sagittal slice. The error in auto-delineation of the hemi-diaphragms in terms of distance from reference true boundary has been found to be ˜1.6 mm and 2.1 mm for the right and left hemi-diaphragms, respectively. FIG. 14 shows some examples of auto-segmentations of the hemi-diaphragms in dMRI in 3 different studies.

FIG. 14 shows examples of hemi-diaphragm segmentation in dynamic MRI using the HI approach. A slice from three dMRI studies (top row) and the segmented hemi-diaphragms overlaid on the slice (bottom row). The first three examples are for Right Hemi-Diaphragm followed by the next three examples for Left Hemi-Diaphragm.

### 2. PET/CT Imaging

a. Neck and Thorax Organ Segmentation for Disease Quantification.

The HI system can be utilized to segment various organs of the neck and thorax in low-dose CT images of positron emission tomography/computed tomography (PET/CT) acquisitions obtained after administration of a radiotracer of interest (e.g., ¹⁸F-fluorodeoxyglucose (FDG)). The segmentation information can then be transferred to the PET images, and subsequently the burden of metastatic cancer in the whole body region and individual organs can be quantified. The quantification method is described in [6] which was based on manual segmentation of the organs. Based on prior knowledge of the normal distribution of PET radiotracer activity within that organ, this method of quantification first estimates what the total normal metabolic activity would be within the organ if the organ were to be normal, and then subtracts this amount from the actual total metabolic activity observed in the organ in the PET image.

We adapted the HI system from the original planning CT studies to the low-dose CT of PET/CT acquisitions for disease quantification. Our adaptation of the HI system included the following modifications. (i) Since the appearance and image characteristics of low-dose CT are somewhat different from the planning CT used in the original HI system, all hyperparameters of the different steps—AAR-R, DL-R, and DL-D—were re-optimized for best performance. (ii) We re-trained AAR-R, DL-R, and DL-D modules on the low-dose CT images. Using data sets from 34 normal subjects and 58 patients with different types of cancer, we demonstrated that total lesion burden (TLB) can be quantified automatically using 9 objects as examples on FDG-PET/CT images. The objects considered were: Left Lung, Right Lung, Thoracic Esophagus, Thoracic skeleton, Right Hilar Lymph Node Zone, Left Hilar Lymph Node Zone, Right Axillary Lymph Node Zone, Left Axillary Lymph Node Zone, and Mediastinal Lymph Node Zone. Establishing ground truth TLB is impossible in patient cases since manual outlining of lesions is highly unreliable due to the fuzzy boundaries of the lesions and the hazy nature of many lesions which manifest themselves as a haze or cloud of abnormal signal without perceivable boundaries. Therefore, we evaluated the method on normal subjects for human testing, where we expect TLB to be ˜0 and there is no need for human outlining of lesions, and also on phantoms containing artificially created “lesions” of differing but known size and of differing but known radiotracer activity [7]. Our evaluation showed that in phantoms, the error of our system in TLB varied from 0.8% to 5.4% for lesions in phantoms and from 1.7% to 5.2% in normal subjects.

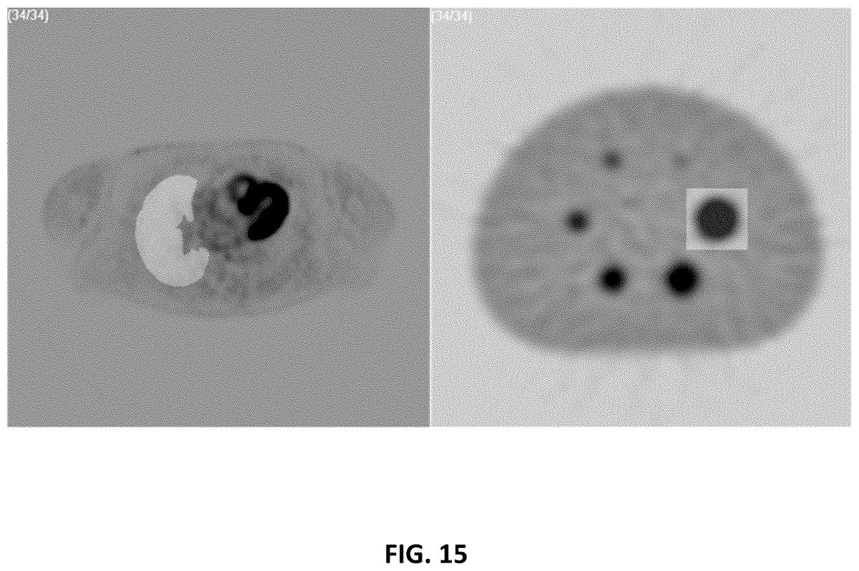

FIG. 15 illustrates the process of transferring the segmented mask to the PET image for a normal Right Lung as an example organ and the phantom used for evaluating TLB.

FIG. 15—Left panel: A transverse FDG-PET image through thorax of a normal subject with right lung mask segmented on the CT image overlaid on the PET image. Right panel: A PET image slice through the “lesion” phantom with a local region of interest placed around one lesion as a mask for disease quantification.

b. Pelvic Organ Segmentation for Radiation Therapy Planning of Prostate Cancer and Other Cancers and Disease Quantification in the Pelvis.

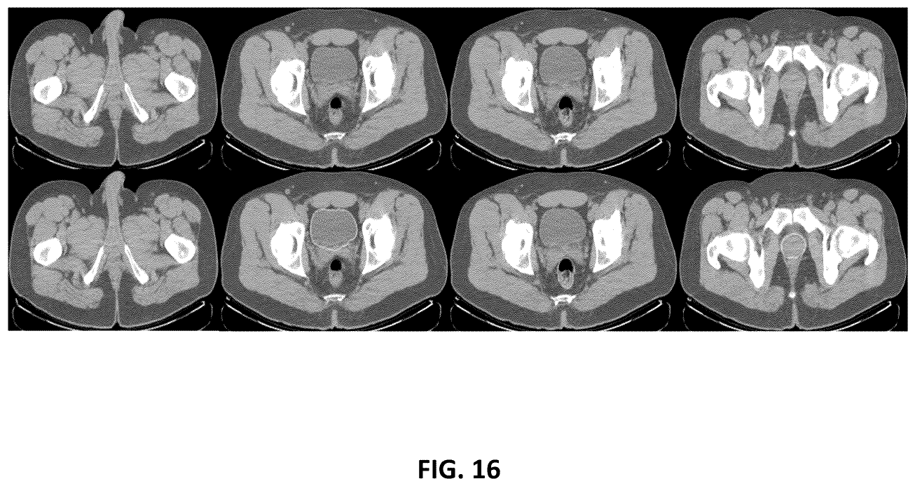

The HI system can be utilized to segment various organs of the pelvis in The HI system can be utilized to segment various organs of the pelvis whether on simulation CT images obtained for radiation therapy planning purposes, or on PET/CT images obtained for diagnostic purposes. For the latter, the segmentation information can then be transferred to the PET images, and subsequently the burden of metastatic cancer in the whole body region and individual organs such as the prostate gland can be quantified. Adaptation of the HI system to this application involved the modifications described in 2a as applied to the organs of the pelvis. Some auto-segmentation results from the modified system are shown in FIG. 16 for Skin, Bladder, Rectum, and Prostate.

FIG. 16 shows examples of auto-segmentation in the pelvis using the HI approach on CT images of FDG-PET/CT acquisitions. A slice from a study (top row) and the segmented organ overlaid on the slice (bottom row). From left to right: Skin, Bladder, Rectum, Prostate.

### 3. Diagnostic C T Imaging

a. Segmentation of the Heart and Epicardial Fat for Studying Pulmonary Hypertension.

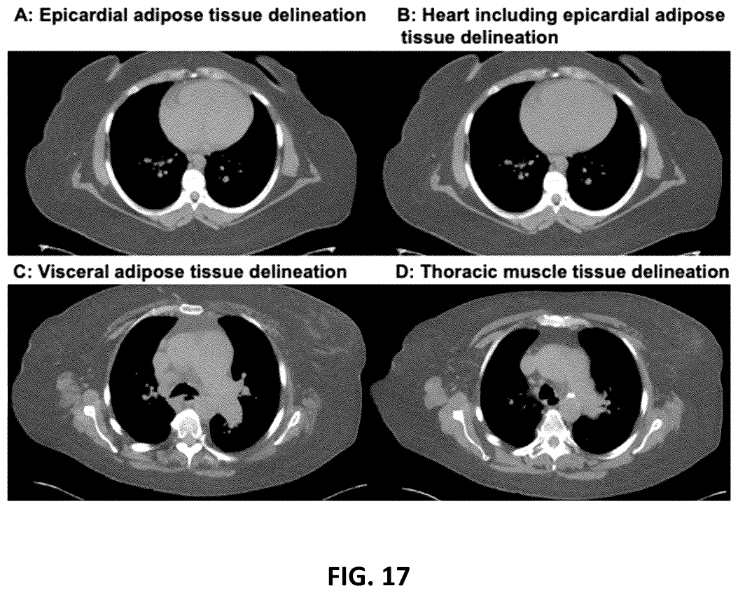

The HI system can be utilized to segment the heart and other cardiovascular structures, epicardial fat, and other components of fat in diagnostic CT images acquired with or without contrast agents. The measurements, particularly subcutaneous and visceral adipose tissues derived from these segmentations, can be used to quantify and study cardiovascular diseases such as pulmonary hypertension on their own or as comorbidities of other conditions such as advanced lung disease and in the setting of lung transplantation. Adaptation of the HI system to this application involved the modifications described in 2a above as applied to the organs and tissue regions of the thorax and to handle diagnostic CT scans. In [8], we demonstrated, based on manual segmentation on one mid-thoracic slice, that lower thoracic visceral adipose tissue volume was associated with a higher risk of pulmonary hypertension in patients with advanced lung disease undergoing evaluation for lung transplantation. With this adaptation of the HI system, such studies can be performed on the whole 3D chest CT scan automatically and for routine clinical use. In FIG. 17, we present an example of 3D segmentation of the heart and different tissue components on a patient chest CT scan.

These are some representative examples of the HI system beyond the specific use case of simulation CT images obtained in the neck or thorax for radiation therapy planning purposes in adults with cancer. In particular, this methodology can be utilized on other imaging modalities (whether low-dose CT, CT from PET/CT, CT from SPECT/CT, diagnostic CT, diagnostic MRI, dynamic MRI, MRI from PET/MRI), can be used in adults and children, and can be applied to a wide variety of other clinical or research applications that require image segmentation for purposes of disease detection, diagnosis, quantification, staging, response assessment, restaging, and outcome prediction.

FIG. 17 shows an example of auto-segmentation of a diagnostic chest CT scan using the HI approach showing the segmentation of epicardial adipose tissue in panel (A), heart including epicardial adipose tissue in panel (B), visceral adipose tissue in panel (C), and thoracic muscle tissue in panel (D).

### REFERENCES

1. Tong Y, Udupa J K, Wileyto P E, McDonough J M, Torigian D A, Campbell R M. Quantitative dynamic Lung M R I (QdMRI) volumetric study of pediatric patients with thoracic insufficiency syndrome, Radiology, 293 (7): 206-213, 2019.
2. Udupa J K, Tong Y, Capraro A, McDonough J M, Mayer O H, Ho S, Wileyto P E, Torigian D A, Campbell R M. Understanding respiratory restrictions as a function of the scoliotic spinal curve in thoracic insufficiency syndrome: A 4D dynamic MR imaging study, Journal of Pediatric Orthopedics, 40 (4): 183-189, 2020.
3. Tong Y, Udupa, JK, McDonough J M, Wu C, Sun C, Xie L, Lott C, Mayer O H, Anari J B, Torigian D A, Cahill P J. Assessment of regional functional effects of surgical treatment in Thoracic Insufficiency Syndrome via dynamic magnetic resonance imaging, Journal of Bone & Joint Surgery, 105: 53-62, 2023.
4. Akhtar Y, Udupa J K, Tong Y, Liu T, Wu C, Odhner D, Mcdonough J M, Lott C, Clark A, Anari J B, Cahill P, Torigian D A. Auto-segmentation of thoraco-abdominal organs in free-breathing pediatric dynamic MRI, Proc, SPIE 12466, Medical Imaging 2023: Image-Guided Procedures, Robotic Interventions, and Modeling, 124660T (3 Apr. 2023): 124660T-1-124660T-7.
5. Hao Y, Udupa J K, Tong Y, Wu C, McDonough J M, Lott C, Anari J B, Cahill P J, Drew A. Torigian D A. Regional diaphragm motion analysis via dynamic MRI, Proc. SPIE 12031, Medical Imaging 2022: Physics of Medical Imaging, 120313F: 12031F-1—12031F-7, 2022.
6. Li J, Udupa J K, Tong Y, Torigian D A. Estimating normal metabolic activity for disease quantification via PET/CT images, Proc. SPIE 12468, Medical Imaging 2023: Biomedical Applications in Molecular, Structural, and Functional Imaging, 124680J (10 Apr. 2023): 124680J-1-124680J-6.
7. Doot R and Kinahan P. SNM lesion phantom report. Technical report, University of Washington, 2007.
8. Al-Naamani N, Pan H M, Anderson M R, Torigian D A, Tong Y, Oyster M, Porteous M K, Palmer S, Arcasoy S M, Diamond J M, Udupa J K, Christie J D, Lederer D J, Kawut S M. Thoracic visceral adipose tissue area and pulmonary hypertension in lung transplant candidates: The Lung Transplant Body Composition Study. Ann Am Thorac Soc. 17 (11): 1393-1400, 2020.

### Additional Examples Section C

### Optimal Strategies for Modeling Anatomy in a Hybrid Intelligence Framework for Auto-Segmentation of Organs

Organ segmentation is a fundamental requirement in medical image analysis. Many methods have been proposed over the past 6 decades for segmentation. A unique feature of medical images is the anatomical information hidden within the image itself. To bring natural intelligence (NI) in the form of anatomical information accumulated over centuries into deep learning (DL) AI methods effectively, we have recently introduced the idea of hybrid intelligence (HI) that combines NI and AI and a system based on HI to perform medical image segmentation. This HI system has shown remarkable robustness to image artifacts, pathology, deformations, etc. in segmenting organs in the Thorax body region in a multicenter clinical study. The HI system utilizes an anatomy modeling strategy to encode NI and to identify a rough container region in the shape of each object via a non-DL-based approach so that DL training and execution are applied only to the fuzzy container region. In this paper, we introduce several advances related to modeling of the NI component so that it becomes substantially more efficient computationally, and at the same time, is well integrated with the DL portion (AI component) of the system. We demonstrate a 9-40 fold computational improvement in the auto-segmentation task for radiation therapy (RT) planning via clinical studies obtained from 4 different RT centers, while retaining state-of-the-art accuracy of the previous system in segmenting 11 objects in the Thorax body region.

### 1. Introduction

Organ segmentation is a fundamental requirement in medical image analysis and the basis for subsequent analysis such as disease quantification and staging, treatment planning, etc. Many methods have been proposed over the past 6 decades for segmentation. Before the deep learning (DL) era, a variety of purely image-based frameworks were proposed [1-5]. To bring in prior knowledge to the segmentation task for overcoming image deficiencies, model-based strategies have been devised [6-10]. In the DL era, Fully Connected Networks (FCNs) first introduced convolutional neural network (CNN)-based methods to the segmentation task [11]. Since then, CNN methods have been continuously developed, mainly focused on network architecture design, loss functions, etc. For medical image segmentation, U-Net [12] is the most popular network architecture, based on which many methods have been proposed. Most of them focus on architecture design, loss function design, attention mechanism, prior knowledge, etc.

A unique feature of medical images is the anatomical information hidden within the image itself for every medical image, which is the biggest difference from natural images. Most methods have ignored such information or have not utilized them fully. To bring natural intelligence (NI) in the form of anatomical information accumulated over centuries into DL (artificial intelligence, AI) methods effectively, we have recently introduced the idea of hybrid intelligence (HI) that combines NI and AI and a system based on HI to perform medical image segmentation [13,14]. This HI system has shown remarkable robustness to image artifacts, pathology, deformations, etc. and generalizability in segmenting organs in the Neck and Thorax body regions in a multicenter clinical evaluation study [13]. The HI system utilized a fuzzy anatomy modeling strategy [10] to encode NI and to identify a rough (fuzzy) container region in the shape of each object via a non-DL-based approach so that DL training and execution are applied only to the container region. In this paper, we introduce several advances related to modeling of the NI component so that it becomes substantially more efficient computationally for object recognition, and at the same time, it can keep object delineation accurate after it is well integrated with the DL portion (AI component) of the system. We demonstrate significant computational improvement in the auto-segmentation task for radiation therapy (RT) planning via clinical studies obtained from 4 different RT centers.

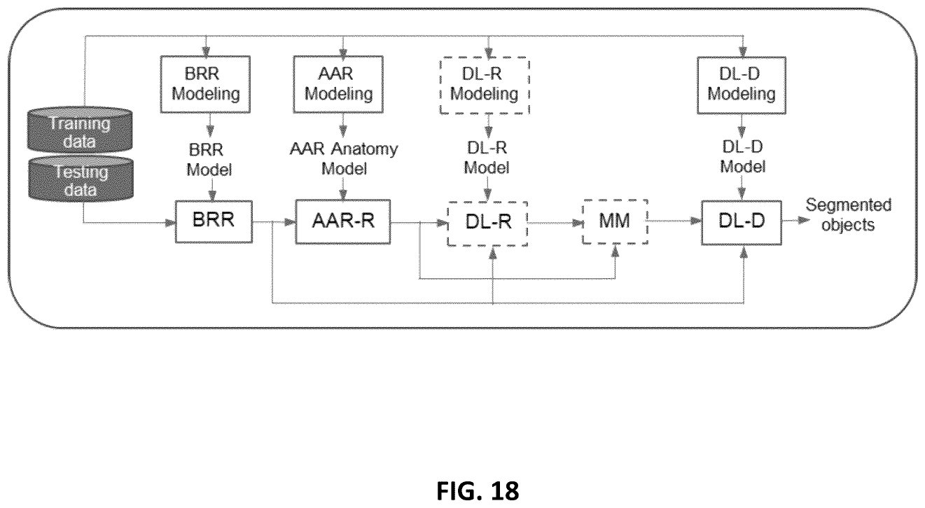

FIG. 18 shows a schematic representation of the proposed HI-system. BRR: Body Region Recognition. AAR-R: AAR object recognition. DL-R: Deep-learning-based object recognition. MM: Model morphing. DL-D: Deep-learning-based object delineation. The dotted modules which were part of the previous system are excluded in the proposed system.

### 2. Materials and Methods

### Methodology Overview

The new HI system is depicted in FIG. 18. The system consists of only the modules shown in solid boxes. The dotted boxes denote modules from the previous system [13] that have been obviated in the new system through the proposed innovations. Prior knowledge (NI) is infused at various modules, majorly in the AAR-R module, which is the main focus of this paper. BRR (Body Region Recognition) is a DL-based strategy which automatically recognizes the body region of interest (Neck, Thorax, etc.), trims the input DICOM image cranio-caudally, and outputs the trimmed image. It utilizes precise definitions of the body region we previously developed. AAR-R (Automatic Anatomy Recognition-Recognition step) performs object model-based (non-DL) recognition (localization) of the object and outputs a container region in the shape of the object. DL-R is a DL-based recognition refinement step which improves on the accuracy of localization by AAR-R and outputs 2D bounding boxes (BBs) tightly enclosing the object. MM is a non-DL-based model morphing step which morphs the object model (container) output by AAR to fit the 2D BBs. DL-D is a DL-based delineation step which uses the output of MM as a container region to which delineation is confined. In the proposed system, DL-R and MM are eliminated and the container output by AAR-R is directly used by DL-D to perform the final segmentation. Equally importantly, the AAR-R module is improved through several novel modeling and recognition strategies so that the total execution time per body region reduced from −17 minutes to 113 seconds for the Thorax body region with 11 organs without sacrificing accuracy.

### 2.1 Materials

This prospective multicenter study was conducted following approval from the Institutional Review Board at the Hospital of the University of Pennsylvania along with a Health Insurance Portability and Accountability Act waiver. 125 thoracic computed tomography (CT) data sets of adult cancer patients undergoing routine radiation therapy planning at our institution (Penn) were utilized for AAR model building and training the DL-D module. Completely independent test studies were gathered from 4 medical centers with roughly equal number of cases from each institution with 104 cases in total for Thorax. The test cases came from 8 different brands and models of scanners, with variable image resolution: pixel size of 0.98 mm to 1.6 mm and slice spacing of 1.5 mm to 4 mm. For the Thorax body region, we considered 11 objects: T-Skn (Thoracic Skin Outer Boundary), ePBT (Extended Proximal Bronchial Tree), RCW (Right Chest Wall), LCW (Left Chest Wall), T-SCn (Thoracic Spinal Canal), LLg (Left Lung), RLg (Right Lung), T-Ao (Thoracic Aorta), Hrt (Heart), T-Es (Thoracic Esophagus), and CPA (Central Pulmonary Arteries). Ground truth segmentations for all cases for all objects were created by several students, trainees (including medical) technicians, and software engineers following strict guidelines based on precise definitions of all objects [13]. This definition step is very crucial for the NI component.

### 2.2 Fuzzy Anatomy Model

The Fuzzy Anatomy Model created in the AAR approach [10] for a body region B is a quintuple, FAM(B)=(H, M, ρ, λ, η), where H denotes a hierarchical arrangement of the objects in B. This arrangement is key to capturing and encoding the geographic layout of the objects. M is a set of fuzzy models, with one fuzzy model FM(O) for each object O in B, where FM(O) represents a fuzzy mask indicating voxel-wise fuzziness of O over the population of samples of O used for model building. ρ represents the parent-to-child geographic relationship of the objects in the hierarchy estimated over the population. λ is a set of scale (size) ranges, one for each object, again estimated over the population, indicating the variation of the size of each object. η includes a host of parameters representing object appearance properties such as the range of variation of image intensity and texture properties, etc., of each object. To recognize objects in a given image I, first the root object (typically skin) is localized in I, then each object (its FM(O)) is positioned in I based solely on the known relationship ρ and its range (this localization method is referred to as one-shot approach). The pose of FM(O) is then refined by an optimal thresholding strategy to fit the model to I [10]. The modified fuzzy model is the output of AAR-R. In this paper, there are 3 key sub-modules to the above established approach in the modeling and recognition steps: 1) Modeling Methods, 2) Simple Hierarchy, and 3) One-shot Recognition.

### 2.3 Alternative Modeling Methods

Central sample (Cs) modeling vs. Fuzzy membership modeling (Fm). In Fm modeling [10], FM(O) is created by scaling each training sample of O to the mean size, translating each sample to the mean centroid location, averaging the binary samples, and then transforming the averaged values to a bona fide fuzzy membership value via a sigmoid mapping. One issue with this approach is that outlier samples unduly influence the resulting FM(O). To overcome this, we propose Cs modeling wherein, among all training samples of O, we select the most “central” sample as the model. We first rescale and translate all samples of O as in Fm modeling. Subsequently, centrality of a sample of O is defined quantitatively by the sum-total of the distance of the sample from all other samples. The sample which yields the smallest distance is taken to be the most central sample. Several choices are available for “distance,” for example, the volume of the exclusive OR region.

### 2.4 Simple Hierarchy

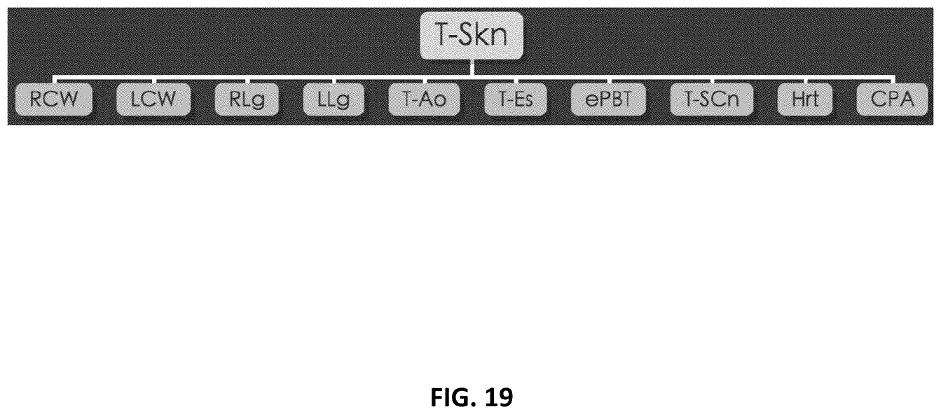

Simple hierarchy (Sh). The hierarchy H in the above quintuple plays a vital role in the recognition step in the AAR approach [10]. We have previously proposed optimal hierarchy (Oh), that is the hierarchy that will yield the smallest total recognition error [15]. Oh mattered in the AAR approach previously when non-DL-based delineation engines were used. However, Oh is computationally more expensive at model building and at recognition. If Sh yields “good enough” recognition results to produce container regions for the DL-D module, then Oh is not necessary. The idea of the simple hierarchy is that all objects are arranged as children of the root object. FIG. 19 illustrates Sh for Thorax.

FIG. 19 shows the simple object hierarchy of the thorax body region.

### 2.5 One-Shot Recognition

One-shot (Os) recognition. The Os recognition strategy is extremely fast compared to Optimal thresholding (Ot) strategy 10] since it makes its decision based on prior knowledge of anatomy, although adjustment to scale based on the parent object already found in I is affected. More importantly, Os allows a parallel implementation where all objects can be recognized simultaneously unlike in Oh where parallelization is hard to implement and not possible to as high degree as in Os.

There is an additional concept at the object level that influences the formulated strategies. We divide objects into two groups based on their form: sparse and non-sparse [10, 13, 14, 15]. Sparse objects are spatially sparse and slender and non-sparse objects are large, space-filling, and compact in form. Sparse objects are more challenging for recognition and delineation than non-sparse objects. In our design, we use the modeling strategies Cs or Fm based on whichever best suits each object—generally Cs for sparse objects and Fm for non-sparse objects. Sparse/non-sparse division constitutes an NI component where knowledge is garnered via anatomy, challenges encountered, and our experience in designing segmentation methods, particularly in the HI framework. By combining the above 3 ideas and with this assignment of AAR modeling strategy for sparse/non-sparse objects, we created one final strategy, which we refer to mnemonically by Sh-Os.

We evaluate the performance of the new HI framework with two metrics: 1) Location error (LE) for object recognition for the AAR-R module, where LE is the distance between the true geometric centroid and the centroid of the recognized object. 2) Dice Similarity Coefficient (DSC) of the final segmentation output of the DL-D module. 3) The time per study (TPS) for segmenting all objects in the body region of interest.

### 3. Results

The simplest method turns out to be Sh-Os in terms of computational efficiency, parallelizability, model complexity, and ease of implementation. In Table 16, we summarize the location errors of the recognition step of 3 different modeling strategies: 1) purely Fuzzy Modeling, 2) purely Central Sampling, and 3) hybrid method using the better one suited for each object. From the results, we can see that in terms of the recognition error, our hybrid method achieved the best overall results.

TABLE 16
Location Error (LE) in mm of different modeling strategies of
objects in Thorax region. (Lower mean LE is better).

| Objects | Fuzzy Modeling | Central Sampling | Hybrid |
|---|---|---|---|
| CPA | 18.544 ± 9.481 | 18.57 ± 8.239 | 18.806 ± 9.264 |
| Hrt | 16.526 ± 12.787 | 16.521 ± 11.497 | 16.526 ± 12.787 |
| LCW | 20.661 ± 7.197 | 18.454 ± 6.935 | 18.716 ± 7.391 |
| LLg | 12.846 ± 8.287 | 12.343 ± 8.062 | 12.846 ± 8.287 |
| RCW | 22.026 ± 6.313 | 18.905 ± 6.882 | 19.084 ± 7.081 |
| RLg | 15.99 ± 9.793 | 13.169 ± 9.393 | 14.452 ± 10.42 |
| T-Ao | 16.884 ± 7.628 | 13.273 ± 7.59 | 13.34 ± 7.665 |
| T-Es | 30.418 ± 10.289 | 45.779 ± 18.115 | 30.418 ± 10.289 |
| T-SCn | 25.386 ± 14.611 | 23.075 ± 14.258 | 22.942 ± 13.86 |
| T-Skn | 4.978 ± 3.624 | 4.978 ± 3.624 | 4.978 ± 3.624 |
| ePBT | 16.148 ± 10.5 | 17.561 ± 9.385 | 16.148 ± 10.5 |
| Overall | 18.213 ± 11.441 | 18.388 ± 14.034 | 17.106 ± 11.322 |

In Table 17, we summarize the key DSC results for objects in the Thorax body region. Since final delineation accuracy is the final arbiter of overall accuracy, we list DSC for each object for the new HI system using the Sh-Os strategy for AAR-R and DSC for the full HI system as depicted in FIG. 18 wherein AAR-R utilized the Oh-Ot strategy. We list the time cost TPS for each system as well. From the table, it is clear that the new HI system shows a level of accuracy that is indistinguishable from the previous HI system [13] while providing significant efficiency advantages. Although we demonstrated efficiency in execution only, it exists to the same degree in training as well (see FIG. 18). The previous HI system was shown (Tables 10-12 in [13]) to have state-of-the-art performance for the Thorax body region. Both systems considered some objects which are extremely challenging to segment which no published works have considered, such as CPA, LCW, RCW, T-Es, and data sets with a wide variety of pathology and artifacts (Table 8 and FIGS. 5 & 6 in [13]). More importantly, we would like to emphasize that in both systems, model building and training were accomplished based on data sets from only one institution. All testing data sets came from other institutions from many different brands and models of scanners. Thus, the HI systems seem to be highly generalizable. We attribute this strength mainly to the hybrid intelligence strategy.

TABLE 17
Dice Similarity Coefficient (DSC) for objects in Thorax body region
for the proposed new HI system and the previous full HI
system [13]. Mean & standard deviation values are listed.
(Higher mean DSC is better.) The last row lists TPS in seconds.

| Objects | New HI system | Previous HI system |
|---|---|---|
| CPA | 0.789, 0.086 | 0.79, 0.10 |
| Hrt | 0.895, 0.075 | 0.92, 0.06 |
| LCW | 0.83, 0.059 | 0.83, 0.06 |
| LLg | 0.951, 0.098 | 0.96, 0.06 |
| RCW | 0.822, 0.059 | 0.82, 0.06 |
| RLg | 0.967, 0.016 | 0.97, 0.02 |
| T-Ao | 0.906, 0.043 | 0.90, 0.04 |
| T-Es | 0.791, 0.09 | 0.79, 0.09 |
| T-SCn | 0.9, 0.048 | 0.89, 0.04 |
| T-Skn | 0.969, 0.025 | — |
| ePBT | 0.872, 0.047 | 0.87, 0.06 |
| Overall (excluding T-Skn) | 0.872, 0.089 | 0.87, 0.09 |
| TPS (seconds) | 25 | 1,006 |

### 4. CONCLUSION

Natural intelligence (NI) with artificial intelligence (AI) (i.e., hybrid intelligence (HI)) has numerous advantages in image segmentation. Objects that are extremely challenging to segment due to poor definition, artifacts, pathology, etc., can be handled robustly due to NI provided explicitly in the form of anatomic guidance. NI also facilitates simplifying computations greatly, thus improving training and execution efficiency. More importantly, as demonstrated in this paper, once we figure out how much location error the DL-D module can tolerate for each object, then considerable savings per study can be achieved without compromising delineation accuracy via the choice of appropriate rough recognition strategies. The integration leads to improved generalizability, which may perhaps obviate the need to resort to federated learning strategies.

### REFERENCES

[1] Beucher, S., “Use of watersheds in contour detection,” In Proceedings of the International Workshop on Image Processing. CCETT (1979).
[2] Kass M, Witkin A, Terzopoulos D. Snakes: Active Contour Models. International Journal of Computer Vision. 1987; 1:321-331.
[3] Mumford D, Shah J. Optimal Approximations by Piecewise Smooth Functions and Associated Variational Problems. Communications of Pure and Applied Mathematics. 1989; 42:577-685.
[4] Malladi R, Sethian J, Vemuri B. Shape Modeling with Front Propagation: A Level Set Approach, IEEE Transactions on Pattern Analysis and Machine Intelligence, 1995; 17:158-175.
[5] Udupa J K, Samarasekera S. Fuzzy Connectedness and Object Definition: Theory, Algorithms, and Applications in Image Segmentation, Graphical Models and Image Processing. 1996; 58:246-261.
[6] Staib L H, Duncan J S. Boundary Finding with Parametrically Deformable Models. IEEE Transactions on Pattern Analysis and Machine Intelligence. 1992; 14:1061-1075.
[7] Gee J, Reivich M, Bajcsy R. Elastically Deforming 3D Atlas to Match Anatomical Brain Images. Journal of Computer Assisted Tomography. 1993; 17:225-236.
[8] Cootes T, Taylor C, Cooper D. Active Shape Models: Their Training and Application, Computer Vision and Image Understanding. 1995; 61:38-59.
[9] Christensen G. Rabbitt R. Miller M. 3-D Brain Mapping Using a Deformable Neuroanatomy. Physics in Medicine and Biology. 1994; 39:609-618.
[10] Udupa J I K, Odhner D, Zao L, et aI. Body-wide Hierarchical Fuzzy Modeling, Recognition, and Delineation of Anatomy in Medical Images. Medical Irnage Analysis. 2014; 18: 752-771.
[11] Long J., Shelhamer B. and Darrell T., “Fully convolutional networks for semantic segmentation,” In Proceedings of the IEEE conference on computer vision and pattern recognition., pp. 3431-3440 (2015).
[12] Ronneberger, O., F ischer, P. and Brox, T., “U-net: Convolutional networks for biomedical image segmentation,” In International Conference on Medical image computing and computer-assisted intervention (pp. 234-241). Springer, Cham (2015),
[13] Udupa J K, Liu T, Jin C, Zhao L, Odhner D, Tong Y, Agrawal V, Pednekar (V, Nag S, Kotia T, Goodman M, Wileyto E P, Mihailidis D, Lukens J N, Berman A T, Stambaugh J, Lim T, Chowdary R, Jalluri D, JabbOur S K, Kim S, Reyhan M, Robinson C, Thorstad W, Choi J I, Press R, Simone C B 2nd, Camaratta J. Owens S. Torigian D A. Combining natural and artificial intelligence for robust automatic anatomy segmentation: Application in neck and thorax auto-contouring for radiation therapy planning, Medical Physics. https://doi:10.1002/mp.15854.
[14] Jin C Udupa J K, Zhao L, Tong Y, Odhner D, Pednekar G V. Nag S, Lewis S. Poole N, Mannikeri S, Covindasamy S, Singh A, Camaratta J, Owens S, Torigian D A. Object recognition in medical irnages via anatomy-guided deep learning, Medical Image Analysis.
[15] Wu X, Udupa J K, Tong Y, Odhner D, Pednekar G V, Simone I I C B, McLaughlin D, Apinorasethkul C, Lukens J, Mihailidis D, Shammo C, James P, Carmaratta J, Torigian D A. AAR-RT—A system for auto-contouring organs at risk on CT images for radiation therapy planning: Principles, design, and large-scale evaluation on head-and-neck and thoracic cancer cases, Medical Image Analysis, 54: 45-62, 2019. https://doi.org/10.1016/j.media.2019.01.008.

The present disclosure may comprise any combination of the following aspects.

Aspect 1. A method comprising or consisting of any combination of the following: receiving imaging data indicative of an object of interest; determining a portion of the imaging data comprising a target body region of the object; determining, based on automatic anatomic recognition and the portion of the imaging data, data indicating one or more objects in the target body region; determining, based on the data indicating the one or more objects and for each of the one or more objects, data indicating a bounding area of an object of one or more objects; modifying, based on data indicating the bounding areas, the data indicating one or more objects in the target body region; determining, based on the modified data indicating one or more objects in the target body region, data indicating a delineation of each of the one or more objects; and causing output of the data indicating the delineation of each of the one or more objects.

Aspect 2. The method of Aspect 1, wherein determining the portion of the imaging data comprising the target body region of the object is based on a first machine learning model trained to trim imaging data to an axial superior boundary and an axial inferior boundary of an indicated target body region.

Aspect 3. The method of any one of Aspects 1-2, wherein data indicating one or more objects in the target body region comprises a fuzzy object model mask indicating recognition an object of the one or more objects.

Aspect 4. The method of any one of Aspects 1-3, wherein determining the data indicating one or more objects in the target body region comprises following a hierarchical objection recognition process based on a fuzzy object model for the target body region, wherein the fuzzy object model indicates a hierarchical arrangement of objects in the target body region.

Aspect 5. The method of any one of Aspects 1-4, wherein automatic anatomic recognition uses a model determined based on human input without the use of a machine learning model trained for anatomic recognition.

Aspect 6. The method of any one of Aspects 1-5, wherein the data indicating a boundary area of an object comprises a set of stacks of two-dimensional boundary boxes having at least one boundary box for each slice of image data comprising the object.

Aspect 7. The method of any one of Aspects 1-6, wherein determining the data indicating the bounding area of an object of the one or more objects comprises inputting to a second machine learning model the portion of the imaging data comprising the target body region of the object and the data indicating one or more objects in the target body region.

Aspect 8. The method of Aspect 7, wherein the second machine learning model comprises a plurality of neural networks each trained for a different area of the object.

Aspect 9. The method of any one of Aspects 1-8, wherein modifying the data indicating one or more objects in the target body region comprises modifying a fuzzy object model mask representing at least one of the one or more objects based on a comparison of the fuzzy object model mask to a bounding area.

Aspect 10. The method of any one of Aspects 1-9, wherein modifying the data indicating one or more objects in the target body region comprises fitting a curve to geometric centers of a plurality of bounding areas from a plurality of image slices and adjusting a fuzzy object model mask based on the curve.

Aspect 11. The method of any one of Aspects 1-10, wherein the data indicating the delineation of each of the one or more objects comprises indications of locations within the imaging data of boundaries of each of the one or more objects within one or more slices of the imaging data.

Aspect 12. The method of any one of Aspects 1-11, wherein the determining the data indicating the delineation of each of the one or more objects comprising inputting the modified data indicating one or more objects in the target body region to a third machine learning model trained to delineate objects.

Aspect 13. The method of any one of Aspects 1-12, wherein causing output of the data indicating a delineation of each of the one or more objects comprises one or more of sending the data indicating the delineation of each of the one or more objects via a network, causing display of the data indicating the delineation of each of the one or more objects, causing the data indicating the delineation of each of the one or more objects to be input to an application, or causing storage of the data indicating the delineation of each of the one or more objects.

Aspect 14. A device comprising: one or more processors; and a memory storing instructions that, when executed by the one or more processors, cause the device to perform the methods of any one of Aspects 1-13.

Aspect 15. A non-transitory computer-readable medium storing instructions that, when executed by one or more processors, cause a device to perform the methods of any one of Aspects 1-13.

Aspect 16. A system comprising: an imaging device configured to generate imaging data of an object of interest; and a computing device comprising one or more processors, and a memory, wherein the memory stores instructions that, when executed by the one or more processors, cause the computing device to perform the methods of any one of claims 1-13.

It is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

The term “or” when used with “one or more of” is used in its inclusive sense (and not in its exclusive sense) so that when used, for example, to connect a list of elements, the term “or” means one, some or all of the elements in the list. The term “or” when used with “at least one of” is used in its inclusive sense (and not in its exclusive sense) so that when used, for example, to connect a list of elements, the term “or” means one, some or all of the elements in the list. For example, the phrases “one or more of A, B, or C” includes any of the following: A, B, C, A and B, A and C, B and C, and A and B and C. Similarly the phrase “one or more of A, B, and C” includes any of the following: A, B, C, A and B, A and C, B and C, and A and B and C. The phrase “at least one of A, B, or C” includes any of following: A, B, C, A and B, A and C, B and C, and A and B and C. Similarly, the phrase “at least one of A, B, and C” includes any of following: A, B, C, A and B, A and C, B and C, and A and B and C.

Components are described that may be used to perform the described methods and systems. When combinations, subsets, interactions, groups, etc., of these components are described, it is understood that while specific references to each of the various individual and collective combinations and permutations of these may not be explicitly described, each is specifically contemplated and described herein, for all methods and systems. This applies to all aspects of this application including, but not limited to, operations in described methods. Thus, if there are a variety of additional operations that may be performed it is understood that each of these additional operations may be performed with any specific embodiment or combination of embodiments of the described methods.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described herein with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded on a general-purpose computer, special-purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

The various features and processes described above may be used independently of one another, or may be combined in various ways. All possible combinations and sub-combinations are intended to fall within the scope of this disclosure. In addition, certain methods or process blocks may be omitted in some implementations. The methods and processes described herein are also not limited to any particular sequence, and the blocks or states relating thereto may be performed in other sequences that are appropriate. For example, described blocks or states may be performed in an order other than that specifically described, or multiple blocks or states may be combined in a single block or state. The example blocks or states may be performed in serial, in parallel, or in some other manner. Blocks or states may be added to or removed from the described example embodiments. The example systems and components described herein may be configured differently than described. For example, elements may be added to, removed from, or rearranged compared to the described example embodiments.

It will also be appreciated that various items are illustrated as being stored in memory or on storage while being used, and that these items or portions thereof may be transferred between memory and other storage devices for purposes of memory management and data integrity. Alternatively, in other embodiments, some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), etc. Some or all of the modules, systems, and data structures may also be stored (e.g., as software instructions or structured data) on a computer-readable medium, such as a hard disk, a memory, a network, or a portable media article to be read by an appropriate device or via an appropriate connection. The systems, modules, and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

While the methods and systems have been described in connection with preferred embodiments and specific examples, it is not intended that the scope be limited to the particular embodiments set forth, as the embodiments herein are intended in all respects to be illustrative rather than restrictive.

It will be apparent to those skilled in the art that various modifications and variations may be made without departing from the scope or spirit of the present disclosure. Other embodiments will be apparent to those skilled in the art from consideration of the specification and practices described herein. It is intended that the specification and example figures be considered as exemplary only, with a true scope and spirit being indicated by the following claims.

What is claimed:

1. A method comprising:
receiving imaging data indicative of an object of interest;
determining a portion of the imaging data comprising a target body region of the object;
determining, based on automatic anatomic recognition and the portion of the imaging data, data indicating one or more objects in the target body region;
determining, based on the data indicating the one or more objects and for each of the one or more objects, data indicating a bounding area of an object of one or more objects;
modifying, based on data indicating the bounding areas, the data indicating one or more objects in the target body region;
determining, based on the modified data indicating one or more objects in the target body region, data indicating a delineation of each of the one or more objects; and
causing output of the data indicating the delineation of each of the one or more objects.

2. The method of claim 1, wherein determining the portion of the imaging data comprising the target body region of the object is based on a first machine learning model trained to trim imaging data to an axial superior boundary and an axial inferior boundary of an indicated target body region.

3. The method of claim 1, wherein data indicating one or more objects in the target body region comprises a fuzzy object model mask indicating recognition an object of the one or more objects.

4. The method of claim 1, wherein determining the data indicating one or more objects in the target body region comprises following a hierarchical objection recognition process based on a fuzzy object model for the target body region, wherein the fuzzy object model indicates a hierarchical arrangement of objects in the target body region.

5. The method of claim 1, wherein automatic anatomic recognition uses a model determined based on human input without the use of a machine learning model trained for anatomic recognition.

6. The method of claim 1, wherein the data indicating a boundary area of an object comprises a set of stacks of two-dimensional boundary boxes having at least one boundary box for each slice of image data comprising the object.

7. The method of claim 1, wherein determining the data indicating the bounding area of an object of the one or more objects comprises inputting to a second machine learning model the portion of the imaging data comprising the target body region of the object and the data indicating one or more objects in the target body region.

8. The method of claim 7, wherein the second machine learning model comprises a plurality of neural networks each trained for a different area of the object.

9. The method of claim 1, wherein modifying the data indicating one or more objects in the target body region comprises modifying a fuzzy object model mask representing at least one of the one or more objects based on a comparison of the fuzzy object model mask to a bounding area.

10. The method of claim 1, wherein modifying the data indicating one or more objects in the target body region comprises fitting a curve to geometric centers of a plurality of bounding areas from a plurality of image slices and adjusting a fuzzy object model mask based on the curve.

11. The method of claim 1, wherein the data indicating the delineation of each of the one or more objects comprises indications of locations within the imaging data of boundaries of each of the one or more objects within one or more slices of the imaging data.

12. The method of claim 1, wherein the determining the data indicating the delineation of each of the one or more objects comprising inputting the modified data indicating one or more objects in the target body region to a third machine learning model trained to delineate objects.

13. The method of claim 1, wherein causing output of the data indicating a delineation of each of the one or more objects comprises one or more of sending the data indicating the delineation of each of the one or more objects via a network, causing display of the data indicating the delineation of each of the one or more objects, causing the data indicating the delineation of each of the one or more objects to be input to an application, or causing storage of the data indicating the delineation of each of the one or more objects.

14. A device comprising:
one or more processors; and
a memory storing instructions that, when executed by the one or more processors, cause the device to:
receive imaging data indicative of an object of interest;
determine a portion of the imaging data comprising a target body region of the object;
determine, based on automatic anatomic recognition and the portion of the imaging data, data indicating one or more objects in the target body region;
determine, based on the data indicating the one or more objects and for each of the one or more objects, data indicating a bounding area of an object of one or more objects;
modify, based on data indicating the bounding areas, the data indicating one or more objects in the target body region;
determine, based on the modified data indicating one or more objects in the target body region, data indicating a delineation of each of the one or more objects; and
cause output of the data indicating the delineation of each of the one or more objects.

15. The device of claim 14, wherein the instructions that, when executed by the one or more processors, cause the device to determine the portion of the imaging data comprising the target body region of the object comprises instructions that, when executed by the one or more processors, cause the device to determine the portion of the imaging data comprising the target body region of the object based on a first machine learning model trained to trim imaging data to an axial superior boundary and an axial inferior boundary of an indicated target body region.

16. The device of claim 14, wherein automatic anatomic recognition uses a model determined based on human input without the use of a machine learning model trained for anatomic recognition.

17. The device of claim 14, wherein the instructions that, when executed by the one or more processors, cause the device to determine the data indicating the bounding area of an object of the one or more objects comprises instructions that, when executed by the one or more processors, cause the device to input to a second machine learning model the portion of the imaging data comprising the target body region of the object and the data indicating one or more objects in the target body region.

18. A system comprising:
an imaging device configured to generate imaging data of an object of interest; and
a computing device comprising one or more processors, and a memory, wherein the memory stores instructions that, when executed by the one or more processors, cause the computing device to:
receive the imaging data indicative of the object of interest;
determine a portion of the imaging data comprising a target body region of the object;
determine, based on automatic anatomic recognition and the portion of the imaging data, data indicating one or more objects in the target body region;
determine, based on the data indicating the one or more objects and for each of the one or more objects, data indicating a bounding area of an object of one or more objects;
modify, based on data indicating the bounding areas, the data indicating one or more objects in the target body region;
determine, based on the modified data indicating one or more objects in the target body region, data indicating a delineation of each of the one or more objects; and
cause output of the data indicating the delineation of each of the one or more objects.

19. The system of claim 18, wherein the computing device is configured to determine the portion of the imaging data comprising the target body region of the object based on a first machine learning model trained to trim imaging data to an axial superior boundary and an axial inferior boundary of an indicated target body region.

20. The system of claim 18, wherein automatic anatomic recognition uses a model determined based on human input without the use of a machine learning model trained for anatomic recognition.
